(19) United States
(12) Patent Application Publication
(10) Pub. No.: US 2021/0308028 A1
(43) Pub. Date: Oct. 7, 2021

(54) Compositions and Methods for Maintaining and Protecting Tissue Integrity and Barrier Function

(71) Applicant: Colgate-Palmolive Company, New York, NY (US)

(72) Inventors: Ying Yang, Monmouth Junction, NJ (US); James Masters, Ringoes, NJ (US); Harsh Mahendra Trivedi, Hillsborough, NJ (US); Tulika Sarma, Hillsborough, NJ (US)

(73) Assignee: Colgate-Palmolive Company, New York, NY (US)

(21) Appl. No.: 17/301,257

(22) Filed: Mar. 30, 2021

Related U.S. Application Data
(60) Provisional application No. 63/001,872, filed on Mar. 30, 2020.
(60) Provisional application No. 63/198,608, filed on Oct. 29, 2020.

(51) Int. Cl. A61K 8/58; A61K 8/44; A61K 8/21; A61K 8/27; A61Q 11/00

(52) U.S. Cl. CPC A61K 8/58; A61K 8/44; A61Q 11/00; A61K 8/27; A61K 8/21

(57) ABSTRACT

Method of improving tissue integrity of tissue that functions as a barrier are provided. Method of protecting and maintaining tissue integrity of tissue that functions as a barrier are provided. Method of repairing damage to tissue that functions as a barrier, and to restoring and improving function of such damaged tissue are provided. Methods of improving, maintaining, protecting, repairing and/or restoring the integrity of tissue which functions as a barrier and repair of damage to and healing of wounds to such tissue improves, maintains, protects, repairs and restores immunity. The methods comprising contacting the tissue with an effective amount of a composition comprising one or more sources of zinc ions such as zinc oxide, zinc citrate, or combination thereof zinc oxide and zinc citrate, and optionally further comprising arginine and/or one or more neutral amino acids.

### CROSS-REFERENCE TO RELATED APPLICATIONS

This application claims priority to U.S. Provisional Application No. 63/001,872 filed Mar. 30, 2020, which is incorporated herein by reference in its entirety, and U.S. Provisional Application No. 63/198,608 filed Oct. 19, 2020, which is incorporated herein by reference in its entirety.

### BACKGROUND

Tissue integrity is essential for maintaining the effective functioning of such tissue, especially when such tissue functions as a barrier. Epithelial tissue integrity is essential for healthy skin. The integrity of oral soft tissue is important for maintaining the barrier function of such tissue in the oral cavity, as it covers about 80% of the oral cavity surface. The integrity of oral soft tissue and its barrier function are essential to overall health.

The immune system, which includes both innate and adaptive immunity, is a defense system which protects a host from pathogens. Innate immunity is the first line of defense against non-self pathogens. The innate immune system consists of physical, chemical and cellular defenses that function to immediately prevent the spread and movement of foreign pathogens throughout the body. One feature of the innate immune system provides physical barriers to infectious and pathogenic agents via physical measures which function as first-line physical barrier.

The oral cavity harbors a complex microbiome that includes both beneficial microorganisms as well as potentially harmful pathogens. Various sites in the oral cavity are colonized by a wide range of microbial species, mainly bacteria, which interact with each other and with host cells, contributing to physiological and pathological conditions. These microorganisms colonize the hard and soft tissues of the oral cavity in biofilms under an ecological equilibrium with the host. Disruption of this homeostasis leading to dysbiosis has been linked to several oral disorders such as dental caries and periodontal diseases.

Periodontal diseases that affect the tooth-supporting structures are the most common chronic inflammatory disorders in adults. Gingivitis is characterized by an inflammatory condition limited to the unattached gingiva that is reversible upon the improvement of oral hygiene. Gingivitis can develop into periodontitis, a progressive and destructive disease that affects all supporting tissues of the teeth, including the periodontal ligament and alveolar bone. Periodontitis proceeds cyclically with periods of activity and quiescence until therapy is initiated. Accumulating studies point to a relationship between periodontal diseases and systemic health problems, including cardiovascular and respiratory diseases, type 2 diabetes, preterm birth/low birth weight, and colorectal disease. Two etiological components contribute to periodontal disease: a limited group of Gram-negative anaerobic bacteria that initiate the disease, and the uncontrolled and exaggerated inflammatory response of resident and immune cells to the presence of these pathogens and their toxins. This results in the secretion of a large array of inflammatory mediators and matrix metalloproteinases (MMPs) that modulate destruction of the tooth-supporting tissues.

The first line of host defense against both opportunistic and pathogenic microorganisms colonizing the oral cavity is the oral epithelium. The oral epithelium creates a physical protective barrier between the underlying connective tissue and pathogens, and thereby plays an active role in maintaining oral health and oral immunity. Oral soft tissue problems such as gum disease involve bacteria and host cell interactions. The gingival epithelium, a stratified squamous tissue that acts as an interface between the external environment and the underlying connective tissue, plays an active role in the maintenance of oral health, and particularly periodontal health. The gingival epithelium interacts with and protects against invasive periodontal pathogens and their toxic products.

The physical epithelial barrier is composed of closely opposed cells that connect neighboring cells to each other by specialized intercellular tight junctions. The cell junctions function as intercellular pathways that selectively permit the movement of molecules through cellular layers. These tight junctions seal the paracellular space, blocking the pathway to bacteria and toxins while allowing the flux of water and nutrients. The intercellular tight junction, which is composed of specialized transmembrane proteins that regulate transepithelial permeability, is the primary cellular determinant of epithelial barrier integrity and function. In terms of biological function, cell junctions form a barrier between apical and basolateral cell surfaces and are crucial in the development and function of epithelial tissues.

Chemical, mechanical, and biological factors from pathogens may disrupt the oral tissue integrity, leading to an increase in tissue permeability, and subsequently increasing risk of tissue damage and infection. Oral pathogens employ different strategies to compromise the structural and functional integrity of the oral epithelium. The integrity and barrier functions of this epithelium may be compromised through destruction of the specialized intercellular tight junctions by periodontopathogens.

Proteolytic enzymes from pathogenic bacteria are involved in the degradation of cell-to-cell junctions and the disruption of the epithelial barrier. Type I collagen makes up approximately 60% of the tissue volume of periodontal tissues. The collagenolytic activity of some pathogenic bacteria has been attributed to the action of gingipains, which are both secreted and cell-bound. Some proteases such as matrix metallopeptidases (MMPs), also known as matrix metalloproteinases, are among the critical virulent factors to degrade gum tissue in perio-etiology. Pathogenic bacteria-induced tissue damage arises in part from induced protease activity by MMPs.

Pathogens may have hemolytic activity that lyses erythrocytes and releases hemoglobin. Such hemolytic activity is considered a virulence determinant since it provides an iron source to pathogens that promotes their proliferation, particularly in subgingival sites. Moreover, hemoglobin has been reported to synergize with lipopolysaccharides from pathogens to amplify the inflammatory response of human macrophages.

Once the integrity of the oral epithelium is disrupted, pathogens can interact with deeper connective tissues and trigger a marked pro-inflammatory response that modulates tissue destruction and that further compromises barrier integrity and function. Proinflammatory cytokines PGE2, IL-β, IL-6, IL-8, GM-CSF and TNF-α, create an environment that helps disease progression. Three proinflammatory cytokines, IL-β, IL-6, and tumor necrosis factor-α (TNF-α), appear to have a central role in periodontal tissue destruction. Immune and inflammatory responses in response to chronic infection can result in uncontrolled secretion of cytokines, leading to chronic inflammation and periodontal tissue destruction. As a result, most damage seen in periodontitis, for example, is host mediated.

The proinflammatory cytokines contribute to tissue destruction by a number of pathways including regulating immune function, stimulating bone loss, inhibiting bone forming, and regulating homeostasis of periodontal tissues. Proinflammatory cytokines have addition deleterious effects by reduce tissue integrity and barrier function, by decreasing collagen synthesis, upregulating expression of various lytic enzymes, such as matrix metalloproteinases and downregulating expression of their inhibitors.

Disruption of the integrity of the oral epithelium can also allow bacteria and their toxins to enter the bloodstream, migrate to extra-oral sites, and cause systemic complications.

Oral health in general and periodontal health in particular can be promoted by 1) eliminating/neutralizing pathogens such as periodontal pathogens, and 2) improving innate immunity by maintaining, protecting, repairing, improving and/or reinforcing epithelial barrier function. Modulating the epithelial barrier function effectively reduces the risk of infection and helps prevent oral diseases. Inhibition of collagen degradation and inhibition of induction of protease activity of perio-pathogenic bacteria may contribute to reducing the tissue destructive process mediated by oral pathogens and reduce damage to oral tissue. Inhibition of hemolysis may contribute to reducing the levels of pro-inflammatory mediators in periodontal sites in addition to attenuate growth of pathogenic bacteria. Inhibiting or reducing inflammation triggered by pro-inflammatory cytokines reduces tissue destruction caused by inflammation and thus promotes oral health in general and periodontal health in particular. Maintaining, protecting, repairing, improving and/or reinforcing tissue integrity and epithelial barrier function provides improved oral immunity.

In one particular example, oral soft tissue problems such as gum disease involve gram-negative anaerobic bacteria and host cell interactions. Porphyromonas gingivalis, a late colonizer of the periodontal biofilm, has been strongly associated with the chronic form of periodontitis. This Gram-negative bacterium produces a broad array of virulence factors that contribute to tissue colonization and destruction, host defense perturbation, and inflammatory tissue destruction. Epithelial cells and fibroblasts are the predominant cells of periodontal tissues and serve as a first line of defense against periodontopathogens. They act as a mechanical barrier against bacterial invasion in addition to secreting different classes of inflammatory mediators and tissue-destructive enzymes in response to pathogen stimulation.

The pathogenic properties of P. gingivalis include proteolytic and hemolytic activities that cause damage to the epithelial barrier integrity. P. gingivalis has been shown to induce marked damages in an in vitro model of oral keratinocyte barrier. Strong evidence points to the cysteine proteases (gingipains) produced by P. gingivalis as major contributors of the deleterious effect through degradation of tight junction proteins. This periodontopathogen induces breakdown of the gingival keratinocyte barrier resulting in bacterial translocation. Once the cell-cell interactions are disorganized, bacteria can invade deeper oral tissues, triggering an inflammatory response and establishing a chronic infection that may be associated with a migration of pathogens to non-oral sites.

When the immune and inflammatory responses do not stop the progression of the periodontal infection, uncontrolled secretion of cytokines occurs, leading to chronic inflammation and host mediated periodontal tissue destruction and periodontal damage. TNF-α, a multi-role cytokine identified in inflamed periodontal tissue, gingival crevicular fluid, and saliva plays a prominent role in the pathogenic process of periodontitis. TNF-α can modulate tissue and bone destruction by up-regulating the expression of matrix metalloproteinases (MMPs) and receptor activator of nuclear factor kappa-B ligand (RANKL). In vitro evidence suggests that TNF-α may exert deleterious effects on the oral epithelium through amplification of the host inflammatory response and destruction of the keratinocyte barrier integrity.

Given the crucial protective role played by the oral epithelial barrier, compounds endowed with a capacity to enhance or protect tissue barrier function are of great interest as potential oral care products. Conditions or substances with an ability to attenuate this destructive process or to promote the restoration of an intact keratinocyte barrier through cell proliferation and migration may be of high interest for maintaining or recovering an efficient gingival barrier. Compositions and methods that reinforce the oral barrier such as oral epithelial barrier, for example gingival epithelial barrier, are useful to protect it from damage and to promote oral health generally. Compositions and methods that protect oral tissue from tissue damage caused by perio-pathogenic bacteria promote oral health. Compositions and methods that protect oral tissue from proinflammatory cytokine induced tissue damage promote oral health. Compositions and methods that inhibit induction of proteases from perio-pathogenic bacteria promote oral health. Compositions and methods inhibit P. gingivalis collagenase activity, proteolytic enzymes to destroy gum tissue; translocation and hemolytic property promote oral health. Compositions and methods that attenuate this destructive process or promote the restoration of an intact keratinocyte barrier through cell proliferation and migration facilitate maintenance or recovery of an efficient gingival barrier.

### BRIEF SUMMARY

Oral immunity may be maintained, improved, repaired and/or restored by performing methods of improving, maintaining, protecting, repairing and/or restoring tissue integrity of tissue that functions as a barrier and repairing damage and healing wounds to such tissue.

Methods of improving tissue integrity of tissue that functions as a barrier are provided. The methods comprise contacting the tissue with an effective amount of a composition comprising one or more sources of zinc ions, such as for example, one or more sources of zinc ions selected from the group consisting of: zinc oxide and zinc citrate and optionally further comprising arginine and/or one or more of alanine, asparagine, cysteine, glutamine, glycine, isoleucine, leucine, methionine, phenylalanine, proline, serine, threonine, tryptophan, tyrosine, valine and amino acids having an isoelectric point of pH 5.0 to 7.0. The improvement in tissue integrity may render the specialized transmembrane proteins of the intercellular tight junction more effective at regulating transepithelial permeability and/or provide improved paracellular permeability function of the intercellular tight junction.

Method of protecting and maintaining tissue integrity of tissue that functions as a barrier are provided. The methods comprise contacting the tissue with an effective amount of a composition comprising one or more sources of zinc ions, such as for example, one or more sources of zinc ions selected from the group consisting of: zinc oxide and zinc citrate and optionally further comprising arginine and/or one or more of alanine, asparagine, cysteine, glutamine, glycine, isoleucine, leucine, methionine, phenylalanine, proline, serine, threonine, tryptophan, tyrosine, valine and amino acids having an isoelectric point of pH 5.0 to 7.0. The methods of protecting and maintaining tissue integrity of the tissue that functions as a barrier may render the barrier more effective against pathogenic bacteria. The barrier may be rendered more effective at regulating transepithelial permeability in the presence of pathogenic bacteria and/or the barrier may not undergo a reduction in paracellular permeability function of the intercellular tight junction the presence of pathogenic bacteria. The methods of protecting and maintaining tissue integrity of the tissue that functions as a barrier may render more effective against deleterious effects of proinflammatory cytokines such as PGE2, IL-β, IL-6, IL-8, GM-CSF and TNF-α. A barrier more effective against deleterious effects of proinflammatory cytokines may be more effective at regulating transepithelial permeability in the presence of proinflammatory cytokines and/or does not undergo a reduction in paracellular permeability function of the intercellular tight junction the presence of proinflammatory cytokines. The methods of protecting and maintaining tissue integrity of the tissue that functions as a barrier may render the barrier more effective against virulent factors produced by pathogenic bacteria such as proteases. The barrier may be rendered more effective at regulating transepithelial permeability in the presence of such virulent factors and/or the barrier may not undergo a reduction in paracellular permeability function of the intercellular tight junction the presence of such virulent factors. The methods of protecting and maintaining tissue integrity of the tissue that functions as a barrier may include inhibiting factors released by or otherwise associated with pathogenic bacteria such as proteases of pathogenic bacteria and/or inhibiting tissue invasion by pathogenic bacteria that is facilitated by proteases of pathogenic bacteria and/or inhibiting cell lysis activity, such as hemolytic activity by pathogenic bacteria and/or inhibiting induction of protease production, such as MMP production by host cells, and/or host cell protease activity, such as host cell MMP activity in response to the presence of pathogenic bacteria.

Methods of repairing damage to the barrier and restoring its functions are provided. The methods comprise contacting the tissue with an effective amount of a composition comprising one or more sources of zinc ions, such as for example, one or more sources of zinc ions selected from the group consisting of: zinc oxide and zinc citrate and optionally further comprising arginine and/or one or more of alanine, asparagine, cysteine, glutamine, glycine, isoleucine, leucine, methionine, phenylalanine, proline, serine, threonine, tryptophan, tyrosine, valine and amino acids having an isoelectric point of pH 5.0 to 7.0. The methods promote the restoration of the keratinocyte barrier through cell proliferation and migration. The methods support barrier maintenance and promote recovery an efficient gingival barrier damaged by the secretion of a large array of inflammatory mediators, such as TNF-α, and matrix metalloproteinases (MMPs) that modulate destruction of the tooth-supporting tissues. The methods promote healing of the damage that results from deleterious effects of TNF-α on the oral epithelium through amplification of the host inflammatory response and destruction of the keratinocyte barrier integrity. The methods attenuate the TNF-α-induced barrier dysfunction.

### BRIEF DESCRIPTION OF THE DRAWINGS

FIG. 1 shows results from experiments described in Example 1. The data in the graph show the TER ratio of zinc citrate-zinc oxide (Dual Zinc—0.5% Zinc citrate+1.0% Zinc oxide) after SLS exposure over before SLS exposure.

FIG. 2 shows results from experiments described in Example 1. The data in the graph show TER ratio of zinc citrate-zinc oxide (Dual Zinc—0.5% Zinc citrate+1.0% Zinc oxide) 60 hours after SLS exposure over immediately after SLS exposure.

FIG. 3 shows results from experiments described in Example 1. The data in the graph show TER ratio of arginine (1.5%) after SLS exposure over before SLS exposure

FIG. 4 shows results from experiments described in Example 1. The data in the graph show TER ratio of arginine (1.5%) 60 hours after SLS exposure over immediately after SLS exposure

FIG. 5 shows results from experiments described in Example 1. The data in the graph show that tissue treated with zinc citrate-zinc oxide plus arginine (DZA—Dual Zinc+Arginine 0.5% Zinc citrate+1.0% Zinc oxide+1.5% Arginine) toothpaste formulation results in better tissue integrity than tissue treated with regular fluoride toothpaste (CDC).

FIG. 6 shows data from TER assay experiments described in Example 3. The y-axis refers to TER measured as a percent of the initial value measured. The x-axis refers to the three dilutions of zinc oxide formulation tested plus the negative control. TER was measured at multiple time points for each dilution as indicated on bar graph.

FIG. 7 shows data from experiments assessing paracellular permeability to FITC-dextran described in Example 3. The y-axis refers to FD-4 Relative fluorescence units measured. The x-axis refers to time point when measurements were taken over a 48-hour period. The data in the graph is for the three dilutions of zinc oxide formulation tested plus the negative control.

FIG. 8 shows photographs of immunofluorescence of tight junction proteins from experiments described in Example 3. The photos are of either cells treated with one of three dilutions of zinc oxide formulation or the negative control cells. The photos in the top row were stained for Occludin, which is green in original color. The photos in the bottom row were stained for Zonula Occludens-1, which is red in original color.

FIG. 9 shows data from TER assay experiments described in Example 3. The y-axis refers to TER measured as a percent of the initial value measured. The x-axis refers to the three dilutions of zinc citrate formulation tested plus the negative control. TER was measured at multiple time points for each dilution as indicated on bar graph.

FIG. 10 shows data from experiments assessing paracellular permeability to FITC-dextran described in Example 3. The y-axis refers to FD-4 Relative fluorescence units measured. The x-axis refers to time point when measurements were taken over a 48-hour period. The data in the graph is for the three dilutions of zinc citrate formulation tested plus the negative control.

FIG. 11 shows photographs of immunofluorescence of tight junction proteins from experiments described in Example 3. The photos are of either cells treated with one of three dilutions of zinc citrate formulation or the negative control cells. The photos in the top row were stained for Occludin, which is green in original color. The photos in the bottom row were stained for Zonula Occludens-1, which is red in original color.

FIG. 12 shows data from TER assay experiments described in Example 3. The y-axis refers to TER measured as a percent of the initial value measured. The x-axis refers to the percentage of zinc oxide, zinc citrate and arginine formulation in the sample tested plus the negative control. TER was measured at multiple time points for each dilution as indicated on bar graph.

FIG. 13 shows data from experiments assessing paracellular permeability to FITC-dextran described in Example 3. The y-axis refers to FD-4 Relative fluorescence units measured. The x-axis refers to time point when measurements were taken over a 48-hour period. The data in the graph refers to the percentage of zinc oxide, zinc citrate and arginine formulation in the sample tested plus the negative control.

FIG. 14 shows photographs of immunofluorescence of tight junction proteins from experiments described in Example 3. The photos are of either cells treated with samples having 0.0% (negative control), 0.1% and 0.2% of zinc oxide, zinc citrate and arginine formulation. The photos in the top row were stained for Occludin, which is green in original color. The photos in the bottom row were stained for Zonula Occludens-1, which is red in original color.

FIG. 15 shows data from TER assay experiments described in Example 4. The y-axis refers to TER measured as a percent of the initial value measured. The x-axis refers to a first control (no formulation, no P. gingivalis), a second control (no formulation but with P. gingivalis) two controls (no formulation tested) and the three dilutions of zinc oxide formulation in combination with P. gingivalis tested.

FIG. 16 shows data from experiments assessing paracellular permeability to FITC-dextran described in Example 4. The y-axis refers to FD-4 Relative fluorescence units measured. The x-axis refers to time point when measurements were taken over a 48-hour period. The data in the graph is for a first control (no formulation tested/no P. gingivalis), a second control (no formulation tested but with P. gingivalis) and the three dilutions of zinc oxide formulation together with P. gingivalis.

FIG. 17 shows photographs of immunofluorescence of tight junction proteins from experiments described in Example 4. The photos are of either cells treated with a first control (no formulation tested/no P. gingivalis), a second control (no formulation tested but with P. gingivalis) or one of three dilutions of zinc oxide formulation together with P. gingivalis. The photos in the top row were stained for Occludin, which is green in original color. The photos in the bottom row were stained for Zonula Occludens-1, which is red in original color.

FIG. 18 shows data from TER assay experiments described in Example 4. The y-axis refers to TER measured as a percent of the initial value measured. The x-axis refers to a first control (no formulation, no P. gingivalis), a second control (no formulation but with P. gingivalis) two controls (no formulation tested) and the three dilutions of zinc citrate formulation in combination with P. gingivalis tested.

FIG. 19 shows data from experiments assessing paracellular permeability to FITC-dextran described in Example 4. The y-axis refers to FD-4 Relative fluorescence units measured. The x-axis refers to time point when measurements were taken over a 48-hour period. The data in the graph is for a first control (no formulation tested/no P. gingivalis), a second control (no formulation tested but with P. gingivalis) and the three dilutions of zinc citrate formulation together with P. gingivalis.

FIG. 20 shows photographs of immunofluorescence of tight junction proteins from experiments described in Example 4. The photos are of either cells treated with a first control (no formulation tested/no P. gingivalis), a second control (no formulation tested but with P. gingivalis) or one of three dilutions of zinc citrate formulation together with P. gingivalis. The photos in the top row were stained for Occludin, which is green in original color. The photos in the bottom row were stained for Zonula Occludens-1, which is red in original color.

FIG. 21 shows data from TER assay experiments described in Example 4. The y-axis refers to TER measured as a percent of the initial value measured. The x-axis refers to a first control (no formulation, no P. gingivalis), a second control (no formulation but with P. gingivalis) two controls (no formulation tested) and one of four percentages of the zinc oxide-zinc citrate-arginine formulation in combination with P. gingivalis in the sample tested.

FIG. 22 shows data from experiments assessing paracellular permeability to FITC-dextran described in Example 4. The y-axis refers to FD-4 Relative fluorescence units measured. The x-axis refers to time point when measurements were taken over a 48-hour period. The data in the graph is for a first control (no formulation tested/no P. gingivalis), a second control (no formulation tested but with P. gingivalis) and the four percentages of the zinc oxide-zinc citrate-arginine formulation in the sample tested together with P. gingivalis.

FIG. 23 shows photographs of immunofluorescence of tight junction proteins from experiments described in Example 4. The photos are of either cells treated with a first control (no formulation tested/no P. gingivalis), a second control (no formulation tested but with P. gingivalis) or one of four percentages of the zinc oxide-zinc citrate-arginine formulation in the sample tested together with P. gingivalis. The photos in the top row were stained for Occludin, which is green in original color. The photos in the bottom row were stained for Zonula Occludens-1, which is red in original color.

FIG. 24 shows data from TER assay experiments described in Example 5. The y-axis refers to TER measured as a percent of the initial value measured. The x-axis refers to a first control (no formulation, no TNFα), a second control (no formulation but with TNFα) and the three dilutions of zinc oxide formulation in combination with TNFα tested.

FIG. 25 shows data from experiments assessing paracellular permeability to FITC-dextran described in Example 5. The y-axis refers to FD-4 Relative fluorescence units measured. The x-axis refers to time point when measurements were taken over a 48-hour period. The data in the graph is for a first control (no formulation tested/no TNFα), a second control (no formulation tested but with TNFα) and the three dilutions of zinc oxide formulation in combination with TNFα.

FIG. 26 shows photographs of immunofluorescence of tight junction proteins from experiments described in Example 5. The photos are of either cells treated with a first control (no formulation tested/no TNFα), a second control (no formulation tested but with TNFα) or one of three dilutions of zinc oxide formulation in combination with TNFα. The photos were stained for Zonula Occludens-1, which is red in original color.

FIG. 27 shows data from TER assay experiments described in Example 5. The y-axis refers to TER measured as a percent of the initial value measured. The x-axis refers to a first control (no formulation, no TNFα), a second control (no formulation but with TNFα) and the three dilutions of zinc citrate formulation in combination with TNFα tested.

FIG. 28 shows data from experiments assessing paracellular permeability to FITC-dextran described in Example 5. The y-axis refers to FD-4 Relative fluorescence units measured. The x-axis refers to time point when measurements were taken over a 48-hour period. The data in the graph is for a first control (no formulation tested/no TNFα), a second control (no formulation tested but with TNFα) and the three dilutions of zinc citrate formulation in combination with TNFα.

FIG. 29 shows data from TER assay experiments described in Example 5. The y-axis refers to TER measured as a percent of the initial value measured. The x-axis refers to a first control (no formulation, no TNFα), a second control (no formulation but with TNFα) and the four percentages of zinc oxide-zinc citrate-arginine aqueous solution formulation in combination with TNFα tested.

FIG. 30 shows data from TER assay experiments described in Example 5. The y-axis refers to TER measured as a percent of the initial value measured. The x-axis refers to a first control (no formulation, no TNFα), a second control (no formulation but with TNFα) and the three dilutions of zinc oxide-zinc citrate-arginine dentifrice formulation in combination with TNFα tested.

FIG. 31 shows data from experiments assessing paracellular permeability to FITC-dextran described in Example 5. The y-axis refers to FD-4 Relative fluorescence units measured. The x-axis refers to time point when measurements were taken over a 48-hour period. The data in the graph is for a first control (no formulation tested/no TNFα), a second control (no formulation tested but with TNFα) and the four percentages of the zinc oxide-zinc citrate-arginine aqueous solution formulation in the sample tested together with TNFα.

FIG. 32 shows data from experiments assessing paracellular permeability to FITC-dextran described in Example 5. The y-axis refers to FD-4 Relative fluorescence units measured. The x-axis refers to time point when measurements were taken over a 48-hour period. The data in the graph is for a first control (no formulation tested/no TNFα), a second control (no formulation tested but with TNFα) and the three dilutions of the zinc oxide-zinc citrate-arginine dentifrice formulation tested together with TNFα.

FIG. 33 shows photographs of immunofluorescence of tight junction proteins from experiments described in Example 5. The photos are of either cells treated with a first control (no formulation tested/no TNFα), a second control (no formulation tested but with TNFα) or one of three percentages of the zinc oxide-zinc citrate-arginine aqueous solution formulation in the sample tested together with TNFα. The photos in the top row were stained for Occludin, which is green in original color. The photos in the bottom row were stained for Zonula Occludens-1, which is red in original color.

FIG. 34 shows photographs of immunofluorescence of tight junction proteins from experiments described in Example 5. The photos are of either cells treated with a first control (no formulation tested/no TNFα), a second control (no formulation tested but with TNFα) or one of three dilutions of the zinc oxide-zinc citrate-arginine dentifrice formulation tested together with TNFα. The photos in the top row were stained for Occludin, which is green in original color. The photos in the bottom row were stained for Zonula Occludens-1, which is red in original color.

FIG. 35 shows data from experiments described in Example 6 testing inhibition of P. gingivalis collagenase activity by a negative control, a positive control and three dilutions of zinc oxide formulations. The y-axis refers to Relative fluorescence measured which corresponds to the amount of a labeled collagen substrate processed by collagenase from P. gingivalis. The x-axis refers to time point when measurements were taken over a 5-hour period.

FIG. 36 shows data from experiments described in Example 6 testing effect of a negative control and four dilutions of zinc oxide formulation on invasion of an epithelial cell monolayer by P. gingivalis.

FIG. 37 shows data from experiments described in Example 6 demonstrating testing zinc oxide inhibition of hemolytic activity of P. gingivalis. A first control (no P. gingivalis, no zinc oxide, but with SDS), a second control (no P. gingivalis, no zinc oxide, no SDS), a third control (P. gingivalis but no zinc oxide and no SDS), and three dilutions of zinc oxide together with P. gingivalis but no SDS were tested to measure effect of zinc oxide on hemolytic activity of P. gingivalis on red blood cells from sheep.

FIG. 38 shows data from experiments described in Example 6 testing inhibition of P. gingivalis collagenase activity by a negative control, a positive control and three dilutions of zinc citrate formulations. The y-axis refers to Relative fluorescence measured which corresponds to the amount of a labeled collagen substrate processed by collagenase from P. gingivalis. The x-axis refers to time point when measurements were taken over a 5-hour period.

FIG. 39 shows data from experiments described in Example 6 testing effect of a negative control and four dilutions of zinc citrate formulation on invasion of an epithelial cell monolayer by P. gingivalis.

FIG. 40 shows data from experiments described in Example 6 demonstrating testing zinc oxide inhibition of hemolytic activity of P. gingivalis. A first control (no P. gingivalis, no zinc oxide, but with SDS), a second control (no P. gingivalis, no zinc citrate, no SDS), a third control (P. gingivalis but no zinc citrate and no SDS), and three dilutions of zinc citrate together with P. gingivalis but no SDS were tested to measure effect of zinc citrate on hemolytic activity of P. gingivalis on red blood cells from sheep.

FIG. 41 shows data from experiments described in Example 6 testing inhibition of P. gingivalis collagenase activity by a negative control, a positive control and samples containing three different percentages of a zinc oxide-zinc citrate-arginine formulation. The y-axis refers to Relative fluorescence measured which corresponds to the amount of a labeled collagen substrate processed by collagenase from P. gingivalis. The x-axis refers to time point when measurements were taken over a 5-hour period.

FIG. 42 shows data from experiments described in Example 6 testing effect of a negative control and samples containing three different percentages of a zinc oxide-zinc citrate-arginine formulation on invasion of an epithelial cell monolayer by P. gingivalis.

FIG. 43 shows data from experiments described in Example 6 demonstrating testing inhibition of hemolytic activity of P. gingivalis by a zinc oxide-zinc citrate-arginine formulation. A first control (no P. gingivalis, no zinc oxide-zinc citrate-arginine formulation, but with SDS), a second control (no P. gingivalis, no zinc oxide-zinc citrate-arginine formulation, no SDS), a third control (P. gingivalis but no zinc oxide-zinc citrate-arginine formulation and no SDS), and samples having three different percentages of zinc oxide-zinc citrate-arginine formulation together with P. gingivalis but no SDS were tested to measure effect of zinc oxide-zinc citrate-arginine on hemolytic activity of P. gingivalis on red blood cells from sheep.

FIG. 44 shows data from experiments described in Example 7 to test inhibition of protease activity induced by Aggregatibacter actinomycetemcomitans (Aa) by a composition comprising a combination of zinc oxide-zinc citrate-arginine (a DZA composition). Human cell samples treated with a fluoride toothpaste composition, a fluoride toothpaste composition together with Aa, a composition comprising a DZA composition, and a DZA composition together with Aa and protease activity was measured.

FIG. 45 shows photographic data from experiments described in Example 7 comparing the effect of a DZA composition on cells contacted with Aa. Nuclei and actin in cells were stained with DAPI and Phalloidin, respectively following treatment with Aa or Aa plus a DZA composition. Untreated control cells were also stained. In original color photos, nuclei stain blue and actin stains green.

FIGS. 46A, 46B and 46C show data from experiments described in Example 8 testing the effects of a zinc oxide-zinc citrate-arginine (DZA) in aqueous solution, DZA in a dentifrice, and regular fluoride dentifrice on collagen degradation by P. gingivalis. A value of 100% was assigned to the degradation caused by P. gingivalis in the absence of compounds. Results are expressed as the means±SD of triplicate assays.

FIG. 47 show data from experiments described in Example 8 testing the time- and dose-dependent effects of the DZA aqueous solution, DZA dentifrice, a regular fluoride dentifrice and a negative control on gingival keratinocyte tight junction integrity, as determined by monitoring TER. A 100% value was assigned to the TER values at time 0. The y-axis shows TER measurements as a percent of the initial value. The x axis refers to control plus three dilutions for each formulation tested. Data is provided for four time points. Results are expressed as the means±SD of triplicate assays from three independent experiments. *, significant increase (p<0.001) compared to untreated control cells. φ, significant increase (p<0.001) compared to the regular fluoride dentifrice.

FIGS. 48A, 48B and 48C show data from experiments described in Example 8 testing time- and dose-dependent effects of the DZA aqueous solution (FIG. 48A), DZA dentifrice (FIG. 48B), and regular fluoride dentifrice (FIG. 48C) on the paracellular permeability of gingival keratinocytes to FITC-dextran 4 (FD-4). Control plus three dilutions for each formulation tested. The y-axis shows FD-4 measured as relative fluorescence units as a measure of paracellular permeability. The x-axis shows multiple time points over 48-hour period when data was collected. Results are expressed as the means±SD of triplicate assays from three independent experiments. *, significant decrease (p<0.001) compared to untreated control cells.

FIG. 49 show photographic data from immunofluorescence staining experiments described in Example 8. Immunofluorescence staining of the tight junction proteins occludin and zonula occludens-1 was done using gingival keratinocytes treated for 48 h with DZA aqueous solution, DZA dentifrice, and regular fluoride dentifrice. The top row shows immunostaining of occludin (which stains as the color green in original photo) and the bottom row shows immunostaining of zonula occludens-1 (which stains as the color red in original photo) for control cells, and cells treated with two dilutions of either DZA aqueous solution, DZA Arginine dentifrice, and regular fluoride dentifrice.

FIG. 50 shows data from experiments described in Example 8 testing time- and dose-dependent protective effects of DZA aqueous solution, DZA dentifrice, and regular fluoride dentifrice against P. gingivalis-mediated damage of gingival keratinocyte tight junction integrity as determined by monitoring TER values. A 100% value was assigned to the TER values at time 0. The y-axis shows TER measurements as a percent of the initial value. The x axis refers to the control (no formulation/no P. gingivalis), P. gingivalis challenge (P. gingivalis but no formulation) and three dilutions for each formulation tested in combination with P. gingivalis. Data is provided for four time points. Results are expressed as the means±SD of triplicate assays from three independent experiments. *, significant increase (p<0.001) compared to P. gingivalis-infected cells not treated with compounds. Φ, significant decrease (p<0.001) compared to non-stimulated control cells. φ, significant increase (p<0.001) compared to the regular fluoride dentifrice.

FIGS. 51A, 51B and 51C show data from experiments described in Example 8 testing the protective effects of DZA aqueous solution (FIG. 51A), DZA dentifrice (FIG. 51B), and regular fluoride dentifrice (FIG. 51C) on the paracellular permeability of gingival keratinocytes to FITC-dextran 4 (FD-4) compromised by P. gingivalis. Control, P. gingivalis challenge and three dilutions for each formulation in combination with P. gingivalis were tested. The y-axis shows FD-4 measured as relative fluorescence units as a measure of paracellular permeability. The x-axis shows multiple time points over 48-hour period when data was collected. Results are expressed as the means±SD of triplicate assays from three independent experiments. *, significant decrease (p<0.001) compared to P. gingivalis-stimulated cells.

FIG. 52 show photographic data from immunofluorescence staining experiments described in Example 8 in which cells were treated with P. gingivalis (MOI=10⁴) in the absence and presence of DZA aqueous solution, DZA dentifrice, or regular fluoride dentifrice. Immunofluorescence staining of the tight junction proteins occludin and zonula occludens-1 was done using gingival keratinocytes treated for 48 h. The top row shows immunostaining of occludin (which stains as the color green in the original photo) and the bottom row shows immunostaining of zonula occludens-1 (which stains as the color red in the original photo) for control cells, P. gingivalis challenge and cells treated with two dilutions of either DZA aqueous solution, DZA Arginine dentifrice, and regular fluoride dentifrice in combination with P. gingivalis.

FIGS. 53A, 53B and 53C show data from experiments described in Example 8 testing the DZA aqueous solution (FIG. 53A), DZA dentifrice (FIG. 53B) and regular fluoride dentifrice (FIG. 53C) on the invasion of a gingival keratinocyte barrier by P. gingivalis. The x-axis shows control and three dilutions of formulation. Results are expressed as the means±SD of triplicate assays from three independent experiments. *, significant decrease (p<0.001) compared to P. gingivalis-infected cells not treated with any of the formulations.

FIG. 54 contains data from experiments described in Example 13 showing time- and dose-dependent protective effects of the zinc oxide, zinc citrate and arginine aqueous solution, the zinc oxide, zinc citrate and arginine dentifrice, and the regular fluoride dentifrice against TNF-α-mediated damage of gingival keratinocyte tight junction integrity as determined by monitoring TER values. A 100% value was assigned to the TER values at time 0. Results are expressed as the means±SD of triplicate assays from three independent experiments. *, significant increase (p<0.001) compared to TNF-α-treated cells not treated with compounds. Φ, significant decrease (p<0.001) compared to non-stimulated control cells. φ, significant increase (p<0.001) compared to the regular fluoride dentifrice.

FIGS. 55A, 55B and 55C contain data from experiments described in Example 13 showing protective effects of the zinc oxide, zinc citrate and arginine aqueous solution (FIG. 55A), the zinc oxide, zinc citrate and arginine dentifrice (FIG. 55B), and the regular fluoride dentifrice (FIG. 55C) on the FITC-dextran paracellular transport through the gingival keratinocyte barrier compromised by TNF-α. Results are expressed as the means±SD of triplicate assays from three independent experiments. *, significant decrease (p<0.001) compared to TNF-α-treated cells.

FIG. 56 shows photographs of immunofluorescence staining of the tight junction proteins from experiments described in Example 13. The tight junction proteins occludin and zonula occludens-1 (ZO-1) in gingival keratinocytes were stained following a 48-h treatment with TNF-α (100 ng/ml) in the absence and presence of the zinc oxide, zinc citrate and arginine aqueous solution, the zinc oxide, zinc citrate and arginine dentifrice, or the regular fluoride dentifrice in the indicated dilutions. The photos in the top row were stained for Occludin, which is green in original color. The photos in the bottom row were stained for Zonula Occludens-1, which is red in original color.

FIGS. 57A and 57B contain data from experiments described in Example 13 showing effects of the zinc oxide, zinc citrate and arginine aqueous solution at indicated dilutions on the proliferation of the gingival keratinocytes in the absence (FIG. 57A) and presence of TNF-α (FIG. 57B). Results are expressed as the means±SD of triplicate assays from three independent experiments. *, significant increase (p<0.001) compared to control cells.

FIGS. 58A and 58B contain data from experiments described in Example 13 showing effects of the zinc oxide, zinc citrate and arginine dentifrice at indicated dilutions on the proliferation of the gingival keratinocytes in the absence (FIG. 58A) and presence of TNF-α (FIG. 58B). Results are expressed as the means±SD of triplicate assays from three independent experiments. *, significant increase (p<0.001) compared to control cells.

FIGS. 59A and 59B contain data from experiments described in Example 13 showing effects of the regular fluoride dentifrice at indicated dilutions on the proliferation of gingival keratinocytes in the absence (FIG. 59A) and presence of TNF-α (FIG. 59B). Results are expressed as the means±SD of triplicate assays from three independent experiments. *, significant increase (p<0.001) compared to control cells.

FIGS. 60A, 60B and 60C contain data from experiments described in Example 13 showing effects of the zinc oxide, zinc citrate and arginine aqueous solution at indicated dilutions (FIG. 60A), the zinc oxide, zinc citrate and arginine dentifrice at indicated dilutions (FIG. 60B), and the regular fluoride dentifrice at indicated dilutions (FIG. 60C) on gingival keratinocyte migration. Results are expressed as the means±SD of triplicate assays from three independent experiments. *, significant increase (p<0.001) compared to control cells.

### DETAILED DESCRIPTION

Tissue that functions as a barrier includes oral tissue, for example soft oral tissue such as oral epithelial tissue. Oral epithelial barrier tissue includes gingival epithelial barrier tissue. Enhancing the integrity and function of such tissue promotes overall health by providing a more effective physical barrier against microorganisms, thereby protecting underlying connective tissue. The physical barrier provided by the tissue which functions as a barrier is part of the innate immune system and innate immunity is improved, maintained, protected, repaired and/or restored by methods and compositions that improve, maintain, protect, repair and/or restore tissue integrity of tissue that functions as the physical barrier and by repair of damage to and healing of wounds to such tissue. Oral immunity is thus improved, maintained, protected, repaired and/or restored by methods and compositions that improve, maintain, protects, repair and/or restore the tissue integrity of oral tissue that functions as the physical barrier and by repair of damage to and healing of wounds to such tissue. The physical barrier provided by the oral tissue which functions as a barrier is part of the innate immune system that serves as a first line of defense against oral pathogens.

The integrity of tissue with barrier function, such as soft oral tissue, including for example oral epithelial tissue such as gingival epithelial tissue, is enhanced by improving the function of cell junctions and maintaining the seal of the tight junctions so that the barrier more effectively blocks the pathway to bacteria and toxins. Enhancing the integrity of the tissue with barrier function protects the tissue from deleterious effects on function caused by pathogenic bacteria, for example perio-pathogenic bacteria. Proteases from pathogenic bacteria such as perio-pathogenic bacteria can reduce the integrity and effectiveness of tissue with barrier function. Pathogenic bacteria such as perio-pathogenic bacteria can infiltrate the barrier formed by the cells if the tissue that functions as a barrier, introducing the pathogenic bacteria to underlying tissue and systemically. Pathogenic bacteria such as perio-pathogenic bacteria have hemolytic activity that reduces the integrity and effectiveness of tissue with barrier function. In addition, pathogenic bacteria such as perio-pathogenic bacteria can induce production of proteases such as MMPs in the cells of the host and such proteases further compromise and reduce the integrity of the integrity and effectiveness of tissue with barrier function. Pathogenic bacteria may be Gram-negative or Gram-positive bacteria. Pathogenic bacteria may be anaerobic or aerobic. For example, pathogenic bacteria include Gram-negative anaerobic bacteria. Examples of pathogenic bacteria include Porphyromonas gingivalis, Actinobacillus actinomycetemcomitans, Eikenella corrodens, Prevotella intermedia, Fusobacterium nucleatum, Tannerella forsythia, Treponema denticola, Campylobacter rectus, Campylobacter gracilis, Streptococcus mutans, Streptococcus sobrinus, Streptococcus sanguis, Streptococcus oralis, Actinomyces israelii, Chlamydia pneumoniae, Porphyromonas cangingivalis, Fusobacterium necrophorum, and Streptococcus constellatus.

Deleterious effects to oral health also arise from damage caused by the host's immune response against pathogens and pathogen products. The presence of pathogenic bacteria such as perio-pathogenic bacteria, induces immune responses in the host which includes increased levels of proinflammatory cytokines which may include, among others, PGE2, IL-β, IL-6, IL-8, GM-CSF and TNF-α. The integrity and effectiveness of tissue with barrier function is reduced or compromised by the presence of the proinflammatory cytokines. Inflammation further damages the tissue which functions as a barrier and additionally can damage underlying tissue and bone.

Compositions and methods are also provided which enhance oral tissue's integrity/barrier function. Such compositions and methods improve the function of cell junctions and maintaining the seal of the tight junctions, which include specialized transmembrane proteins that regulate transepithelial permeability, so that the barrier more effectively blocks bacteria and toxins from crossing the pathway that selectively allows movement of molecules such as water and nutrients across the barrier. Such compositions and methods improve the physical structure and function of the tight junctions, more effectively sealing the paracellular space. Improvement in the structure and function of the intercellular tight junction renders the barrier more effective.

Compositions and methods are provided which enhance oral tissue's integrity/barrier function and thereby render such tissue more resistant to challenges by pathogenic bacteria, such as periopathogenic bacteria. For examples, compositions and methods are provided which enhance oral tissue's integrity/barrier function and thereby render such tissue more resistant to challenges by pathogenic bacteria, such as periopathogenic bacteria, including for example P. gingivalis and A. actinomycetemcomitans. Such compositions and methods prevent or reduce the negative effects that the presence of such pathogenic bacteria have on the transepithelial permeability of the barrier provided by the tissue. Such compositions and methods prevent or reduce the negative effects that the presence of such pathogenic bacteria have on the tight junctions and the seal of the paracellular space that the tight junctions provide. Improvement in the structure and function of the intercellular tight junction renders the barrier more effective when challenged by the presence of such pathogenic bacteria.

Compositions and methods are provided which enhance oral tissue's integrity/barrier function and thereby render such tissue more resistant to the negative effects caused by the presence of proinflammatory cytokines, which may include for example PGE2, IL-β, IL-6, IL-8, GM-CSF and TNF-α, among others. Such compositions and methods prevent or reduce the negative effects that the presence of such proinflammatory cytokines have on the transepithelial permeability of the barrier provided by the tissue. Such compositions and methods prevent or reduce the negative effects that the presence of such proinflammatory cytokines have on the tight junctions and the seal of the paracellular space that the tight junctions provide. Improvement in the structure and function of the intercellular tight junction renders the barrier more effective including when such proinflammatory cytokines are present such as when induced as part of an immune response against pathogenic bacteria or factors produced by pathogenic bacteria.

Compositions and methods are provided which enhance oral tissue's integrity/barrier function, rendering such tissue more resistant to challenges by virulence factors generated by pathogenic bacteria, such as for example destructive enzymes that degrade tissue structure, cause lysis of cells and promote invasion of the barrier by pathogenic bacteria. For example, compositions and methods are provided which enhance oral tissue's integrity/barrier function, rendering such tissue more resistant to challenges by virulence factors generated by pathogenic bacteria such as for example destructive enzymes that degrade tissue structure, cause lysis of cells and promote invasion of the barrier by pathogenic bacteria such for example perio-pathogenic bacteria that may include P. gingivalis, A. actinomycetemcomitans and others. Proteases from pathogenic bacteria such as perio-pathogenic bacteria can reduce the integrity and effectiveness of tissue with barrier function. The compositions and methods that are provided inhibit proteases from such pathogenic bacteria, thereby preventing a reduction in the integrity and effectiveness of the tissue that provides barrier function. The compositions and methods that are provided inhibit the invasion of such pathogenic bacteria across tissue that provides barrier function. The compositions and methods that are provided inhibit the hemolytic activity that such pathogenic bacteria have, thereby preventing a reduction in the integrity and effectiveness of the tissue that provides barrier function.

Compositions and methods are provided which enhance oral tissue's integrity/barrier function by inhibiting production of destructive enzymes by host cells in response the presence of pathogenic bacteria. For example, compositions and methods are provided which enhance oral tissue's integrity/barrier function by inhibiting production of destructive enzymes by host cells in response the presence of pathogenic bacteria, such for example perio-pathogenic bacteria that may include P. gingivalis, A. actinomycetemcomitans and others. Proteases such as MMPs which are produced by host cells in response to the presence of such pathogenic bacteria can reduce the integrity and effectiveness of tissue with barrier function. The compositions and methods that are provided inhibit these host cell proteases induced by pathogenic bacteria, thereby preventing a reduction in the integrity and effectiveness of the tissue that provides barrier function.

Compositions and methods are provided that promote restoration and repair of oral tissue's integrity/barrier function. Compositions and methods are provided that repair damage to tissue that functions as a barrier. Compositions and methods are provided that promote wound healing. Compositions and methods are provided that promote keratinocyte proliferation and migration. The deleterious effects of proinflammatory cytokines such as TNF-α result in damage to the keratinocyte barrier and the compositions and methods promote repair and wound healing of such damage by promoting cell proliferation and migration which repairs the damage and restores an intact keratinocyte barrier. The compositions and methods are provided which repairs the damage caused by TNF-α and other factors brought about by presence of pathogenic bacteria, the destructive enzymes from the pathogenic bacteria and from host cells, such as MMPs, in response the presence of such pathogenic bacteria and the factors involved in the inflammatory response induced by the presence of pathogenic bacteria. The compositions and methods restore the integrity and effectiveness of the tissue that provides barrier function by promoting cell proliferation and migration, resulting in effective wound healing. Compositions and methods provided herein may provide benefit to groups of patients vulnerable to reduced barrier integrity, such as individual who have diabetes.

Improvement in tissue integrity of tissue that functions as a barrier renders the specialized transmembrane proteins of the intercellular tight junction more effective at regulating transepithelial permeability.

Improvement in tissue integrity of tissue that functions as a barrier provides improved paracellular permeability function of the intercellular tight junction

Protecting and maintaining tissue integrity of tissue that functions as a barrier renders the barrier more effective against pathogenic bacteria. A barrier more effective against pathogenic bacteria is more effective at regulating transepithelial permeability in the presence of pathogenic bacteria and/or does not undergo a reduction in paracellular permeability function of the intercellular tight junction the presence of pathogenic bacteria.

Protecting and maintaining tissue integrity of tissue that functions as a barrier renders the barrier more effective against deleterious effects of proinflammatory cytokines such as PGE2, IL-β, IL-6, IL-8, GM-CSF and TNF-α. A barrier more effective against deleterious effects of proinflammatory cytokines is more effective at regulating transepithelial permeability in the presence of pathogenic bacteria and/or does not undergo a reduction in paracellular permeability function of the intercellular tight junction the presence of pathogenic bacteria.

Protecting and maintaining tissue integrity of tissue that functions as a barrier renders the barrier more effective against factors released by or otherwise associated with pathogenic bacteria such as proteases of pathogenic bacteria. A barrier more effective against factors released by or otherwise associated with pathogenic bacteria is more effective at regulating transepithelial permeability in the presence of factors released by or otherwise associated with pathogenic bacteria such as proteases of pathogenic bacteria and/or does not undergo a reduction in paracellular permeability function of the intercellular tight junction in the presence of factors released by or otherwise associated with pathogenic bacteria such as proteases of pathogenic bacteria.

Protecting and maintaining tissue integrity of tissue that functions as a barrier may include inhibiting factors released by or otherwise associated with pathogenic bacteria such as proteases of pathogenic bacteria.

Protecting and maintaining tissue integrity of tissue that functions as a barrier may include inhibiting tissue invasion by pathogenic bacteria that is facilitated by proteases of pathogenic bacteria.

Protecting and maintaining tissue integrity of tissue that functions as a barrier may include inhibiting cell lysis activity, such as hemolytic activity by pathogenic bacteria.

Protecting and maintaining tissue integrity of tissue that functions as a barrier may include inhibiting induction of protease production, such as MMP production by host cells, and/or host cell protease activity, such as host cell MMP activity in response to the presence of pathogenic bacteria.

Repairing and restoring the keratinocyte barrier damaged by pro-inflammatory cytokines such as TNF-α and others as well as bacterial and host proteases effectively heals the wound to the barrier.

In vitro models can be used in methods of identifying and evaluating compositions useful to improve tissue integrity and barrier function and to assess the effectiveness of compositions to protect and maintain tissue integrity and barrier function in response to pathogens and pathogenic factors as well as pro-inflammatory cytokines that trigger inflammation which leads to compromises in tissue integrity and reduction in barrier function that leads to tissue damage. In vitro models can be used in methods of identifying and evaluating compositions that are useful to protect and maintain tissue integrity and barrier function by inhibiting pathogenic factors such as proteases produced by pathogenic bacteria which can reduce or compromise tissue integrity and barrier function, that are useful to protect and maintain tissue integrity and barrier function by inhibiting invasion of barrier tissue by pathogenic bacteria thereby reducing or compromising tissue integrity and barrier function, and that are useful to protect and maintain tissue integrity and barrier function by inhibiting hemolytic activity of pathogenic bacteria which reduces or compromises tissue integrity and barrier function. In vitro models can be used in methods of identifying and evaluating compositions that are useful to protect and maintain tissue integrity and barrier function by inhibiting protease activity of proteases produced by host cells in response to the presence of pathogenic bacteria which reduces or compromises tissue integrity and barrier function.

Assays for assessing tissue integrity include a trans-epithelial electrical resistance (TER) assay, assays that measure paracellular permeability to FITC-dextran and assays which use immunofluorescence of visualize tight junction proteins. These assays can be used to identify compositions that when present, improve and enhance tissue integrity. These assays can be used to identify compositions that when present, can maintain and protect tissue integrity from the damaging effects of and the damage caused by pathogens and their pathogenic factors as well as to identify compositions that when present, can maintain and protect tissue integrity from the damaging effects of and damage caused by pro-inflammatory cytokines. Additional assays are provided for assessing the protective effect of compositions include assays which assess methods and composition's inhibitory effect on protease activity such as collagenase activity, assays which assess methods and composition's inhibitory effect on epithelial tissue invasion by pathogenic bacteria, and assays which assess methods and composition's inhibitory effect on hemolytic activity. In addition, assays are provided which are useful for assessing the protective effect of methods and compositions from pathogen induced MMPs.

Oral care compositions, such as toothpastes, oral rinses and mouth washes, that comprise one or more zinc ion sources may be useful to enhance and improve tissue integrity and/or to reduce the level of damage to tissue integrity caused by the presence of a bacterial pathogen and/or to reduce the level of damage to tissue integrity caused by the presence of a pro-inflammatory cytokine. Enhancement and improvement of tissue integrity, reduction of the level of damage to tissue integrity caused by the presence of a bacterial pathogen and reduction of the level of damage to tissue integrity caused by the presence of a pro-inflammatory cytokine may be determined, for example, by using the TER assay and/or assays that measure paracellular permeability to FITC-dextran and/or assays which use immunofluorescence of visualize tight junction proteins. Oral care compositions, such as toothpastes, oral rinses and mouth washes, that comprise one or more zinc ion sources may be useful to inhibit collagenase produced by a bacterial pathogen and/or to inhibit invasion of an epithelial cell monolayer and/or to inhibit bacterial pathogen induced hemolysis. Oral care compositions, such as toothpastes, oral rinses and mouth washes, that comprise one or more zinc ion sources may be useful to inhibit the induction of proteases such as MMPs, which are critical virulent factors to degrade gum tissue in perio-etiology. Oral care compositions, such as toothpastes, oral rinses and mouth washes, that comprise one or more zinc ion sources may be useful to repair damaged barrier by promoting cell proliferation and migration. Assays which determine keratinocyte proliferation and migration demonstrate the effectiveness of repairing damage and healing wounds to the barrier, restoring effective function.

A zinc ion source is capable of providing Zn′ ions to the oral cavity, including delivery to a surface in the oral cavity. In some embodiments, the zinc ion source is capable of delivering Zn²⁺ ions to the oral mucosa including the gingival epithelia. In some embodiments, the oral care composition comprises one or more zinc ion sources. In some embodiments, the oral care composition comprises one, two, three, four or more zinc ion sources. Examples of zinc ion sources include zinc chloride, zinc acetate, zinc gluconate, zinc sulphate, zinc fluoride, zinc citrate, zinc lactate, zinc oxide, zinc monoglycerolate, zinc tartrate, zinc pyrophosphate, zinc phosphate, zinc maleate, zinc malate, zinc carbonate, zinc ascorbate, zinc lysine hydrochloride and zinc chloride hydroxide monohydrate (TBZC). In some embodiments, the oral care composition comprises between 1 and 20,000 ppm zinc. Further optionally the composition comprises between 1 and 10,000 ppm, between 1 and 5,000 ppm zinc, between 1 and 2,000 ppm zinc. between 1 and 1,000 ppm zinc, between 1 and 500 ppm zinc, between 1 and 200 ppm zinc, between and 100 ppm zinc, between 1 and 50 ppm zinc, between 1 and 25 ppm zinc, between 1 and 10 ppm, or between 3 and 9 ppm zinc. Further optionally the composition comprises between 4 and 8 ppm zinc. Optionally the composition comprises from 0.0001 to 2.0 weight % zinc. Further optionally the composition comprises from 0.0001 to 1.0 weight % zinc, from 0.0001 to 0.5 weight % zinc, from 0.0001 to 0.2 weight % zinc, from 0.0001 to 0.1 weight % zinc, from 0.0001 to 0.05 weight % zinc, from 0.0001 to 0.02 weight % zinc, from 0.0001 to 0.01 weight % zinc, from 0.0001 to 0.005 weight % zinc, from 0.0001 to 0.0025 weight % zinc, from 0.0001 to 0.001 weight % zinc or between 0.0003 and 0.0009 weight % zinc. Further optionally the composition comprises between 0.0004 and 0.0008 weight % zinc. In some embodiments, the composition delivers between 1 and 10 ppm zinc to the gingival epithelial cells, for example from 3 to 9 ppm zinc or from 4 to 8 ppm zinc. In one embodiment the composition delivers about 4 ppm zinc or about 8 ppm zinc.

Compositions were tested and analyzed for effects in enhancing and improving tissue integrity using TER assays, using assays that measure paracellular permeability to FITC-dextran and using assays which use immunofluorescence of visualize tight junction proteins. These assays were used to measure the effect of pathogenic bacteria and pro-inflammatory cytokines in the presence or absence of the compositions to determine activity to that protects tissue integrity from the damaging effects of and damage caused by pathogens or the damaging effects of and damage caused by pro-inflammatory cytokines. The protective effect of compositions on tissue integrity was also analyzed using assays which assess their inhibitory effect on protease activity such as collagenase activity, assays which assess their inhibitory effect on epithelial tissue invasion, assays which assess their inhibitory effect on hemolytic activity, and assays that assess their protective effect against pathogens and pathogen induced MMPs.

Oral care compositions, particularly tooth pastes that comprise zinc oxide or zinc citrate or a combination of zinc oxide and zinc citrate, optionally in further combination with arginine, were found to enhance and improve tissue integrity using the TER assay, assays that measure paracellular permeability to FITC-dextran and assays which use immunofluorescence of visualize tight junction proteins. Oral care compositions, particularly toothpastes that comprise zinc oxide or zinc citrate or a combination of zinc oxide and zinc citrate, optionally in further combination with arginine, were found to reduce the level of damage to tissue integrity by the presence of a bacterial pathogen as measured using the TER assay, assays that measure paracellular permeability to FITC-dextran and assays which use immunofluorescence of visualize tight junction proteins. Oral care compositions, particularly toothpastes that comprise zinc oxide or zinc citrate or a combination of zinc oxide and zinc citrate, optionally in further combination with arginine, were found to reduce the level of damage to tissue integrity by the presence of a pro-inflammatory cytokine as measured using the TER assay, assays that measure paracellular permeability to FITC-dextran and assays which use immunofluorescence of visualize tight junction proteins. Oral care compositions, particularly toothpastes that comprise zinc oxide or zinc citrate or a combination of zinc oxide and zinc citrate, optionally in further combination with arginine, were found to inhibit collagenase produced by a bacterial pathogen, to inhibit invasion of an epithelial cell monolayer and to inhibit bacterial pathogen induced hemolysis as well as the induction of proteases such as MMPs, which are critical virulent factors to degrade gum tissue in perio-etiology. Oral care compositions, particularly toothpastes that comprise zinc oxide and zinc citrate in combination with arginine, were found to promote keratinocyte proliferation and migration useful in the repair damaged tissue barrier, such as damaged by TNF-α, and restoration of barrier function.

Methods provided herein comprise the step of applying to the oral cavity of an individual, oral care compositions that enhance oral tissue barrier integrity. Methods provided herein comprise the step of applying to the oral cavity of an individual, oral care compositions that protect oral tissue barrier from damage associated with the presence of pathogenic bacteria. Methods provided herein comprise the step of applying to the oral cavity of an individual, oral care compositions that protect oral tissue barrier from damage associated with the presence of proinflammatory cytokines induced by the presence of pathogenic bacteria or factors they may produce. Methods provided herein comprise the step of applying to the oral cavity of an individual, oral care compositions that protect oral tissue barrier by inhibiting proteases, toxins and other factors produced by pathogenic bacteria that compromise tissue integrity. Methods provided herein comprise the step of applying to the oral cavity of an individual, oral care compositions that protect oral tissue barrier by inhibiting invasion of barrier tissue by pathogenic bacteria. Methods provided herein comprise the step of applying to the oral cavity of an individual, oral care compositions that protect oral tissue barrier by inhibiting hemolytic activity of pathogenic bacteria. Methods provided herein comprise the step of applying to the oral cavity of an individual, oral care compositions that protect oral tissue barrier by inhibiting protease activity by proteases produced by host cells in response to the presence of pathogenic bacteria or factors they may produce. Methods provided herein comprise the step of applying to the oral cavity of an individual, oral care compositions that repair damage to the oral tissue barrier produced by the presence of pathogenic bacteria, proteolytic activity of pathogenic bacteria, proteolytic activity of the host and inflammatory responses to the presence of pathogenic bacteria. The damage is repaired by promoting keratinocyte proliferation and migration, restoring an effective intact keratinocyte barrier. The oral tissue barrier includes the oral epithelial tissue barrier such as gingival epithelial barrier tissue. Enhancing or improving tissue barrier integrity and protecting the oral tissue barrier from damage associated with pathogenic bacteria and toxins they produce promotes good oral health.

Oral care compositions useful in the methods provided comprise zinc oxide or zinc citrate or a combination of zinc oxide and zinc citrate, optionally in further combination with arginine and/or one or more of alanine, asparagine, cysteine, glutamine, glycine, isoleucine, leucine, methionine, phenylalanine, proline, serine, threonine, tryptophan, tyrosine, valine and amino acids having an isoelectric point of pH 5.0 to 7.0. Oral care compositions include toothpastes, mouthwashes and oral rinses that comprise zinc oxide or zinc citrate or a combination of zinc oxide and zinc citrate, optionally in further combination with arginine and/or one or more of alanine, asparagine, cysteine, glutamine, glycine, isoleucine, leucine, methionine, phenylalanine, proline, serine, threonine, tryptophan, tyrosine, valine and amino acids having an isoelectric point of pH 5.0 to 7.0 in amounts effective in such methods.

In some embodiments the oral care compositions comprise zinc oxide. In some embodiments, the total concentration of zinc oxide in the composition is from 0.2 weight % to 5 weight %, or from 0.5 weight % to 2.5 weight % or from 0.8 weight % to 2 weight %, or about 1.5 weight %, based on the total weight of the composition. In some embodiments, arginine may further be present. In some embodiments, the molar ratio of arginine to zinc oxide is from 0.05:1 to 10:1. In some embodiments, the composition comprises zinc oxide in an amount of from 0.5 weight % to 1.5 weight % and in some embodiments in an amount of from 0.25 weight % to 0.75 weight % based on the total weight of the composition. In some embodiments, the composition may comprise zinc oxide in an amount of from 0.75 weight % to 1.25 weight % and in some embodiments in an amount of from 0.4 weight % to 0.6 weight %, based on the total weight of the composition. In some embodiments, the composition comprises zinc oxide in an amount of about 1 weight % based on the total weight of the composition. In some embodiments, the composition comprises zinc oxide in an amount of about 0.5 weight %, based on the total weight of the composition. In some embodiments, zinc oxide may be present in an amount of from 0.75 to 1.25 wt % (e.g., 1.0 wt. %) based on the weight of the oral care composition. In some embodiments, zinc oxide is in an amount of from 0.25 to 1.0 wt % (e.g. 0.25 to 0.75 wt. %, or 0.5 wt. %) based on the weight of the oral care composition. In some embodiments, the zinc oxide is about 1.0 wt %. In some embodiments, the zinc oxide is about 0.5 wt %.

In some embodiments the oral care compositions comprise zinc citrate. In some embodiments, the total concentration of zinc citrate in the composition is from 0.2 weight % to 5 weight %, or from 0.5 weight % to 2.5 weight % or from 0.8 weight % to 2 weight %, or about 1.5 weight %, based on the total weight of the composition. In some embodiments, arginine may further be present. In some embodiments, the molar ratio of arginine to zinc citrate is from 0.05:1 to 10:1. In some embodiments, the composition comprises zinc citrate zinc oxide in an amount of from 0.5 weight % to 1.5 weight % and in some embodiments in an amount of from 0.25 weight % to 0.75 weight %, based on the total weight of the composition. In some embodiments, the composition may comprise zinc citrate in an amount of from 0.75 weight % to 1.25 weight % and in some embodiments in an amount of from 0.4 weight % to 0.6 weight %, based on the total weight of the composition. In some embodiments, the composition comprises zinc citrate in an amount of about 1 weight %, based on the total weight of the composition. In some embodiments, the composition comprises zinc citrate in an amount of about 0.5 weight %, based on the total weight of the composition. In some embodiments, zinc citrate may be present in an amount of from 0.75 to 1.25 wt % (e.g., 1.0 wt. %) based on the weight of the oral care composition. In some embodiments, zinc citrate is in an amount of from 0.25 to 1.0 wt % (e.g. 0.25 to 0.75 wt. %, or 0.5 wt. %) based on the weight of the oral care composition. In some embodiments, the zinc citrate is about 1.0 wt %. In some embodiments, the zinc citrate is about 0.5 wt %.

In some embodiments the oral care compositions comprise a combination of zinc oxide and zinc citrate in the various amounts described above. In some embodiments the oral care compositions comprise a combination of zinc oxide and zinc citrate in which the ratio of zinc oxide to zinc citrate is from 1.5:1 to 4.5:1, 1.5:1 to 4:1, 1.7:1 to 2.3:1, 1.9:1 to 2.1:1, or about 2:1. Also, the corresponding molar ratios based on these weight ratios can be used. In some embodiments, the total concentration of zinc salts in the composition is from 0.2 weight % to 5 weight %, or from 0.5 weight % to 2.5 weight % or from 0.8 weight % to 2 weight %, or about 1.5 weight %, based on the total weight of the composition. In some embodiments, the molar ratio of arginine to total zinc salts is from 0.05:1 to 10:1. In some embodiments, the composition comprises zinc oxide in an amount of from 0.5 weight % to 1.5 weight % and zinc citrate in an amount of from 0.25 weight % to 0.75 weight %, based on the total weight of the composition. In some embodiments, the composition may comprise zinc oxide in an amount of from 0.75 weight % to 1.25 weigh % and zinc citrate in an amount of from 0.4 weight % to 0.6 weight %, based on the total weight of the composition. In some embodiments, the composition comprises zinc oxide in an amount of about 1 weight % and zinc citrate in an amount of about 0.5 weight %, based on the total weight of the composition. In some embodiments, zinc oxide may be present in an amount of from 0.75 to 1.25 wt % (e.g., 1.0 wt. %) the zinc citrate is in an amount of from 0.25 to 1.0 wt % (e.g. 0.25 to 0.75 wt. %, or 0.5 wt. %) and based on the weight of the oral care composition. In some embodiments, the zinc citrate is about 0.5 wt %. In some embodiments, the zinc oxide is about 1.0 wt %.

In some embodiments the zinc oxide (ZnO) particles may have an average particle size of from 1 to 7 microns. In some embodiments, the ZnO particles have an average particle size of 5 microns or less. In some embodiments, suitable zinc oxide particles for oral care compositions have, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns. Zinc oxide may have a particle size which is a median particle size. Suitable particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns. In another aspect, that particle size is an average (mean) particle size. In an embodiment, the mean particle comprises at least 5%, at least 10%, at least 15%, at least 20%, at least 25%, at least 30%, at least 35%, or at least 40% of the total metal oxide particles in an oral care composition of the invention. The particle may be present in an amount of up to 5% by weight, based on the total weight of the oral care composition, for example in an amount of from 0.5 to 5% by weight, preferably of up to 2% by weight, more preferably from 0.5 to 2% by weight, more preferably from 1 to 2% by weight, or in some embodiment from 2.5 to 4.5% by weight, being based on the total weight of the oral care composition. In some embodiments, the source of zinc oxide particles and/or the form they may be incorporated into the oral care composition in is selected from one or more of a powder, a nanoparticle solution or suspension, or encapsulated in a polymer or bead. Zinc oxide particles may be selected to achieve occlusion of dentin particles. Particle size distribution may be measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the metal oxide. It will be understood that other methods of measuring particle size are known in the art, and based on the disclosure set forth herein, the skilled artisan will understand how to calculate median particle size, mean particle size, and/or particle size distribution of metal oxide particles.

Oral care compositions optionally further comprise arginine or a salt thereof. In some embodiments, the arginine is L-arginine or a salt thereof. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. In some embodiments, the arginine in partially or wholly in salt form such as arginine phosphate, arginine hydrochloride or arginine bicarbonate. In some embodiments, the arginine is present in an amount corresponding to 0.1% to 15%, e.g., 0.1 wt % to 10 wt %, e.g., 0.1 to 5 wt %, e.g., 0.5 wt % to 3 wt % of the total composition weight, about e.g., 1%, 1.5%, 2%, 3%, 4%, 5%, or 8%, wherein the weight of the arginine is calculated as free form. In some embodiments the arginine is present in an amount corresponding to about 0.5 wt. % to about 20 wt. % of the total composition weight, about 0.5 wt. % to about 10 wt. % of the total composition weight, for example about 1.5 wt. %, about 3.75 wt. %, about 5 wt. %, or about 7.5 wt. % wherein the weight of the arginine is calculated as free form. In some embodiments, the arginine is present in an amount of from 0.5 weight % to 10 weight %, or from 0.5 weight % to 3 weight % or from 1 weight % to 2.85 weight %, or from 1.17 weight % to 2.25 weight %, based or from 1.4 weight % to 1.6 weight %, or from 0.75 weight % to 2.9 weight %, or from 1.3 weight % to 2 weight %, or about 1.5 weight %, based on the total weight of the composition. Typically, the arginine is present in an amount of up to 5% by weight, further optionally from 0.5 to 5% by weight, still further optionally from 2.5 to 4.5% by weight, based on the total weight of the oral care composition. In some embodiments, arginine is present in an amount from 0.1 wt. %-6.0 wt. %. (e.g., about 1.5 wt %) or from about 4.5 wt. %-8.5 wt. % (e.g., 5.0%) or from 3.5 wt. %-9 wt. % or 8.0 wt. %. In some embodiments, the arginine is present in a dentifrice, at for example about 0.5-2 wt. %, e.g., and about 0.8% in the case of a mouthwash.

Oral care compositions optionally further comprise one or more amino acids selected from the group consisting of: alanine, asparagine, cysteine, glutamine, glycine, isoleucine, leucine, methionine, phenylalanine, proline, serine, threonine, tryptophan, tyrosine, valine, and amino acids which have an isoelectric point in range of pH 5.0 to 7.0. The one or more amino acids, which may each be referred to as being neutral, each has a single amino group and carboxyl group or a functional derivative hereof, such as functional derivatives having an altered side chain. Functional derivatives have similar or substantially similar biological and chemical properties. In some embodiments the amino acid is at least partially water soluble and have a pH of less than 7 in an aqueous solution of 1 g/1000 ml at 25° C. In some embodiments, the one or more amino acids include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, and valine. Preferably, the one or more amino acids include asparagine, glutamine, glycine, salts thereof, or combinations thereof, and more preferably in its free form. The one or more amino acids may have an isoelectric point of 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0 in an aqueous solution at 25° C. In some embodiments, the one or more amino acid is present in the amount of from 0.01% to 10%, from 0.05% to 5%, yet more preferably from 0.1% to 2%, alternatively from 0.2% to 3%, by weight of the composition. In some embodiments, such oral care compositions additionally comprise arginine or a salt thereof such as for example L-arginine or a salt thereof in combination with such amino acids. In one example, compositions comprise glutamine. In another example, compositions comprise glutamine and arginine. In another example, compositions comprise asparagine. In another example, compositions comprise asparagine and arginine. In yet another example, compositions comprise glycine. In another example, compositions comprise glycine and arginine. In some embodiments, such oral care compositions that comprise arginine or a salt thereof comprise L-arginine or a salt thereof.

Oral care compositions may optionally further comprise one or more fluoride ion sources present in an amount providing a clinically efficacious amount of soluble fluoride ion to the oral care composition. A fluoride ion source is useful, for example, as an anti-caries agent. Any orally acceptable particulated fluoride ion source can be used, including stannous fluoride, sodium fluoride, potassium fluoride, potassium monofluorophosphate, sodium monofluorophosphate, ammonium monofluorophosphate, sodium fluorosilicate, ammonium fluorosilicate, indium fluoride, amine fluoride such as olaflur (N′-octadecyltrimethylendiamine-N,N,N′-tris(2-ethanol)-dihydrofluoride), ammonium fluoride, titanium fluoride, hexafluorosulfate, and combinations thereof. Fluoride where present may be present at levels of, e.g., about 25 to about 25,000 ppm, for example about 50 to about 5000 ppm, about 750 to about 2,000 ppm for a consumer toothpaste (e.g., 1000-1500 ppm, e.g., about 1000 ppm, e.g., about 1450 ppm), product. In some embodiments, fluoride is present from about 100 to about 1000, from about 200 to about 500, or about 250 ppm fluoride ion. 500 to 3000 ppm. In some embodiments, the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm). In some embodiments, the fluoride source is stannous fluoride. In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. In some embodiments, the fluoride source is sodium fluoride which provides fluoride in an amount from 750-2000 ppm (e.g., about 1450 ppm). In some embodiments, the fluoride source is selected from sodium fluoride and sodium monofluorophosphate and which provides fluoride in an amount from 1000 ppm-1500 ppm. In some embodiments, the fluoride source is sodium fluoride or sodium monofluorophosphate and which provides fluoride in an amount of about 1450 ppm. In some embodiments, stannous fluoride is the only fluoride source. In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. In some embodiment, the stannous fluoride is present in an amount of 0.1 wt. % to 2 wt. % (0.1 wt %-0.6 wt. %) of the total composition weight. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts. In some embodiment, the fluoride source is a fluoride salt present in an amount of 0.1 wt. % to 2 wt. % (0.1 wt %-0.6 wt. %) of the total composition weight (e.g., sodium fluoride (e.g., about 0.32 wt. %) or sodium monofluorophosphate). e.g., 0.3-0.4%, e.g., ca. 0.32% sodium fluoride

The oral care compositions described herein may also comprise one or more further agents such as those typically selected from the group consisting of: abrasives, an anti-plaque agent, a whitening agent, antibacterial agent, cleaning agent, a flavoring agent, a sweetening agent, adhesion agents, surfactants, foam modulators, pH modifying agents, humectants, mouth-feel agents, colorants, tartar control (anti-calculus) agent, polymers, saliva stimulating agent, nutrient, viscosity modifier, anti-sensitivity agent, antioxidant, and combinations thereof.

In some embodiments, the oral care compositions comprise one or more abrasive particulates such as those useful for example as a polishing agent. Any orally acceptable abrasive can be used, but type, fineness, (particle size) and amount of abrasive should be selected so that tooth enamel is not excessively abraded in normal use of the composition. Examples of abrasive particulates may be used include abrasives such sodium bicarbonate, insoluble phosphates (such as orthophosphates, polymetaphosphates and pyrophosphates including dicalcium orthophosphate dihydrate, calcium pyrophosphate, tricalcium phosphate, calcium polymetaphosphate and insoluble sodium polymetaphosphate), calcium phosphate (e.g., dicalcium phosphate dihydrate), calcium sulfate, natural calcium carbonate (CC), precipitated calcium carbonate (PCC), silica (e.g., hydrated silica or silica gels or in the form of precipitated silica or as admixed with alumina), iron oxide, aluminum oxide, aluminum silicate, calcined alumina, bentonite, other siliceous materials, perlite, plastic particles, e.g., polyethylene, and combinations thereof. The natural calcium carbonate abrasive of is typically a finely ground limestone which may optionally be refined or partially refined to remove impurities. The material preferably has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004%) by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8-4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high-water absorption, e.g., at least 25 g/100 g, e.g. 30-70 g/100 g. Examples of commercially available products suitable for use include, for example, Carbolag® 15 Plus from Lagos Industria Quimica. In some embodiments, additional calcium-containing abrasives, for example calcium phosphate abrasive, e.g., tricalcium phosphate, hydroxyapatite or dicalcium phosphate dihydrate or calcium pyrophosphate, and/or silica abrasives, sodium metaphosphate, potassium metaphosphate, aluminum silicate, calcined alumina, bentonite or other siliceous materials, or combinations thereof are used. Examples of silica abrasives include, but are not limited to, precipitated or hydrated silicas having a mean particle size of up to about 20 microns (such as Zeodent 105 and Zeodent 114 marketed by J.M. Huber Chemicals Division, Havre de Grace, Md. 21078); Sylodent 783 (marketed by Davison Chemical Division of W.R. Grace & Company); or Sorbosil AC 43 (from PQ Corporation). In some embodiments, an effective amount of a silica abrasive is about 10-30%, e.g. about 20%. In some embodiments, the acidic silica abrasive Sylodent is included at a concentration of about 2 to about 35% by weight; about 3 to about 20% by weight, about 3 to about 15% by weight, about 10 to about 15% by weight. For example, the acidic silica abrasive may be present in an amount selected from 2 wt. %, 3 wt. %, 4% wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %. Sylodent 783 has a pH of 3.4-4.2 when measured as a 5% by weight slurry in water and silica material has an average particle size of less than 10 microns, e.g., 3-7 microns, e.g. about 5.5 microns. In some embodiments, the silica is synthetic amorphous silica, (e.g., 1%-28% by wt.) (e.g., 8%-25% by wt). In some embodiments, the silica abrasives are silica gels or precipitated amorphous silicas, e.g. silicas having an average particle size ranging from 2.5 microns to 12 microns. Some embodiments further comprise a small particle silica having a median particle size (d50) of 1-5 microns (e.g., 3-4 microns) (e.g., about 5 wt. % Sorbosil AC43 from PQ Corporation Warrington, United Kingdom). The composition may contain from 5 to 20 wt % small particle silica, or for example 10-15 wt %, or for example 5 wt %, 10 wt %, 15 wt % or 20 wt % small particle silica. In some embodiments, 20-30 wt % of the total silica in the composition is small particle silica (e.g., having a median particle size (d50) of 3-4 microns and wherein the small particle silica is about 5 wt. % of the oral care composition. In some embodiments, silica is used as a thickening agent, e.g., particle silica. In some embodiments, the composition comprises calcium carbonate, such as precipitated calcium carbonate high absorption (e.g., 20% to 30% by weight of the composition or, 25% precipitated calcium carbonate high absorption), or precipitated calcium carbonate—light (e.g., about 10% precipitated calcium carbonate—light) or about 10% natural calcium carbonate.

In some embodiments, the oral care compositions comprise a whitening agent, e.g., a selected from the group consisting of peroxides, metal chlorites, perborates, percarbonates, peroxyacids, hypochlorites, hydroxyapatite, and combinations thereof. Oral care compositions may comprise hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate or hydrogen peroxide polymer complexes such as hydrogen peroxide-polyvinyl pyrrolidone polymer complexes.

In some embodiments, the oral care compositions comprise an effective amount of one or more antibacterial agents, for example comprising an antibacterial agent selected from halogenated diphenyl ether (e.g. triclosan), triclosan monophosphate, herbal extracts and essential oils (e.g., rosemary extract, tea extract, magnolia extract, thymol, menthol, eucalyptol, geraniol, carvacrol, citral, hinokitol, magonol, ursolic acid, ursic acid, morin, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine furanones, bacteriocins, ethyllauroyl arginate, arginine bicarbonate, a Camellia extract, a flavonoid, a flavan, halogenated diphenyl ether, creatine, sanguinarine, povidone iodine, delmopinol, salifluor, metal ions (e.g., zinc salts, stannous salts, copper salts, iron salts), propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nisin preparations, chlorite salts; parabens such as methylparaben or propylparaben and mixtures of any of the foregoing. One or more additional antibacterial or preservative agents may optionally be present in the composition in a total amount of from about 0.01 wt. % to about 0.5 wt. %, optionally about 0.05 wt. % to about 0.1 wt. % or about 0.3%.by total weight of the composition.

In some embodiments, the oral care compositions may comprise at least one bicarbonate salt useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. The one or more additional bicarbonate salts are optionally present in a total amount of about 0.1 wt. % to about 50 wt. %, for example about 1 wt. % to 20 wt. %, by total weight of the composition.

In some embodiments, the oral care compositions also comprise at least one flavorant, useful for example to enhance taste of the composition. Any orally acceptable natural or synthetic flavorant can be used, including without limitation essential oils and various flavoring aldehydes, esters, alcohols, and similar materials, tea flavors, vanillin, sage, marjoram, parsley oil, spearmint oil, cinnamon oil, oil of wintergreen, peppermint oil, clove oil, bay oil, anise oil, eucalyptus oil, citrus oils, fruit oils, sassafras and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or wanning effects. Such ingredients illustratively include menthol, carvone, menthyl acetate, menthyl lactate, camphor, eucalyptus oil, eucalyptol, anethole, eugenol, cassia, oxanone, a-irisone, propenyl guaiethoi, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 0.3 wt. % and in some embodiments in various embodiments from about 0.01 wt. % to about 1 wt. %, from about 0.05 to about 2%, from about 0.1% to about 2.5%, and from about 0.1 to about 0.5% by total weight of the composition.

In some embodiments, the oral care compositions comprise at least one sweetener, useful for example to enhance taste of the composition. Sweetening agents among those useful herein include dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, ethanol, sorbitol, mannitol, xylitol, maltitol, isomalt, aspartame, neotame, saccharin and salts thereof (e.g. sodium saccharin), sucralose, dipeptide-based intense sweeteners, cyclamates, dihydrochalcones, glycerine, propylene glycol, polyethylene glycols, Poloxomer polymers such as POLOXOMER 407, PLURONIC F108, (both available from BASF Corporation), alkyl polyglycoside (APG), polysorbate, PEG40, castor oil, menthol, and mixtures thereof. One or more sweeteners are optionally present in a total amount depending strongly on the particular sweetener(s) selected, but typically 0.005 wt. % to 5 wt. %, by total weight of the composition, optionally 0.005 wt. % to 0.2 wt. %, further optionally 0.05 wt. % to 0.1 wt. % by total weight of the composition.

In some embodiments, the oral care compositions further comprise an agent that interferes with or prevents bacterial attachment, e.g., ethyl lauroyl arginiate (ELA), solbrol or chitosan, as well as plaque dispersing agents such as enzymes (papain, glucoamylase, etc.).

In some embodiments, the oral care compositions also comprise at least one surfactant. Any orally acceptable surfactant, most of which are anionic, cationic, zwitterionic, nonionic or amphoteric, and mixtures thereof, can be used. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate, sodium coconut monoglyceride sulfonate, sodium lauryl sarcosinate, sodium lauryl isoethionate, sodium laureth carboxylate and sodium dodecyl benzenesulfonate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of amides include N-lauryl sarcosine, and the sodium, potassium and ethanolamine salts of N-lauryl, N-myristoyl, or N-palmitoyl sarcosine. Examples of cationic surfactants include derivatives of aliphatic quaternary ammonium compounds having one long alkyl chain containing 8 to 18 carbon atoms such as lauryl trimethylammonium chloride, cetyl pyridinium chloride, cetyl trimethyl ammonium bromide, di-isobutylphenoxyethyldimethylbenzylammonium chloride, coconut alkyltrimethylammonium nitrite, cetyl pyridinium fluoride, and mixtures thereof. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, di alkyl sulfoxides and the like. Others include, for example, non-anionic polyoxyethylene surfactants, such as Polyoxamer 407, Steareth 30, Polysorbate 20, and castor oil; and amphoteric surfactants such as derivatives of aliphatic secondary and tertiary amines having an anionic group such as carboxylate, sulfate, sulfonate, phosphate or phosphonate such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides. In some embodiments, the oral composition includes a surfactant system that is sodium laurel sulfate (SLS) and cocamidopropyl betaine. One or more surfactants are optionally present in a total amount of about 0.01 wt. % to about 10 wt. %, for example, from about 0.05 wt. % to about 5 wt. %, or from about 0.1 wt. % to about 2 wt. %, e.g. 1.5% wt. by total weight of the composition. In some embodiments, the oral composition include an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS); and/or a zwitterionic surfactant, for example a betaine surfactant, for example cocamidopropylbetaine, e.g. in an amount of from about 0.1% to about 4.5% by weight, e.g. 0.5-2% cocamidopropylbetaine. Some embodiments comprise a nonionic surfactant in an amount of from 0.5-5%, e.g. 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof. In some embodiments, the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407. Any of the preceding compositions may further comprise sorbitol, wherein the sorbitol is in a total amount of 10-40% (e.g., about 23%).

In some embodiments, the oral care compositions comprise at least, one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000,000 to 2,500,000, One or more PEGs are optionally present in a total amount of about 0.1 wt. % to about 10 wt. %, for example from about 0.2 wt. % to about 5 wt. %, or from about 0.25 wt. % to about 2 wt. %, by total weight of the composition

In some embodiments, the oral care compositions comprise at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates such as sodium bicarbonate, sesquicarbonates, borates, silicates, bisulfates, phosphates (e.g., monosodium phosphate, trisodium phosphate, monopotassium phosphate, dipotassium phosphate, tribasic sodium phosphate, sodium tripolyphosphate, phosphoric acid), imidazole, sodium phosphate buffer (e.g., sodium phosphate monobasic and disodium phosphate) citrates (e.g. citric acid, trisodium citrate dehydrate), pyrophosphates (sodium and potassium salts) and the like and combinations thereof. One or more pH modifying agents are optionally present in a total amount effective to maintain the composition in an orally acceptable pH range. Compositions may have a pH that is either acidic or basic, e.g., from pH 4 to pH 5.5 or from pH 8 to pH 10. In some embodiments, the amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%), in another embodiment about 15% to about 25%, by weight of the total composition.

In some embodiments, the oral care compositions also comprise at least one humectant. Any orally acceptable humectant can be used, including without limitation, polyhydric alcohols such as glycerin, sorbitol (optionally as a 70 wt. % solution in water), propylene glycol, xylitol or low molecular weight polyethylene glycols (PEGs) and mixtures thereof. Most humectants also function as sweeteners. In some embodiments, compositions comprise 15% to 70% or 30% to 65% by weight humectant. Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein. One or more humectants are optionally present in a total amount of from about 1 wt. % to about 70 wt. %, for example, from about 1 wt. % to about 50 wt. %, from about 2 wt. % to about 25 wt. %, or from about 5 wt. % to about 15 wt. %, by total weight of the composition. In some embodiments, humectants, such as glycerin are present in an amount that is at least 20%>, e.g., 20-40%, e.g., 25-35%.

Mouth-feel agents include materials imparting a desirable texture or other feeling during use of the composition. In some embodiments, the oral care compositions comprise at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly i-carrageenan (iota-carrageenan), cellulosic polymers such as hydroxyethyl cellulose, and water-soluble salts of cellulose ethers (e.g., sodium carboxymethyl cellulose and sodium carboxymethyl hydroxyethyl cellulose), carboxymethylcellulose (CMC) and salts thereof, e.g., CMC sodium, natural gums such as karaya, xanthan, gum arabic and tragacanthin, colloidal magnesium aluminum silicate, colloidal silica, starch, polyvinyl pyrrolidone, hydroxyethyl propyl cellulose, hydroxybutyl methyl cellulose, hydroxypropyl methyl cellulose, and hydroxyethyl cellulose and amorphous silicas, and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol© series. Particularly preferred Carbopols include Carbopol 934, 940, 941, 956, 974P, and mixtures thereof. Silica thickeners such as DT 267 (from PPG Industries) may also be used. One or more thickening agents are optionally present in a total amount of from about 0.01 wt. % to 15 wt. %, for example from about 0.1 wt. % to about 10 wt. %, or from about 0.2 wt. % to about 5 wt. %, by total weight of the composition. Some embodiments comprise sodium carboxymethyl cellulose (e.g., from 0.5 wt. %-1.5 wt. %). In certain embodiments, thickening agents in an amount of about 0.5% to about 5.0% by weight of the total composition are used. Thickeners may be present in an amount of from 1 wt % to 15 wt %, from 3 wt % to 10 wt %, 4 wt % to 9 wt %, from 5 wt % to 8 wt %, for example 5 wt %, 6 wt %, 7 wt %, or 8 wt %.

In some embodiments, the oral care compositions comprise at least one colorant. Colorants herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5% by total weight of the composition.

In some embodiments, the oral care composition further comprises an anti-calculus (tartar control) agent. Suitable anti-calculus agents include, but are not limited to: phosphates and polyphosphates, polyaminopropane sulfonic acid (AM PS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates. Soluble pyrophosphates are useful anticalculus agents.

The pyrophosphate salts can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The pyrophosphates also contribute to preservation of the compositions by lowering water activity, tetrasodium pyrophosphate (TSPP), tetrapotassium pyrophosphate, sodium tripolyphosphate, tetrapolyphosphate, sodium trimetaphosphate, sodium hexametaphosphate and mixtures thereof. The salts are useful in both their hydrated and unhydrated forms. An effective amount of pyrophosphate salt useful in the present composition is generally enough to provide least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %.

Other useful tartar control agents include polymers and co-polymers. In some embodiments, the oral care compositions include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene), having a molecular weight (M.W.) of about 30,000 to about 1,000,000, polyvinyl methyl ether/maleic anhydride (PVM/MA) copolymers such as GANTREZ® (e.g., GANTREZ® S-97 polymer). In some embodiments, the PVM/MA copolymer comprises a copolymer of methyl vinyl ether/maleic anhydride, wherein the anhydride is hydrolyzed following copolymerization to provide the corresponding acid. In some embodiments, PVM/MA copolymer has an average molecular weight (M.W.) of about 30,000 to about 1,000,000, e.g. about 300,000 to about 800,000, e.g., wherein the anionic polymer is about 1-5%, e.g., about 2%, of the weight of the composition. In some embodiments, the anti-calculus agent is present in the composition in an amount of from 0.2 weight % to 0.8 weight %; 0.3 weight % to 0.7 weight %; 0.4 weight % to 0.6 weight %; or about 0.5 weight %, based on the total weight of the composition. Copolymers are available for example as Gantrez AN 139(M.W. 500,000), AN 119 (M.W. 250,000) and S-97 Pharmaceutical Grade (M.W. 70,000), of GAF Chemicals Corporation. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethyl enically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000. Another useful class of polymeric agents includes polyamino acids, particularly those containing proportions of anionic surface-active amino acids such as aspartic acid, glutamic acid and phosphoserine.

In some embodiments, the oral care compositions comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

In some embodiments, the oral care compositions comprise a nutrient. Suitable nutrients include vitamins, minerals, amino acids, and mixtures thereof. Vitamins include Vitamins C and D, miamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), and mixtures thereof.

In some embodiments, the oral care compositions comprise at least one viscosity modifier, useful for example to help inhibit settling or separation of ingredients or to promote re-dispersibility upon agitation of a liquid composition. Any orally acceptable viscosity modifier can be used, including without limitation, mineral oil, petrolatum, clays and organo-modified clays, silicas and the like. One or more viscosity modifiers are optionally present in a total amount of from about 0.01 wt. % to about 10 wt. %, for example, from about 0.1 wt. % to about 5 wt. %, by total weight of the composition.

In some embodiments, the oral care compositions comprise antisensitivity agents, e.g., potassium salts such as potassium nitrate, potassium bicarbonate, potassium chloride, potassium citrate, and potassium oxalate; capsaicin; eugenol; strontium salts; chloride salts and combinations thereof. Such agents may be added in effective amounts, e.g., from about 1 wt. % to about 20 wt. % by weight based on the total weight of the composition, depending on the agent chosen.

In some embodiments, the oral care compositions comprise an antioxidant. Any orally acceptable antioxidant can be used, including butylated hydroxy anisole (BHA), butylated hydroxytoluene (BHT), vitamin A, carotenoids, co-enzyme Q10, PQQ, Vitamin A, Vitamin C, vitamin E, anethole-dithiothione, flavonoids, polyphenols, ascorbic acid, herbal antioxidants, chlorophyll, melatonin, and mixtures thereof.

In some embodiments, the oral care compositions comprise of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g about 0.01%, about 0.1%, about 0.5%, about 1%, about 2%, about 5%, about 6%, by weight of the composition. In some embodiments, compositions comprise tetrapotassium pyrophosphate, disodium hydrogenorthophoshpate, monosodium phosphate, and pentapotassium triphosphate. In some embodiments, compositions comprise tetrasodium pyrophosphate from 0.1-1.0 wt % (e.g., about 0.5 wt %).

In some embodiments, the oral care compositions comprise a source of calcium and phosphate selected from (i) calcium-glass complexes, e.g., calcium sodium phosphosilicates, and (ii) calcium-protein complexes, e.g., casein phosphopeptide-amorphous calcium phosphate. Any of the preceding compositions further comprising a soluble calcium salt, e.g., selected from calcium sulfate, calcium chloride, calcium nitrate, calcium acetate, calcium lactate, and combinations thereof.

In some embodiments, the oral care compositions comprise an additional ingredient selected from: benzyl alcohol, Methylisothizolinone (“MIT”), Sodium bicarbonate, sodium methyl cocoyl taurate (tauranol), lauryl alcohol, and polyphosphate. Some embodiments comprise benzyl alcohol that is present from 0.1-0.8 wt %., or 0.2 to 0.7 wt %, or from 0.3 to 0.6 wt %, or from 0.4 to 0.5 wt %, e.g. about 0.1 wt. %, about 0.2 wt. %, about 0.3 wt %, about 0.4 wt %, about 0.5 wt %, about 0.6 wt %, about 0.7 wt % or about 0.8 wt %.

In some embodiments, the oral care compositions comprise from 5%-40%, e.g., 10%-35%, e.g., about 15%, 25%, 30%, and 35% or more of water.

Methods are provided for protecting and maintaining tissue integrity of tissue that functions as a barrier in an individual, for improving tissue integrity of tissue that functions as a barrier in an individual, for repairing damage to tissue that functions as a barrier in an individual and for improving oral immunity provided by a tissue that functions as a barrier in an individual. The tissue may in some embodiments be oral tissue, such as oral epithelial barrier tissue and in some embodiments, gingival epithelial barrier tissue. The barrier may in some embodiments be a keratinocyte tight junction barrier of oral epithelium. The methods for protecting and maintaining tissue integrity of tissue that functions as a barrier in an individual and for improving tissue integrity of tissue that functions as a barrier in an individual may be performed on an individual susceptible to or at an elevated risk of damage or a reduction to the tissue integrity of tissue that functions as a barrier or who is susceptible to or at an elevated risk of diseases or conditions that can result in damage or a reduction to the tissue integrity of tissue that functions as a barrier. The methods for repairing damage to tissue that functions as a barrier in an individual may be performed on an individual who is in need thereof such as an individual who has been identified as having damage to tissue that functions as a barrier.

As discussed herein, individuals with periodontal disease such as, gingivitis or periodontitis, and/or cardiovascular disease, respiratory diseases, type 2 diabetes periodontal disease, preterm birth/low birth weight, or colorectal disease may be more susceptible to or at a higher risk of damage to or a reduction in the integrity of tissue that functions as a barrier. In some embodiments, including any described above, methods comprise the step of identifying the individual as having periodontal disease such as in some embodiments, gingivitis and in some embodiments, periodontitis. In some embodiments, including any described above, methods comprise the step of identifying the individual as having cardiovascular disease, respiratory diseases, type 2 diabetes periodontal disease, preterm birth/low birth weight, or colorectal disease. In some embodiments, including any described above, methods comprise the step of identifying the individual as having periodontal disease such as in some embodiments, gingivitis and in some embodiments, periodontitis, and the step of identifying the individual as having cardiovascular disease, respiratory diseases, type 2 diabetes periodontal disease, preterm birth/low birth weight, or colorectal disease.

As discussed herein, individuals experiencing chronic inflammation and periodontal tissue destruction, individuals experiencing a marked pro-inflammatory response which may include the presence of proinflammatory cytokines, such as TNF-α or individuals with Gram-negative anaerobic bacteria, such as P. gingivalis, initiating disease in their oral cavity may be more susceptible to or at a higher risk of damage to or a reduction in the integrity of tissue that functions as a barrier as are individuals experiencing an uncontrolled and exaggerated inflammatory response by resident and/or immune cells to the presence of these pathogens and their toxins that is inducing secretion of one or more inflammatory mediators and matrix metalloproteinases (MMPs) that modulate destruction of the tooth-supporting tissues or having Gram-negative anaerobic bacteria that produces proteolytic enzymes that cause degradation of cell-to-cell junctions and the disruption of the epithelial barrier, or have hemolytic activity that lyses erythrocytes and releases hemoglobin. In some embodiments, including any described above, the methods comprise the step of identifying the individual as having Gram-negative anaerobic bacteria, such as P. gingivalis, initiating disease in their oral cavity. In some embodiments, including any described above, the methods comprise the step of identifying the individual as experiencing an uncontrolled and exaggerated inflammatory response of resident and/or immune cells to the presence of these pathogens and their toxins that is inducing secretion of one or more inflammatory mediators and matrix metalloproteinases (MMPs) that modulate destruction of the tooth-supporting tissues. In some embodiments, including any described above, the methods comprise the step of identifying the individual as having Gram-negative anaerobic bacteria in such individual's oral cavity that produces proteolytic enzymes that cause degradation of cell-to-cell junctions and the disruption of the epithelial barrier, or have hemolytic activity that lyses erythrocytes and releases hemoglobin. In some embodiments, including any of those described above, the individual is identified as experiencing chronic inflammation and periodontal tissue destruction.

In some embodiments the tissue may be damaged by the presence of proinflammatory cytokines such as TNF-α, or by pathogenic bacteria such as pathogenic bacteria collagenase activity, hemolytic activity or by induction of the individual's proteases. In some embodiments, including any described above, the methods comprise the step of identifying the individual as experiencing a marked pro-inflammatory response which may include the presence of proinflammatory cytokines, such as TNF-α. In some embodiments the tissue may be damaged by the presence of proinflammatory cytokines such as TNF-α, or by pathogenic bacteria such as pathogenic bacteria collagenase activity, hemolytic activity or by induction of the individual's proteases.

Each of these methods comprise contacting the tissue that functions as a barrier with an effective amount of a composition comprising one or more sources of zinc ions, and optionally further comprising one or more amino acids selected from the group consisting of: arginine, alanine, asparagine, cysteine, glutamine, glycine, isoleucine, leucine, methionine, phenylalanine, proline, serine, threonine, tryptophan, tyrosine, valine, and amino acids which have an isoelectric point in range of pH 5.0 to 7.0. In some embodiments, the effective amount is sufficient to promote keratinocyte proliferation and keratinocyte migration. In some embodiments, the one or more sources of zinc ions is selected from the group consisting of: zinc chloride, zinc acetate, zinc gluconate, zinc sulphate, zinc fluoride, zinc citrate, zinc lactate, zinc oxide, zinc monoglycerolate, zinc tartrate, zinc pyrophosphate, zinc phosphate, zinc maleate, zinc malate, zinc carbonate, zinc ascorbate, zinc lysine hydrochloride and zinc chloride hydroxide monohydrate (TBZC). In some embodiments, such as any of those described above, the composition comprises zinc oxide in an amount of from 0.75 to 1.25 wt %, or zinc citrate in an amount of from 0.25 to 1.0 wt %, or a combination of zinc oxide in an amount of from 0.75 to 1.25 wt % and zinc citrate in an amount of from 0.25 to 1.0 wt %. In such composition that comprise zinc oxide and zinc citrate and the zinc oxide, the ratio of the amount of zinc oxide (by wt %) to zinc citrate (by wt %) may in some embodiments be 2:1, 2.5:1, 3:1, 3.5:1 or 4:1, based on the total weight of the composition. In some embodiments, such as any of those described above, the composition comprises arginine (in free form or salt form), such as L-arginine, in an amount of from 0.1% to 15%, based on the total weight of the composition, the weight of the amino acid being calculated as free form.

In some embodiments, such as any of those described above, the composition is an oral care composition, such as a for example a toothpaste, which in some embodiments may contain fluoride, such as for example stannous fluoride.

### EXAMPLES

### Example 1

Trans Epithelial Electric Resistance (TER) as a Measurement to Evaluate Effect of Oral Care Ingredients and Products on Oral Skin Barrier Function

In oral cavity, chemical, mechanical, and biological factors may disrupt the oral tissue integrity, leading to increase the tissue permeability, subsequently, increasing risk of tissue damage and infection. The in vitro TER method is used to evaluate the effect of oral care ingredient and products on oral soft tissue integrity.

Cell junctions are intercellular pathways that selectively permit the movement of molecules through cellular layers. In terms of biological function, cell junctions form a barrier between apical and basolateral cell surfaces and are crucial in the development and function of epithelial tissues. The ion permeability of cell junctions can be measured through a voltmeter in the form of Trans Epithelial Electric Resistance (TER). Measurements are quickly determined through an electrode with little to no disturbance to the cell layers. The resistance correlates to the integrity of the tissue, and confluent tissue layers with intact cell junctions lead to higher TER readings.

Experimental Details

Tissue Cultures

Epigingival tissues were purchased from Mattek Corporation for use in the experiment. These multilayered epithelial cells imitate human gingival tissue in-vivo and can withstand high concentrations of toothpaste solutions. Once the tissues arrived in the laboratory, individual tissues were reconstituted in 1 mL of Mattek provided GIN-100 maintenance medium overnight at 37° C. and 5% CO₂ before testing of samples. The medium was changed every day after treatment.

Evaluate the Ingredient Upon Sodium Lauryl Sulfate (SLS) Challenge

TER measurement experiment was performed comparing the neat active ingredients in solution and their effects on the physical integrity of Mattek Epigingival tissues. The solutions were prepared in water and resemble the concentrations of the active ingredients found in toothpaste. The solutions were then diluted with water to various concentrations. Treatments of the Mattek Epigingival tissues use the same procedure listed above and were done 3× over 3 days. On the third day after treatment, the treated tissues were exposed to 100 μL of 0.5% SLS solution for 2 minutes. After 2 minutes, the SLS solution was aspirated, and the tissues were rinsed 3× with 200 μL of PBS. TER measurements were taken before and after exposure to SLS.

Evaluate Test Toothpaste Compositions

The procedure for treating tissues is as followed: make 1:2 ratio by weight toothpaste to sterile H₂O. Add 100 μL of treatment to the apical layer of the tissue insert using a 100 μL pipette. Aspirate the treatment. Rinse with 200 μL of PBS 3×. TER measurements were taken before and after exposure to toothpaste slurry.

Example of Results

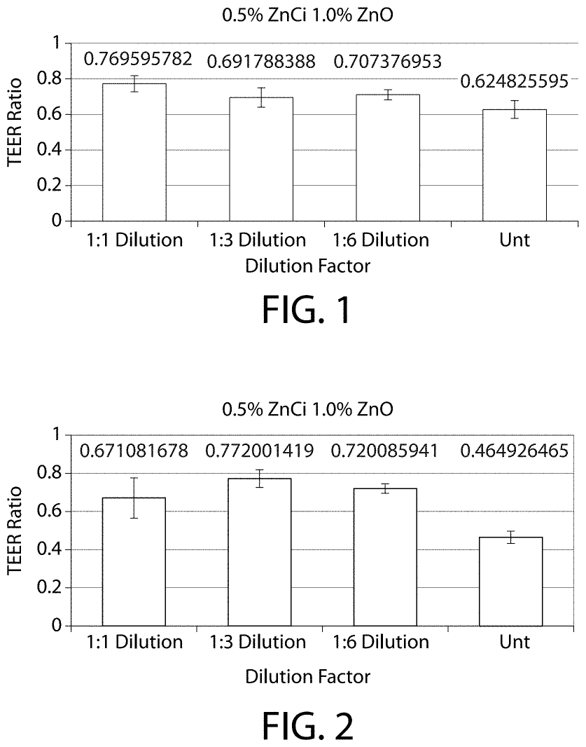

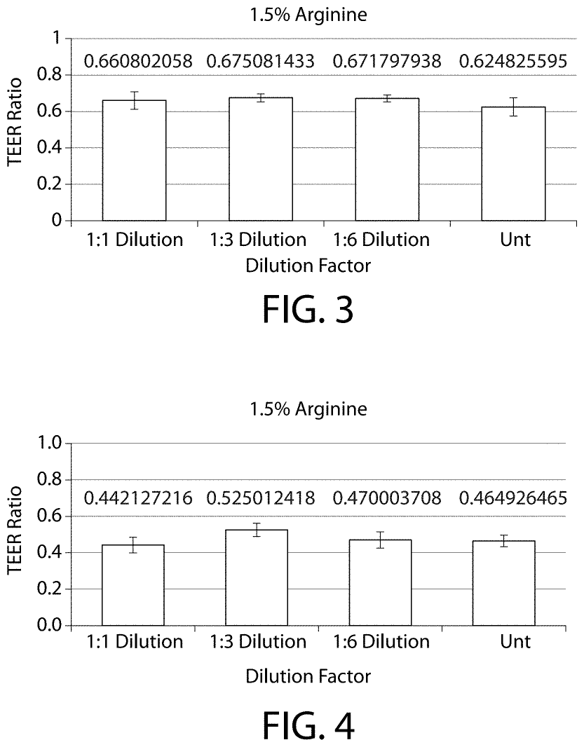

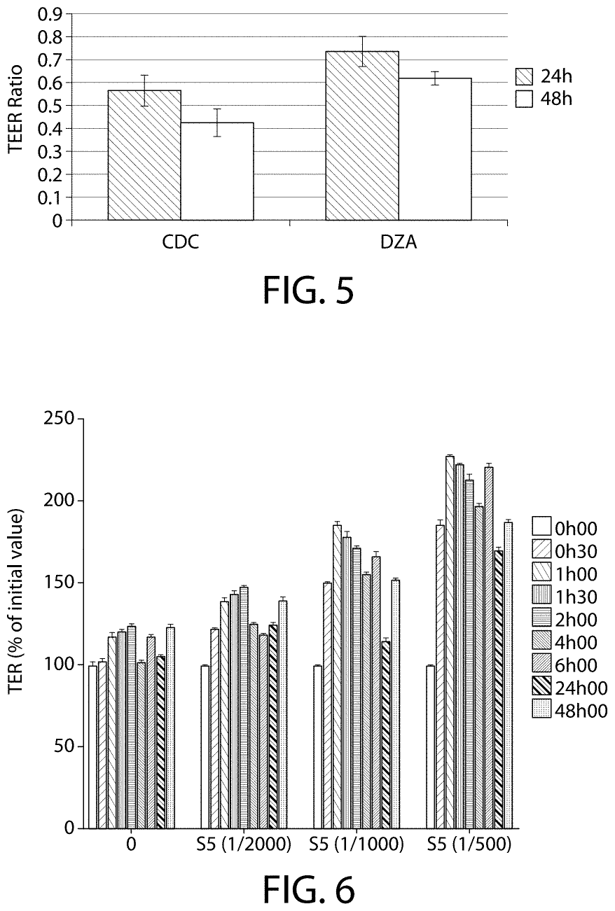

Treatment of the tissue with SLS induces in an increase in tissue permeability. A combination of 0.5% zinc citrate and 1% of zinc oxide prevents tissue permeability increasing induced by SLS (FIGS. 1 and 2). Arginine (1.5%) has minimal effect (FIGS. 3 and 4). The data in the graph in FIG. 1 show the TER ratio of zinc citrate-zinc oxide (Dual Zinc—0.5% Zinc citrate+1.0% Zinc oxide) after SLS exposure over before SLS exposure, comparing three different dilutions and a control that was untreated. The data in the graph in FIG. 2 show TER ratio of zinc citrate-zinc oxide (Dual Zinc 0.5% Zinc citrate+1.0% Zinc oxide) 60 hours after SLS exposure over immediately after SLS exposure, comparing three different dilutions and a control that was untreated. The data in the graph in FIG. 3 show TER ratio of arginine (1.5%) after SLS exposure over before SLS exposure, comparing three different dilutions and a control that was untreated. The data in the graph in FIG. 4 show TER ratio of arginine (1.5%) 60 hours after SLS exposure over immediately after SLS exposure, comparing three different dilutions and a control that was untreated. FIG. 5 shows data generated by comparing treatment of tissue with either a control fluoride toothpaste composition or a toothpaste formulation that comprises a combination of zinc oxide, zinc citrate and arginine. The data in the graph shows results from 24 hours and 48 hours after treatment. The data show the treatment with the toothpaste formulation that comprised a combination of zinc oxide, zinc citrate and arginine resulted in better tissue integrity compared to the treatment with the control fluoride toothpaste composition.

### Example 2

Methodology

The following assays were used in experiments to assess the effect of various test compositions on tissue integrity and to assess the effect of various test compositions on tissue integrity in the presence of a pathogenic bacteria or an inflammatory cytokine.

Human Gingival Keratinocyte Cultures

B11, an immortalized human gingival keratinocyte cell line was used to investigate the effect of samples on epithelial barrier integrity. Keratinocytes were cultivated in K-SFM supplemented with growth factors (50 μg/mL of bovine pituitary extract and 5 ng/mL of human epidermal growth factor) and 100 μg/mL of penicillin G-streptomycin at 37° C. in 5% CO₂ atmosphere.

Transepithelial Electrical Resistance

Tight junction integrity was assessed by determining the trans-epithelial electrical resistance (TER). B11 gingival keratinocytes were seeded onto Costar™ Transwell™ clear polyester membrane inserts (6.5-mm diameter; 0.4-μm pore size; Corning Co., Cambridge, Mass., USA) at 3×10⁵ cells per insert. The basolateral and apical compartments were filled with 0.6 mL and 0.1 mL of complete K-SFM, respectively. Following a 72-h incubation, the conditioned medium was replaced with antibiotic-free K-SFM, and the cells were incubated for a further 16 h. The TER values were measured using an Ohm/voltmeter (EVOM2; World Precision Instruments, Sarasota, Fla., USA) at 0, 4, 8, 24, 48, and 72 h. Resistance values were calculated in Ohms (Ω)/cm 2 by multiplying the resistance values by the filter surface area. Results were expressed as percentage of the basal control values measured at time 0 h (100% values) for each condition.

To investigate the effect of test composition samples on tight junction integrity, the medium in the apical compartment was supplemented with test composition.

Fluorescein Isothiocyanate-Conjugated Dextran Transport

The ability of test composition samples to enhance epithelial barrier integrity was assessed by monitoring the paracellular transport of FITC-conjugated 4.4-kDa dextran (FD-4; Sigma-Aldrich Canada Ltd.) across the keratinocyte layer. Briefly, B11 cells were cultured on Transwell filters, and FD-4 (1 mL-1 in culture medium) was added in the apical compartment in the presence of the test composition under investigation. Fluorescence in the basolateral compartment was measured at 0, 4, 8, 24, and 48 h using a Synergy 2 microplate reader (BioTek Instrument, Winooski, Vt., USA).

Immunofluorescent Staining of Zonula Occludens-1 and Occludin

Gingival keratinocytes treated as described above for 12 h were immunostained for zonula occludens-1 and occludin, two tight junction proteins. Briefly, the cells were fixed in 50 mM phosphate-buffered saline (pH 7.2) (PBS) containing 4% paraformaldhehyde for 20 min, permeabilized with 0.1% Triton X-100 for 10 min, and blocked in 3% non-fat milk in 20 mM Tris-HCl (pH 8), 150 mM NaCl, and 0.5% Tween 20 for 40 min. The cells were incubated with 2.5 μg/mL of either occludin antibody-Alexa Fluor® 488 conjugate or ZO-1 antibody-Alexa Fluor® 594 conjugate in blocking buffer overnight at 4° C. After washing with PBS, the cells were then treated with ProLong® Diamond antifade (Life Technologies). The slides were sealed with nail polish and were kept in the dark at 4° C. The localization of tight junction proteins in B11 cells was visualized using an Olympus FSX100 fluorescence microscope and FSX-BSW imaging software (Olympus, Tokyo, Japan). Immunofluorescent staining of tight junction proteins is a qualitative assay.

Test Solutions

In combinations of zinc oxide, zinc citrate and arginine used in assays, zinc oxide is present at 1%, zinc citrate is present at 0.5% and arginine is present at 1.5%. In assays testing zinc oxide, zinc oxide is present at 1%. In assays testing zinc citrate, zinc citrate is present at 2%. In assays, multiple dilutions of test compound are tested. Stock solutions are prepared in sterile distilled water. Thereafter, depending of the assay, dilutions are made in the appropriate diluent. Keratinocyte culture medium is used as diluent in assays that include keratinocytes. Bacterial culture medium is used as diluent in assays that use bacteria. Buffer is used in assays that test hemolytic and proteolytic activity.

### Example 3

The gingival epithelium, a stratified squamous tissue that acts as an interface between the external environment and the underlying connective tissue, plays an active role in maintaining oral health.

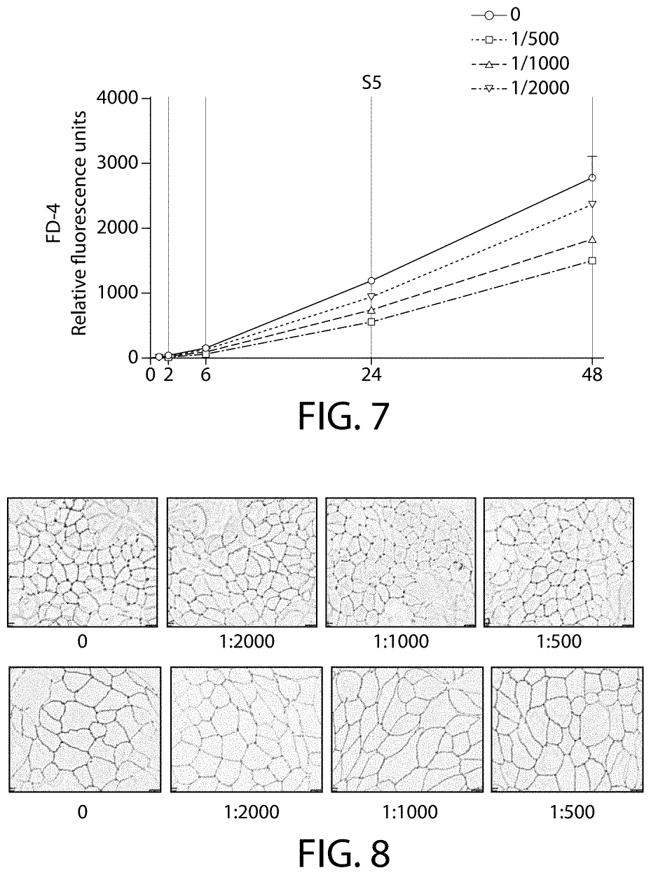

The capability of zinc oxide to reinforce the gingival epithelial barrier was evaluated using the methodology set out in Example 2. Data, which is shown in FIGS. 6-8 shows that ZnO (1%) improves oral tissue integrity. FIG. 6 shows data from the TER assay. The y-axis refers to TER measured as a percent of the initial value measured. The x-axis refers to the three dilutions (1/500, 1/1000 or 1/2000) of zinc oxide formulation tested plus the negative control (0). TER was measured at multiple time points for each dilution as indicated on bar graph. An increase in TER values indicates an epithelial barrier with a better integrity. The data show an increase in TER values with the increase in sample concentration. FIG. 7 shows data assessing paracellular permeability to FITC-dextran. The y-axis refers to FD-4 Relative fluorescence units measured. The x-axis refers to time point when measurements were taken over a 48-hour period (0, 2, 6, 24 and 48 h). The data in the graph is for the three dilutions (1/500, 1/1000 or 1/2000) of zinc oxide formulation tested plus the negative control (0). The control (0) showed the highest level of paracellular permeability to FITC-dextran. The paracellular permeability to FITC-dextran decreased with the increase in concentration of zinc oxide (1/500<1/1000<1/2000). The data demonstrate that zinc oxide improves oral tissue integrity. FIG. 8 shows photographs of immunofluorescence of tight junction proteins. The photos are of either cells treated with one of three dilutions (1/500, 1/1000 or 1/2000) of a 1% zinc oxide formulation or the negative control cells (0). The photos in the top row were stained for Occludin, which is green in original color. The photos in the bottom row were stained for Zonula Occludens-1, which is red in original color. Occludin stained green is shown in top row. Zonula Occludens-1 stained red and is shown in bottom row. The data show that zinc oxide enhances barrier integrity.

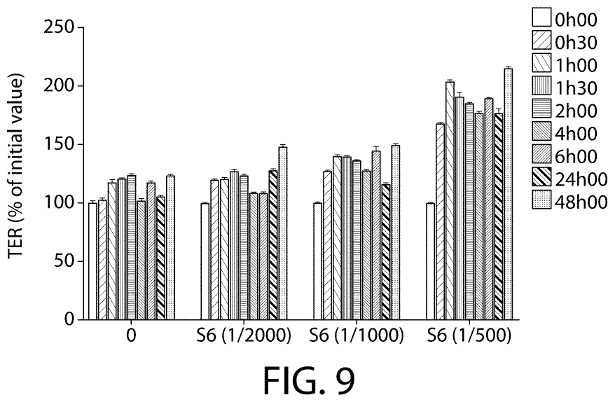

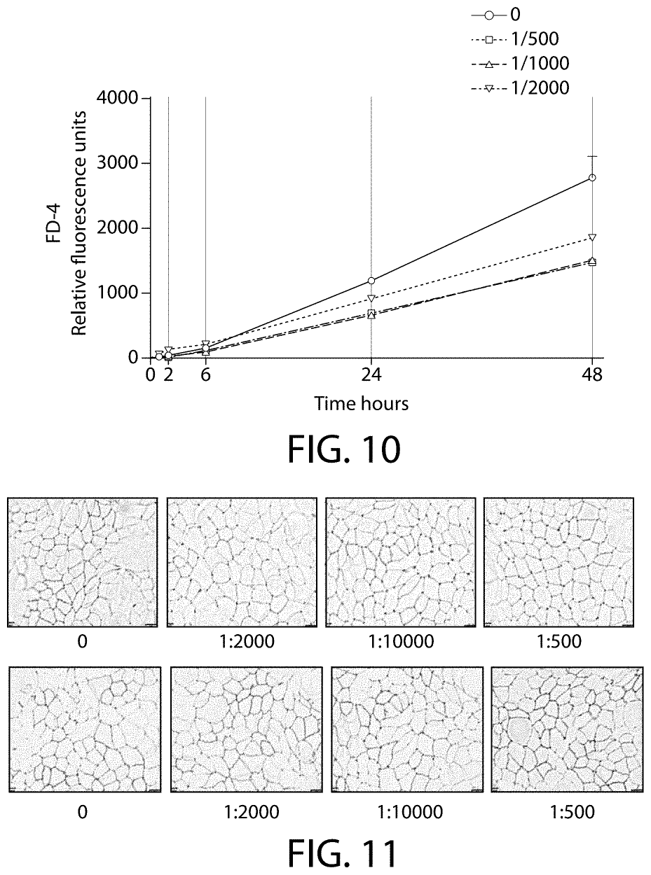

The capability of zinc citrate to reinforce the gingival epithelial barrier was evaluated using the methodology set out in Example 2. Data, which is shown in FIGS. 9-11 shows that zinc citrate improves oral tissue integrity. FIG. 9 shows data from the TER assay. The y-axis refers to TER measured as a percent of the initial value measured. The x-axis refers to the three dilutions (1/500, 1/1000 or 1/2000) of zinc citrate formulation (2%) tested plus the negative control (0). TER was measured at multiple time points for each dilution as indicated on bar graph. An increase in TER values indicates an epithelial barrier with a better integrity. The data show an increase in TER values with the increase in sample concentration, particular when used at the highest concentration. FIG. 10 shows data assessing paracellular permeability to FITC-dextran. The y-axis refers to FD-4 Relative fluorescence units measured. The x-axis refers to time points when measurements were taken over a 48-hour period (0, 2, 6, 24 and 48 h). The data in the graph is for the three dilutions (1/500, 1/1000 or 1/2000) of zinc citrate formulation (2%) tested plus the negative control (0). The control (0) showed the highest level of paracellular permeability to FITC-dextran. The paracellular permeability to FITC-dextran decreased with the increase in concentration of zinc citrate (1/500<1/1000<1/2000). The data demonstrate that zinc citrate improves oral tissue integrity. FIG. 11 shows photographs of immunofluorescence of tight junction proteins. The photos are of either cells treated with one of three dilutions (1/500, 1/1000 or 1/2000) of a zinc citrate formulation (2%) or the negative control cells (0). The photos in the top row were stained for Occludin, which is green in original color. The photos in the bottom row were stained for Zonula Occludens-1, which is red in original color. Occludin stained green is shown in top row. Zonula Occludens-1 stained red and is shown in bottom row. The data show that zinc citrate enhances barrier integrity.

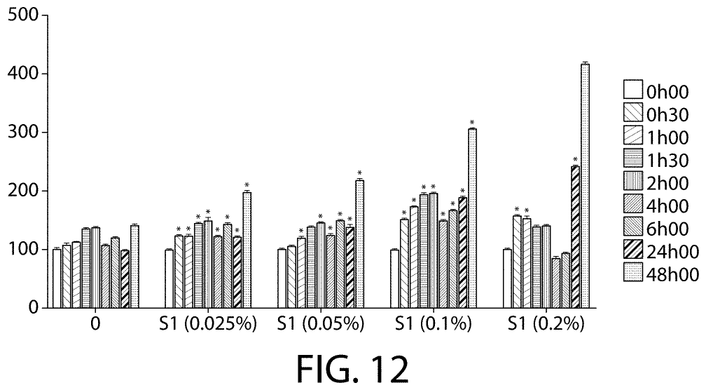

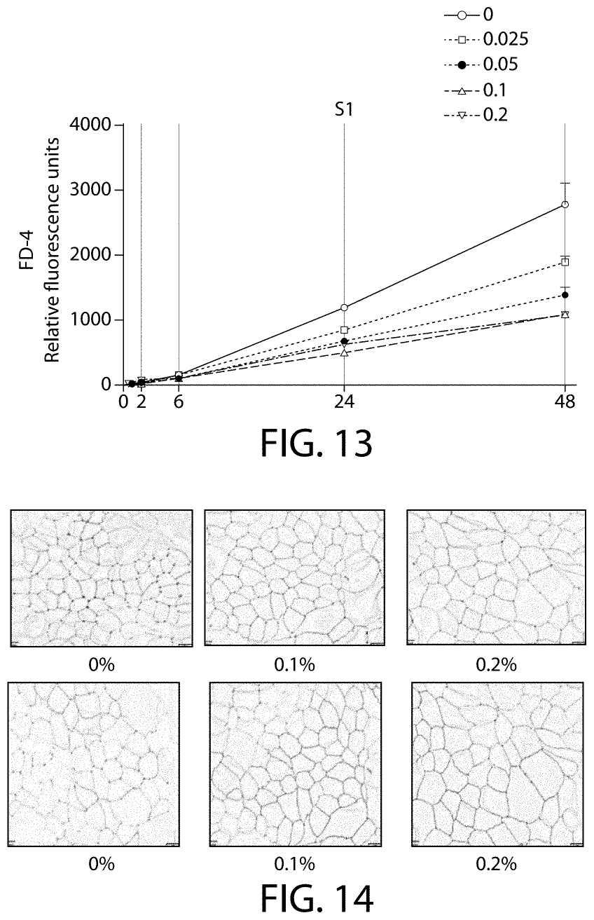

The capability of a formulation comprising zinc oxide+zinc citrate+arginine to reinforce the gingival epithelial barrier was evaluated using the methodology set out in Example 2. Data, which is shown in FIGS. 12-14 shows that zinc oxide+zinc citrate+arginine improves oral tissue integrity. FIG. 12 shows data from the TER assay. The y-axis refers to TER measured as a percent of the initial value measured. The x-axis refers to the four percentages (0.025%, 0.05%, 0.1%, 0.2%) of a formulation comprising zinc oxide (1%), zinc citrate (0.5%) and arginine (1.5%) tested plus the negative control (0). TER was measured at multiple time points for each dilution as indicated on bar graph. An increase in TER values indicates an epithelial barrier with a better integrity. The data show an increase in TER values with the increase in sample concentration, particular when used at the 0.1% concentration. FIG. 13 shows data assessing paracellular permeability to FITC-dextran. The y-axis refers to FD-4 Relative fluorescence units measured. The x-axis refers to time points when measurements were taken over a 48-hour period (0, 2, 6, 24 and 48 h). The data in the graph is for the four percentages (0.025%, 0.05%, 0.1%, 0.2%) of a formulation comprising zinc oxide (1%), zinc citrate (0.5%) and arginine (1.5%) tested plus the negative control (0). The control (0) showed the highest level of paracellular permeability to FITC-dextran. The paracellular permeability to FITC-dextran decreased with the increase in concentration of zinc oxide+zinc citrate+arginine (0.2%<0.1%<0.05%<0.025%). The data demonstrate that zinc oxide+zinc citrate+arginine improves oral tissue integrity. FIG. 14 shows photographs of immunofluorescence of tight junction proteins. The photos are of either cell treated with one of two percentages (0.1%, 0.2%) of a formulation comprising zinc oxide (1%), zinc citrate (0.5%) and arginine (1.5%) or the negative control cells (0). The photos in the top row were stained for Occludin, which is green in original color. The photos in the bottom row were stained for Zonula Occludens-1, which is red in original color. Occludin stained green is shown in top row. Zonula Occludens-1 stained red and is shown in bottom row. The data show that zinc oxide+zinc citrate+arginine enhances barrier integrity.

### Example 4

The gingival epithelium, a stratified squamous tissue that acts as an interface between the external environment and the underlying connective tissue, plays an active role in maintaining oral health. P. gingivalis is known to cause damage to the epithelial barrier. The capability of test compositions to protect oral tissue from perio-pathogenic bacteria caused tissue damage was assessed using the methodology set out in Example 2. P. gingivalis or P. gingivalis plus test composition were compared. P. gingivalis is added in the amount of 10⁴ MOI. P. gingivalis is suspended in keratinocyte culture medium at a concentration that will allow to obtain a final MOI of 10⁴ once the appropriate volume of medium is added to the keratinocyte culture. P. gingivalis and the test compounds are added simultaneously for all assays performed.

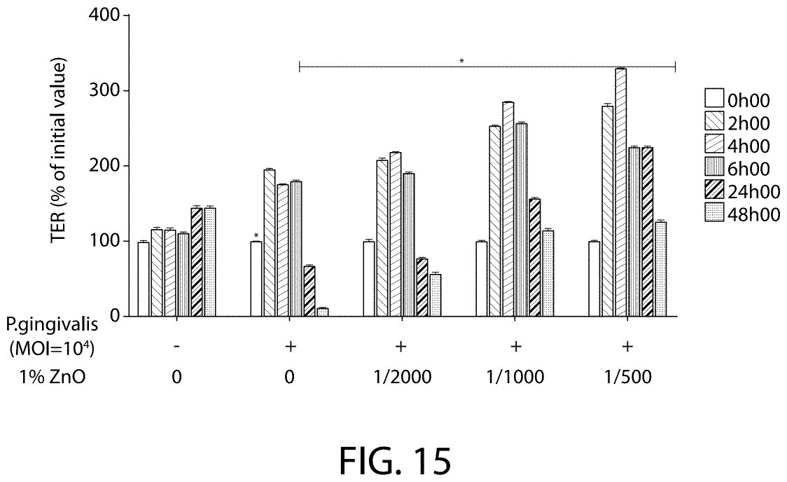

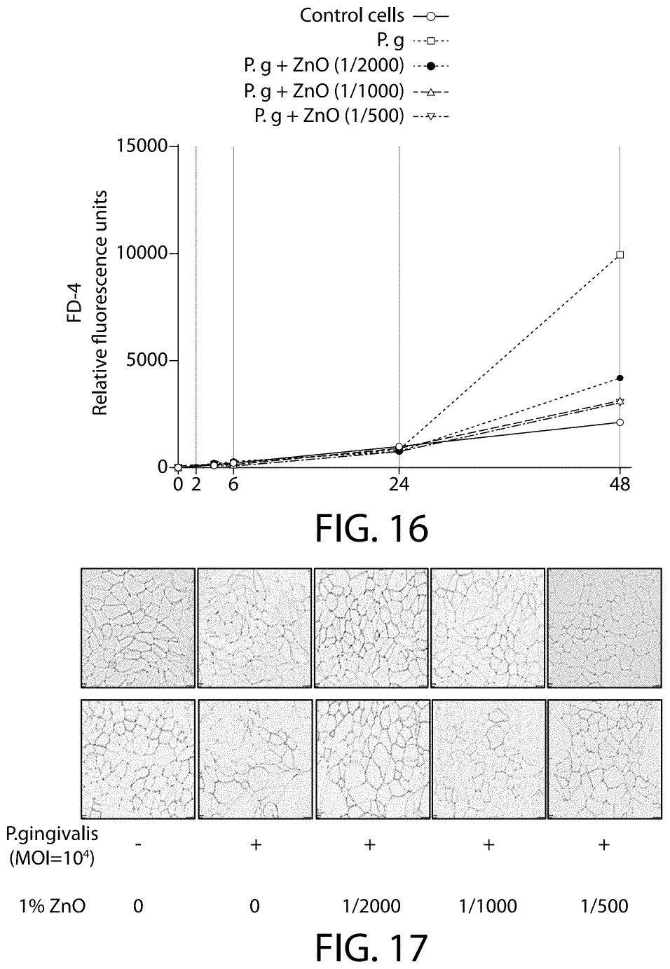

The capability of zinc oxide to protect oral tissue from perio-pathogenic bacteria caused tissue damage was tested. Data in FIGS. 15-17 show that zinc oxide protects tissue from perio-pathogenic bacteria induced damage. Zinc oxide was used at a concentration of 1%. MOI=10⁴ of the periopathogenic bacteria P. gingivalis was used. FIG. 15 shows data from the TER assay. The y-axis refers to TER measured as a percent of the initial value measured. The x-axis refers to a first control (no formulation, no P. gingivalis), a second control (P. gingivalis challenge, i.e. no formulation but with P. gingivalis) and the three dilutions (1/500, 1/1000 or 1/2000) of zinc oxide formulation (1%) in combination with P. gingivalis tested. TER was measured at multiple time points for each dilution as indicated on bar graph. An increase in TER values indicates an epithelial barrier with a better integrity. The data show an increase in TER values compared to the controls, with the increase in sample concentration, particular when used at the higher concentrations. FIG. 15 demonstrates that zinc oxide protects tissue integrity from damage by P. gingivalis. FIG. 16 shows data assessing paracellular permeability to FITC-dextran. The y-axis refers to FD-4 Relative fluorescence units measured. The x-axis refers to time points when measurements were taken over a 48-hour period (0, 2, 6, 24 and 48 h). The data in the graph is for a first control (no formulation, no P. gingivalis), a second control (P. gingivalis challenge, i.e. no formulation but with P. gingivalis) and the three dilutions (1/500, 1/1000 or 1/2000) of zinc oxide formulation (1%) in combination with P. gingivalis tested. The first control (no formulation, no P. gingivalis) showed the lowest level of paracellular permeability to FITC-dextran. The second control (P. gingivalis challenge, i.e. no formulation, but with P. gingivalis) showed the highest level of paracellular permeability to FITC-dextran. The paracellular permeability to FITC-dextran decreased with the increase in concentration of zinc oxide (1/500<1/1000<1/2000). The data demonstrate that zinc oxide protects oral tissue integrity from the deleterious effects caused by the presence of P. gingivalis. FIG. 17 shows photographs of immunofluorescence of tight junction proteins. The photos are of either cells treated with the first control (no formulation, no P. gingivalis), the second control (P. gingivalis challenge, i.e. no formulation but with P. gingivalis) and one of the three dilutions (1/500, 1/1000 or 1/2000) of zinc oxide formulation (1%) in combination with P. gingivalis. The photos in the top row were stained for Occludin, which is green in original color. The photos in the bottom row were stained for Zonula Occludens-1, which is red in original color. The data show that zinc oxide protects barrier integrity from the deleterious effects caused by the presence of P. gingivalis.

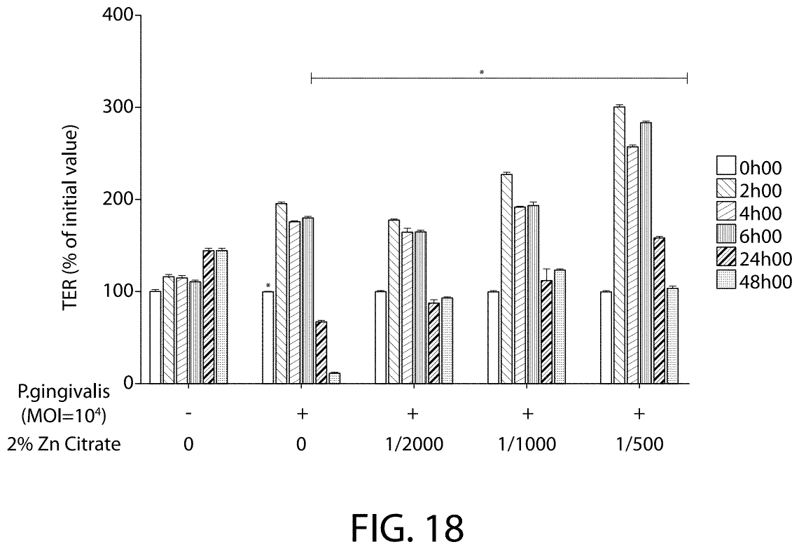

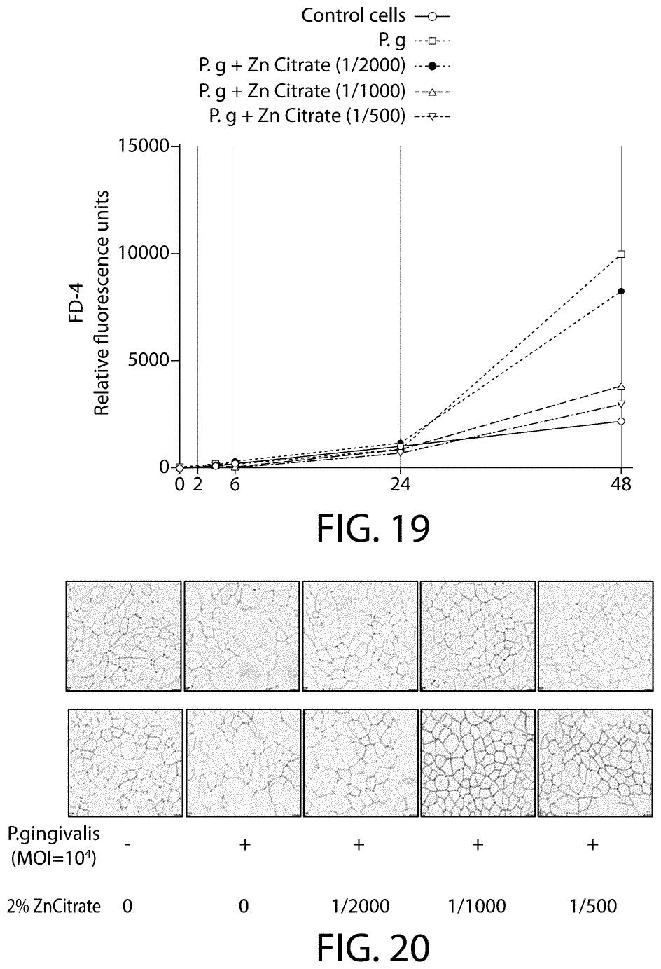

The capability of zinc citrate to protect oral tissue from perio-pathogenic bacteria caused tissue damage was tested. Data in FIGS. 18-20 show that zinc citrate protects tissue from perio-pathogenic bacteria induced damage. Zinc citrate was used at a concentration of 2%. MOI=10⁴ of the periopathogenic bacteria P. gingivalis was used. FIG. 18 shows data from the TER assay. The y-axis refers to TER measured as a percent of the initial value measured. The x-axis refers to a first control (no formulation, no P. gingivalis), a second control (P. gingivalis challenge, i.e. no formulation but with P. gingivalis) and the three dilutions (1/500, 1/1000 or 1/2000) of zinc citrate formulation (2%) in combination with P. gingivalis tested. TER was measured at multiple time points for each dilution as indicated on bar graph. An increase in TER values indicates an epithelial barrier with a better integrity. The data show an increase in TER values compared to the controls, with the increase in sample concentration, particularly when used at the highest concentration. FIG. 18 demonstrates that zinc citrate protects tissue integrity from damage by P. gingivalis. FIG. 19 shows data assessing paracellular permeability to FITC-dextran. The y-axis refers to FD-4 Relative fluorescence units measured. The x-axis refers to time points when measurements were taken over a 48-hour period (0, 2, 6, 24 and 48 h). The data in the graph is for a first control (no formulation, no P. gingivalis), a second control (P. gingivalis challenge, i.e. no formulation but with P. gingivalis) and the three dilutions (1/500, 1/1000 or 1/2000) of zinc citrate formulation (2%) in combination with P. gingivalis tested. The first control (no formulation, no P. gingivalis) showed the lowest level of paracellular permeability to FITC-dextran. The second control (P. gingivalis challenge, i.e. no formulation, but with P. gingivalis) showed the highest level of paracellular permeability to FITC-dextran. The paracellular permeability to FITC-dextran decreased with the increase in concentration of zinc citrate (1/500<1/1000<1/2000). The data demonstrate that zinc citrate protects oral tissue integrity from the deleterious effects caused by the presence of P. gingivalis. FIG. 20 shows photographs of immunofluorescence of tight junction proteins. The photos are of either cells treated with the first control (no formulation, no P. gingivalis), the second control (P. gingivalis challenge, i.e. no formulation but with P. gingivalis) and one of the three dilutions (1/500, 1/1000 or 1/2000) of zinc citrate formulation (2%) in combination with P. gingivalis. The photos in the top row were stained for Occludin, which is green in original color. The photos in the bottom row were stained for Zonula Occludens-1, which is red in original color. The data show that zinc citrate protects barrier integrity from the deleterious effects caused by the presence of P. gingivalis.

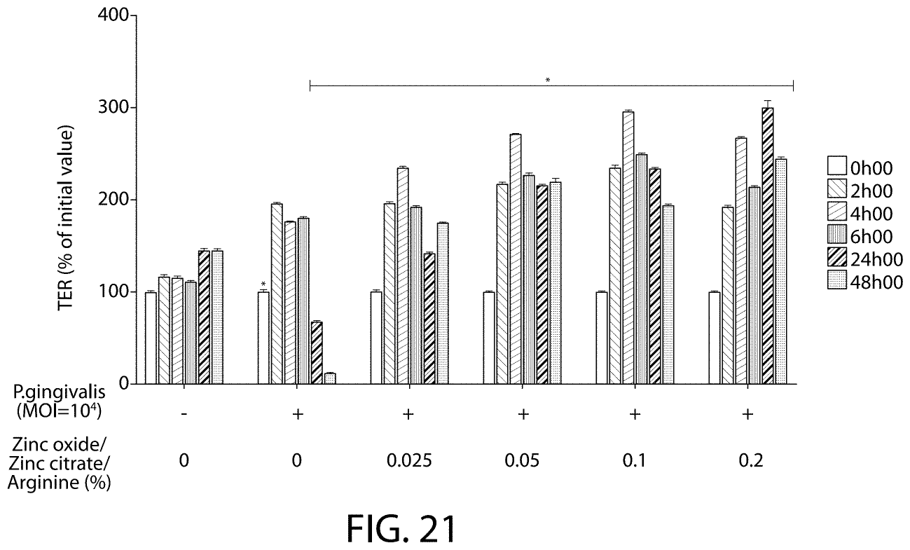

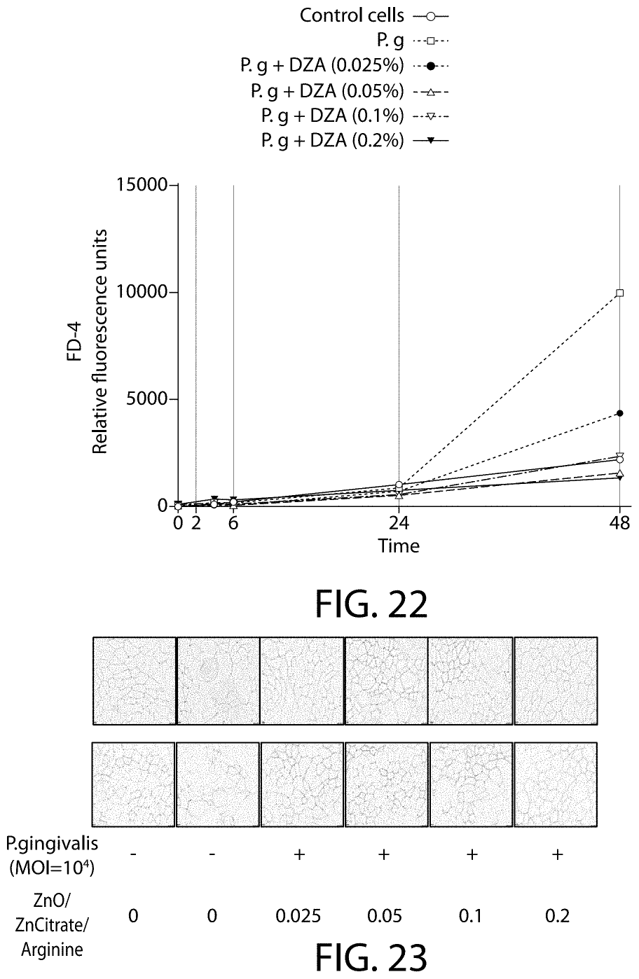

A composition comprising zinc oxide, zinc citrate and arginine was shown to attenuate the pathogenic properties of P. gingivalis. The capability of the combination of zinc oxide, zinc citrate and arginine to protect oral tissue from damage caused by periopathogenic bacteria was tested. Data in FIGS. 21-23 show that combination of zinc oxide, zinc citrate and arginine protects tissue from perio-pathogenic bacteria induced damage. MOI=10⁴ of the periopathogenic bacteria P. gingivalis was used. FIG. 18 shows data from the TER assay. The y-axis refers to TER measured as a percent of the initial value measured. The x-axis refers to a first control (no formulation, no P. gingivalis), a second control (P. gingivalis challenge, i.e. no formulation but with P. gingivalis) and four different percentages (0.025%, 0.05%, 0.1% and 0.2%) of the combined zinc oxide, zinc citrate and arginine formulation in combination with P. gingivalis tested. TER was measured at multiple time points for each dilution as indicated on bar graph. An increase in TER values indicates an epithelial barrier with a better integrity. The data show an increase in TER values compared to the controls, with the increase in sample concentration, particularly when used at the higher concentration. FIG. 21 demonstrates that the combination of zinc oxide, zinc citrate and arginine protects tissue integrity from damage by P. gingivalis. FIG. 22 shows data assessing paracellular permeability to FITC-dextran. The y-axis refers to FD-4 Relative fluorescence units measured. The x-axis refers to time points when measurements were taken over a 48-hour period (0, 2, 6, 24 and 48 h). The data in the graph is for a first control (no formulation, no P. gingivalis), a second control (P. gingivalis challenge, i.e. no formulation but with P. gingivalis) and the four different percentages (0.5%, 0.05%, 0.01% and 0.2%) of the combined zinc oxide, zinc citrate and arginine formulation in combination with P. gingivalis tested. The second control (P. gingivalis challenge, i.e. no formulation, but with P. gingivalis) showed the highest level of paracellular permeability to FITC-dextran. Two samples showed a lower level of paracellular permeability to FITC-dextran compared to the first control, one sample showed a level of paracellular permeability to FITC-dextran comparable to that of the first control and one samples showed a higher level of paracellular permeability to FITC-dextran compared to that of the first control. The data demonstrate that the combined zinc oxide, zinc citrate and arginine formulation can protect oral tissue integrity from the deleterious effects caused by the presence of P. gingivalis. FIG. 23 shows photographs of immunofluorescence of tight junction proteins. The photos are of either cells treated with the first control (no formulation, no P. gingivalis), the second control (P. gingivalis challenge, i.e. no formulation but with P. gingivalis) and one of the four percentages (0.025%, 0.05%, 0.1% and 0.2%) of a combined zinc oxide, zinc citrate and arginine formulation in combination with P. gingivalis. The photos in the top row were stained for Occludin, which is green in original color. The photos in the bottom row were stained for Zonula Occludens-1, which is red in original color. The data show that the combined zinc oxide, zinc citrate and arginine formulation protects barrier integrity from the deleterious effects caused by the presence of P. gingivalis.

### Example 5

Oral soft tissue problems such as gum disease involve gram-negative anaerobic bacteria and host cell interactions. The presence of proinflammatory cytokines damage the integrity of the tissue barrier. Contacting tissue with a proinflammatory cytokine such as TNFα reduces tissue integrity. Moreover, proinflammatory cytokines induce immune and inflammatory responses with the progression of the periodontal infection. Uncontrolled secretion of cytokines can occur, leading to chronic inflammation and periodontal tissue destruction. Most damage seen in periodontitis is host mediated. Host response plays a big role in periodontal disease development.

The capability of test compositions to protect tissue from proinflammatory cytokine induced tissue damage was tested using the methodology set out in Example 2. Assays exposed cells to either TNFα only or TNFα+test composition. TNFα is known to cause tissue damage. A stock solution of recombinant TNF-α is prepared in sterile distilled water at 100 μg/ml. Thereafter, dilutions are made in keratinocyte culture medium. First dilution is 1/1000 to obtain 100 ng/ml. TNF-α and the test compounds are added simultaneously.

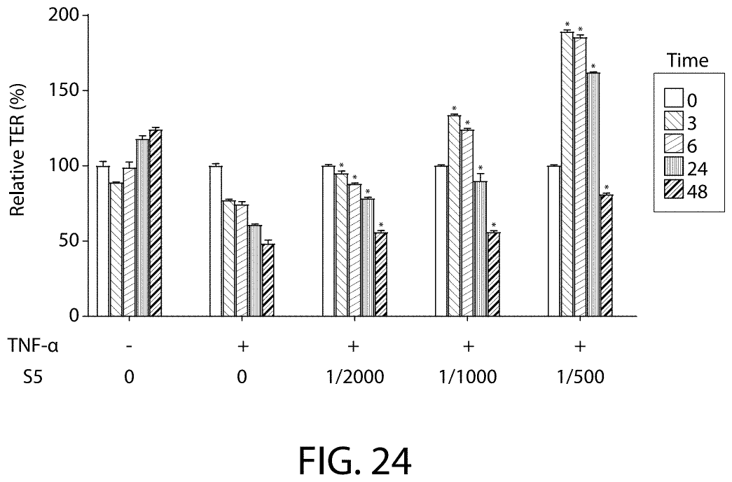

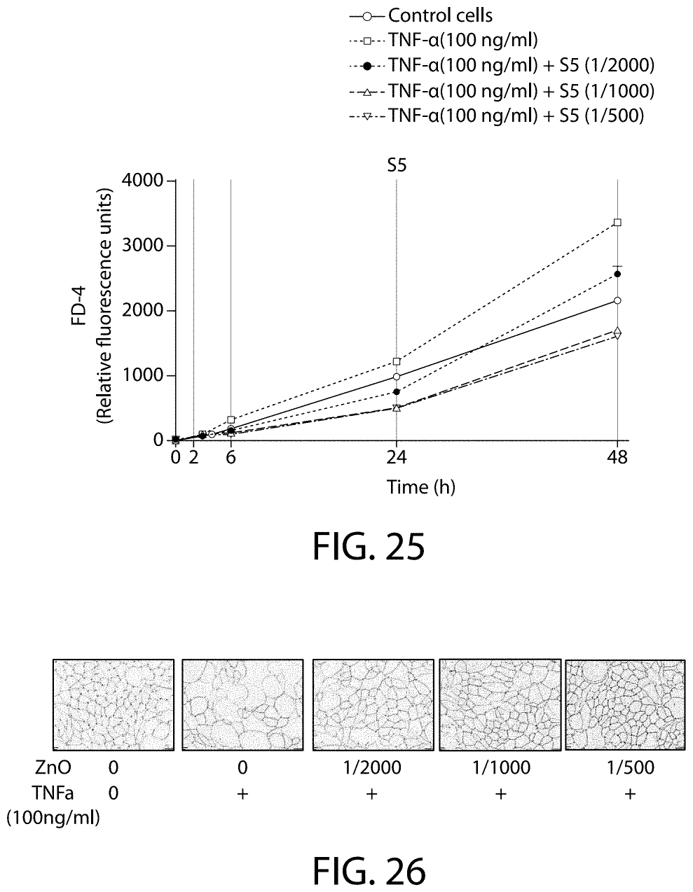

Zinc Oxide Protects Tissue from Proinflammatory Cytokine Induced Tissue Damage. The capability of zinc oxide to protect tissue from proinflammatory cytokine induced tissue damage was tested. Data in FIGS. 24-26 show that zinc oxide protects tissue from proinflammatory cytokine induced damage. FIG. 24 shows zinc oxide protects tissue from TNFα induced damage using the TER assay. The y-axis refers to TER measured as a percent of the initial value measured. The x-axis refers to a first control (no formulation, no TNFα), a second control (TNFα challenge, i.e. no formulation but with TNFα) and the three dilutions (1/500, 1/1000 or 1/2000) of zinc oxide formulation in combination with TNFα tested. TER was measured at multiple time points for each dilution as indicated on bar graph. An increase in TER values indicates an epithelial barrier with a better integrity. TER values for oral tissue decreased after proinflammatory cytokine TNFα treatment (second control). However, zinc oxide prevented tissue from the TNFα induced TER decrease. The data show an increase in TER values compared to the TNFα control, with the increase in sample concentration, particular when used at the higher concentrations. FIG. 24 demonstrates that zinc oxide protects tissue integrity from damage by TNFα. FIG. 25 shows data assessing paracellular permeability to FITC-dextran that demonstrates that zinc oxide prevents cells from TNFα-induced paracellular permeability to FITC-dextran. The y-axis refers to FD-4 Relative fluorescence units measured. The x-axis refers to time points when measurements were taken over a 48-hour period (0, 2, 6, 24 and 48 h). The data in the graph is for a first control (no formulation, no TNFα), a second control (TNFα challenge, i.e. no formulation but with TNFα) and the three dilutions (1/500, 1/1000 or 1/2000) of zinc oxide formulation in combination with TNFα tested. The second control (TNFα challenge, i.e. no formulation but with TNFα) showed the highest level of paracellular permeability to FITC-dextran. The two zinc oxide dilutions with the highest concentration of zinc oxide showed the lower level of paracellular permeability to FITC-dextran than the first control (no formulation, no TNFα). The two zinc oxide dilutions with the lower concentrations of zinc oxide showed the higher level of paracellular permeability to FITC-dextran compared to the first control (no formulation, no TNFα) but a lower level of paracellular permeability to FITC-dextran compared to the second control (TNFα challenge, i.e. no formulation but with TNFα). The data demonstrate that zinc oxide protects oral tissue integrity from the deleterious effects caused by the presence of TNFα. FIG. 26 shows that zinc oxide prevents TNFα induced cell junction disorganization. FIG. 26 shows photographs of immunofluorescence of tight junction proteins. The photos are of either cells treated with the first control (no formulation, no TNFα), the second control (TNFα challenge, i.e. no formulation but with TNFα) and one of the three dilutions (1/500, 1/1000 or 1/2000) of zinc oxide formulation in combination with TNFα. The photos were stained for Zonula Occludens-1, which is red in original color. The data show that zinc oxide protects barrier integrity from the deleterious effects caused by the presence of TNFα.

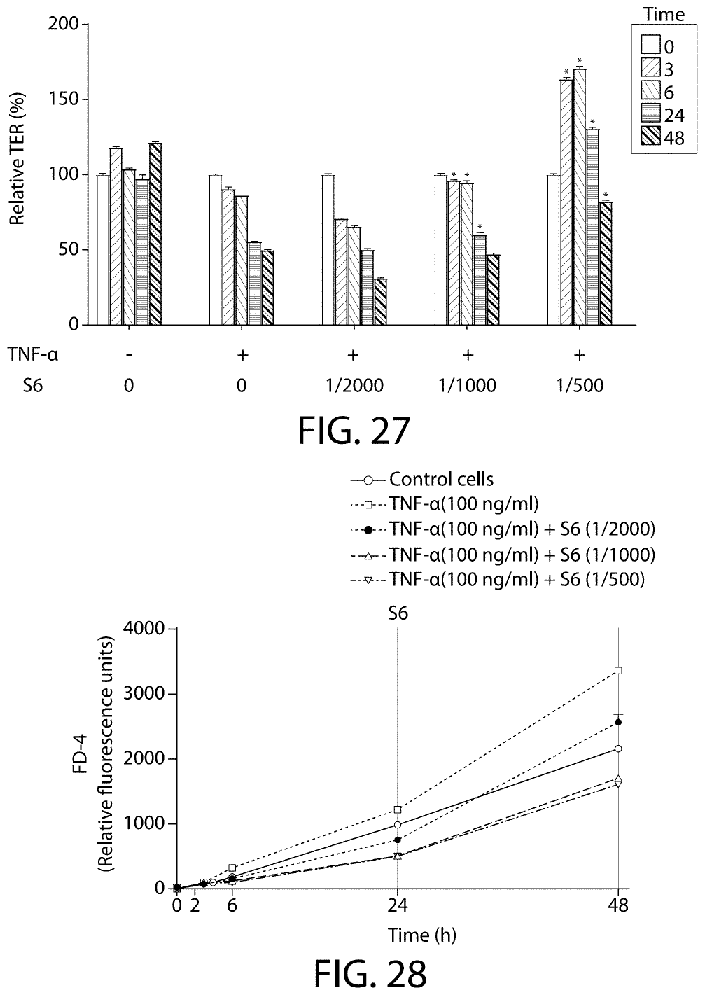

Zinc Citrate Protects Tissue from Proinflammatory Cytokine Induced Tissue Damage. The capability of zinc citrate to protect tissue from proinflammatory cytokine induced tissue damage was tested. Data, which is shown in FIGS. 27 and 28 shows that zinc citrate protects oral tissue from proinflammatory cytokine induced tissue damage. FIG. 27 shows zinc citrate protects tissue from TNFα induced damage using the TER assay. The y-axis refers to TER measured as a percent of the initial value measured. The x-axis refers to a first control (no formulation, no TNFα), a second control (TNFα challenge, i.e. no formulation but with TNFα) and the three dilutions (1/500, 1/1000 or 1/2000) of zinc citrate formulation in combination with TNFα tested. TER was measured at multiple time points for each dilution as indicated on bar graph. An increase in TER values indicates an epithelial barrier with a better integrity. TER values for oral tissue decreased after proinflammatory cytokine TNFα treatment (second control). However, zinc citrate can prevent tissue from the TNFα induced TER decrease. The data show that when zinc citrate is used at the higher concentrations, an increase in TER values compared to the TNFα control is observed. FIG. 27 demonstrates that zinc citrate protects tissue integrity from damage by TNFα. FIG. 28 shows data assessing paracellular permeability to FITC-dextran and demonstrates that zinc citrate prevents cells from TNFα-induced paracellular permeability to FITC-dextran. The y-axis refers to FD-4 Relative fluorescence units measured. The x-axis refers to time points when measurements were taken over a 48-hour period (0, 2, 6, 24 and 48 h). The data in the graph is for a first control (no formulation, no TNFα), a second control (TNFα challenge, i.e. no formulation but with TNFα) and the three dilutions (1/500, 1/1000 or 1/2000) of zinc citrate formulation in combination with TNFα tested. The second control (TNFα challenge, i.e. no formulation but with TNFα) showed the highest level of paracellular permeability to FITC-dextran. The two zinc citrate dilutions with the highest concentration of zinc citrate showed the lower level of paracellular permeability to FITC-dextran than the first control (no formulation, no TNFα). The two zinc citrate dilutions with the lower concentrations of zinc citrate showed the higher level of paracellular permeability to FITC-dextran compared to the first control (no formulation, no TNFα) but a lower level of paracellular permeability to FITC-dextran compared to the second control (TNFα challenge, i.e. no formulation but with TNFα). The data demonstrate that zinc citrate protects oral tissue integrity from the deleterious effects caused by the presence of TNFα.

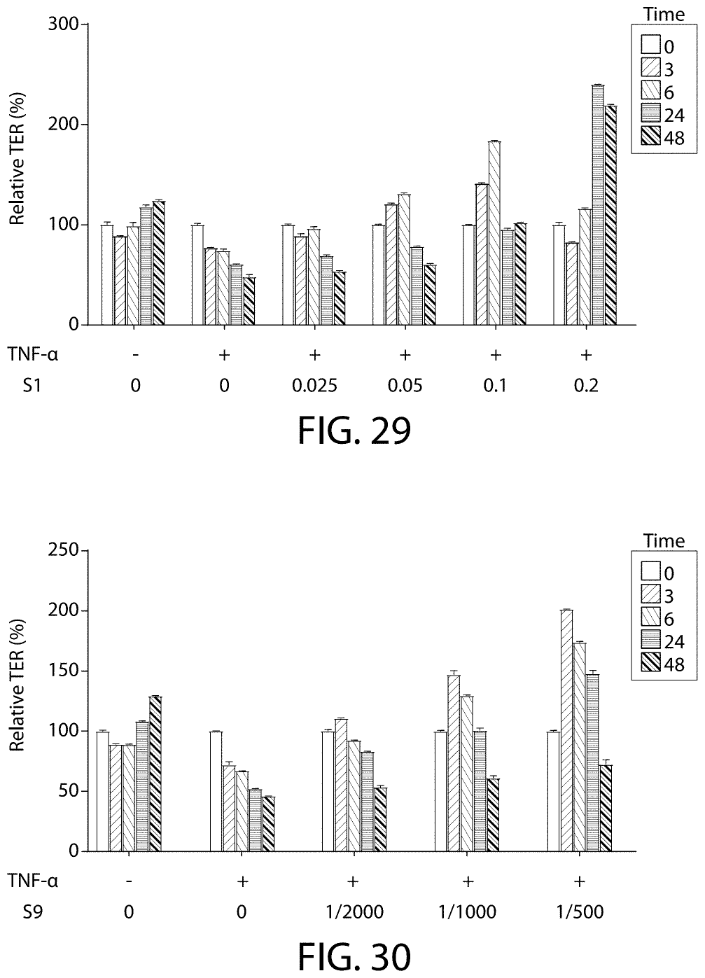

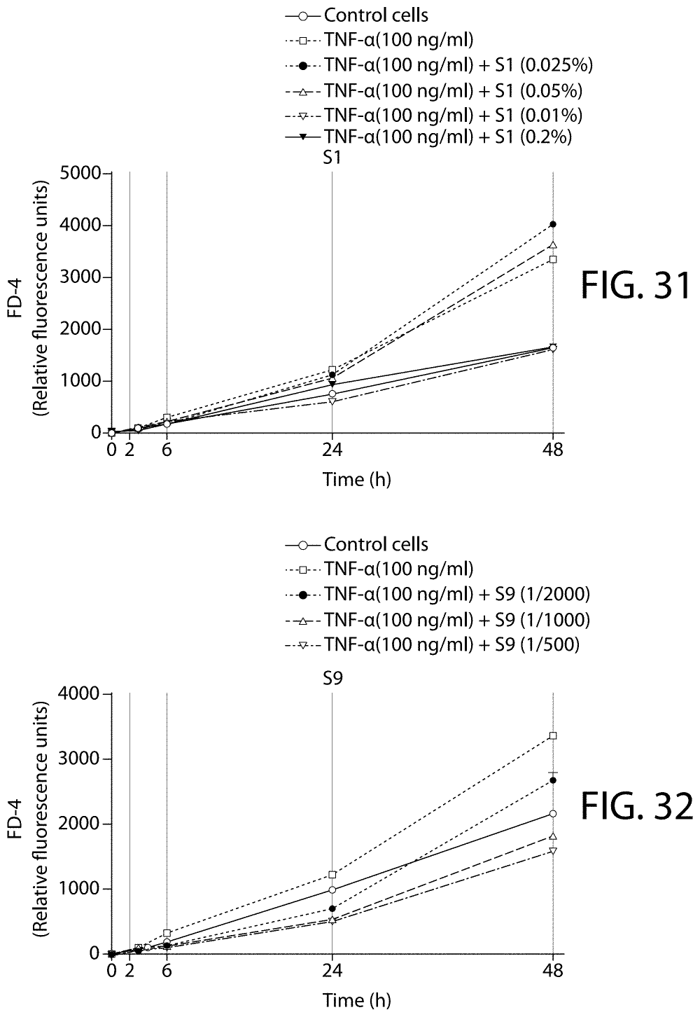

Combinations of Zinc Oxide, Zinc Citrate and Arginine Protect Tissue from Proinflammatory Cytokine Induced Tissue Damage. The capability of two different formulations that contain combinations of zinc oxide, zinc citrate and arginine to protect tissue from proinflammatory cytokine induced tissue damage was tested. Data, which is shown in FIGS. 29-34 shows that combinations of zinc oxide, zinc citrate and arginine protect oral tissue from proinflammatory cytokine induced tissue damage. FIG. 29 shows that a combination of zinc oxide, zinc citrate and arginine Zinc citrate (a mixture—S1) protects tissue from TNFα induced damage using the TER assay. FIG. 30 shows that a different combination of zinc oxide, zinc citrate and arginine (a toothpaste formulation—S9) protects tissue from TNFα induced damage using the TER assay. These data show that TER decreased after proinflammatory cytokine TNFα treatment. However, each combination of zinc oxide, zinc citrate and arginine prevented tissue from TNFα induced TER decreasing. In FIG. 29 and FIG. 30, the y-axis refers to TER measured as a percent of the initial value measured. In FIG. 29 the x-axis refers to a first control (no formulation, no TNFα), a second control (TNFα challenge, i.e. no formulation but with TNFα) and the four percentages (0.025%, 0.05%, 0.1% or 0.2%) of the zinc oxide, zinc citrate and arginine (a mixture—S1) in combination with TNFα tested. In FIG. 30, the x-axis refers to a first control (no formulation, no TNFα), a second control (TNFα challenge, i.e. no formulation but with TNFα) and the three dilutions (1/2000, 1/1000 and 1//500) of the zinc oxide, zinc citrate and arginine dentifrice formulation (S9) in combination with TNFα tested. For FIG. 29 and FIG. 30, TER was measured at multiple time points for each percentage and dilution, respectively, as indicated on bar graph. An increase in TER values indicates an epithelial barrier with a better integrity. TER values for oral tissue decreased after proinflammatory cytokine TNFα treatment (second control). However, zinc oxide, zinc citrate and arginine as a mixture S1 or dentifrice S9 prevented tissue from the TNFα induced TER decrease. The data show an increase in TER values compared to the TNFα control, with the increase in sample concentration, particular when used at the higher concentrations. FIG. 29 and FIG. 30 demonstrate that zinc oxide, zinc citrate and arginine protects tissue integrity from damage by TNFα. FIG. 31 and FIG. 32 show data assessing paracellular permeability to FITC-dextran that demonstrates that zinc oxide, zinc citrate and arginine prevents cells from TNFα-induced paracellular permeability to FITC-dextran. In FIG. 31 and FIG. 32, the y-axis refers to FD-4 Relative fluorescence units measured and the x-axis refers to time points when measurements were taken over a 48-hour period (0, 2, 6, 24 and 48 h). The data in the graph in FIG. 31 is for a first control (no formulation, no TNFα), a second control (TNFα challenge, i.e. no formulation but with TNFα) and the four percentages (0.025%, 0.05%, 0.1% or 0.2%) of the zinc oxide, zinc citrate and arginine (a mixture-S1) in combination with TNFα tested. The data in the graph in FIG. 32 is for a first control (no formulation, no TNFα), a second control (TNFα challenge, i.e. no formulation but with TNFα) and the three dilutions (1/2000, 1/1000 and 1//500) of the zinc oxide, zinc citrate and arginine dentifrice formulation (S9) in combination with TNFα tested. In FIG. 31, the two lowest percentage of the zinc oxide, zinc citrate and arginine (a mixture—S1) showed the higher level of paracellular permeability to FITC-dextran than the first control (no formulation, no TNFα) and the second control (TNFα challenge, i.e. no formulation but with TNFα) while the two higher percentage of the zinc oxide, zinc citrate and arginine (a mixture—S1) showed the level of paracellular permeability to FITC-dextran comparable to the first control (no formulation, no TNFα) and a lower level of paracellular permeability to FITC-dextran compared to the second control (TNFα challenge, i.e. no formulation but with TNFα). The data demonstrate that two higher percentage of the zinc oxide, zinc citrate and arginine (a mixture—S1) protects oral tissue integrity from the deleterious effects caused by the presence of TNFα. In FIG. 32, the lowest concentration of the zinc oxide, zinc citrate and arginine (a dentifrice—S9) showed the higher level of paracellular permeability to FITC-dextran than the first control (no formulation, no TNFα) and the lower level of paracellular permeability to FITC-dextran than the second control (TNFα challenge, i.e. no formulation but with TNFα) while the two higher concentrations of the zinc oxide, zinc citrate and arginine (a dentifrice—S9) showed the level of paracellular permeability to FITC-dextran lower than both the first control (no formulation, no TNFα) and the second control (TNFα challenge, i.e. no formulation but with TNFα). The data demonstrate that two higher percentage of the zinc oxide, zinc citrate and arginine (a mixture—S1) and each of the three dilutions of the zinc oxide, zinc citrate and arginine (a dentifrice—S9) protects oral tissue integrity from the deleterious effects caused by the presence of TNFα.

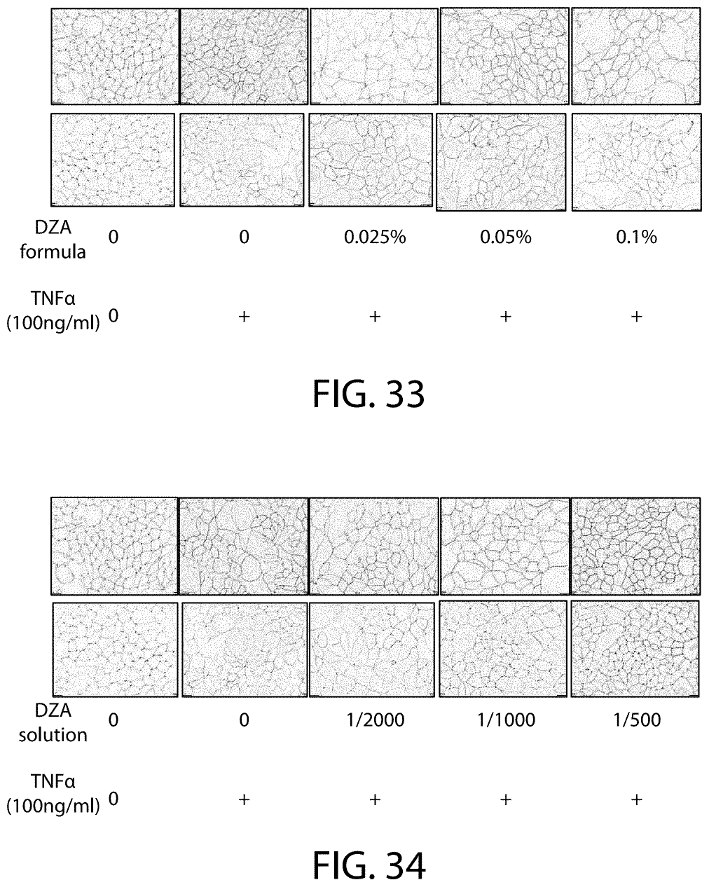

FIG. 33 and FIG. 34 show that zinc oxide, zinc citrate and arginine as a mixture—S1 and zinc oxide, zinc citrate and arginine as a dentifrice—S9 prevent TNFα induced cell junction disorganization. FIG. 33 and FIG. 34 show photographs of immunofluorescence of tight junction proteins. In FIG. 33 the photos are of either cells treated with the first control (no formulation, no TNFα), the second control (TNFα challenge, i.e. no formulation but with TNFα) and one of the three percentages (0.025%, 0.05%, 0.1%) of the zinc oxide, zinc citrate and arginine mixture—S1 formulation in combination with TNFα. In FIG. 34 the photos are of either cells treated with the first control (no formulation, no TNFα), the second control (TNFα challenge, i.e. no formulation but with TNFα) and one of the three dilutions (1/2000, 1/1000. 1/500) of the zinc oxide, zinc citrate and arginine dentifrice—S9 formulation in combination with TNFα. The photos in the top rows of FIG. 33 and FIG. 34 were stained for Occludin, which is green in original color. The photos in the bottom rows of FIG. 33 and FIG. 34 were stained for Zonula Occludens-1, which is red in original color. The data show that that zinc oxide, zinc citrate and arginine as a mixture—S1 and zinc oxide, zinc citrate and arginine as a dentifrice—S9 protect cell junction from TNFα induced cell junction damage.

### Example 6

Oral soft tissue problems such as gum disease involve gram-negative anaerobic bacteria and host cell interactions. Epithelial cells and fibroblasts are the predominant cells of periodontal tissues and serve as a first line of defense against periodontopathogens. They act as a mechanical barrier against bacterial invasion in addition to secreting different classes of inflammatory mediators and tissue-destructive enzymes in response to pathogen stimulation.

P. gingivalis produces collagenase, which negatively effects the integrity of the barrier formed by the tissue. The presence of collagenase results in an increase in tissue permeability. In addition, P. gingivalis collagenase activity reduces integrity of monolayer resulting in invasion of cell monolayer by P. gingivalis. Moreover, P. gingivalis can lyse cells.

To determine the effects of formulations tested on the proteinase activity of P. gingivalis, a 48-h culture was centrifuged at 10 000×g for 10 min. Assay mixtures containing equal volumes of P. gingivalis culture supernatant, the fluorescent substrate collagen DQ™ (100 μg/mL; Molecular Probes, Eugene, Oreg., USA), and the test compositions were prepared and incubated for 2 h at 37° C. The fluorescence corresponding to collagen degradation was monitored using a Synergy 2 microplate reader, with the excitation and emission wavelengths set at 495 nm and 525 nm, respectively. Test compounds or the fluorescent substrate alone were used as controls. For the proteolytic assay, the following ratio was used: 40% P. gingivalis supernatant+40% test compounds+20% DQ collagen. Incubation at 30° C. for up to 2 h.

Assays were performed to determine the extent formulations can inhibit and reduce invasion of cell monolayer by P. gingivalis.

A hemolytic assay is performed as follows. Fresh sheep red blood cells (Nutri-Bact, Terrebonne, QC, Canada) were harvested from heparinized whole blood by centrifugation (600×g for 5 min), washed three times in phosphate-buffered saline (PBS; pH 7.0), and suspended in PBS to a concentration of 2% (v/v). Equal volumes (1 mL) of red blood cells, P. gingivalis cells (OD660=1.0 in PBS), and two-fold serial dilutions of the Test composition were mixed together. PBS replaced the bacteria in the negative control. Following an incubation at 37° C. for 4 h, the mixtures were incubated at 4° C. for 1 h and were then centrifuged (10 000×g for 5 min) prior to recording the absorbance of the supernatants at 540 nm (A540). 1 ml P. gingivalis+1 ml of red blood cells+1 ml of test compounds used in the hemolysis assay are incubated at 37° C. for to 4 h. All constituents are added at the same time. For both assays, the dilution factor was considered for the test compounds in order to obtain the correct dilution to be tested.

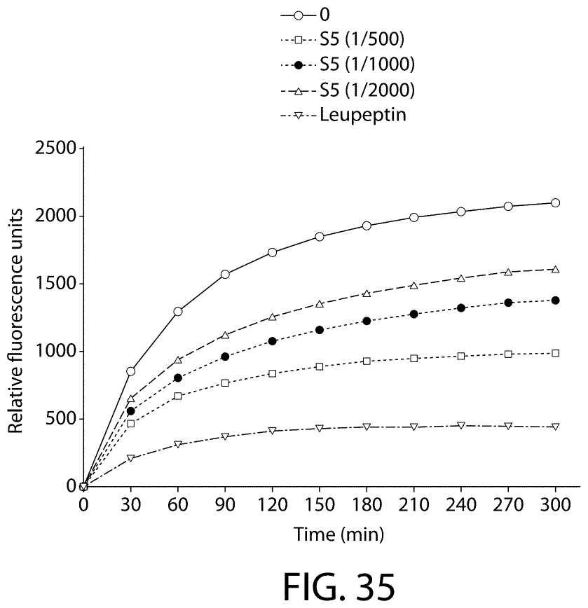

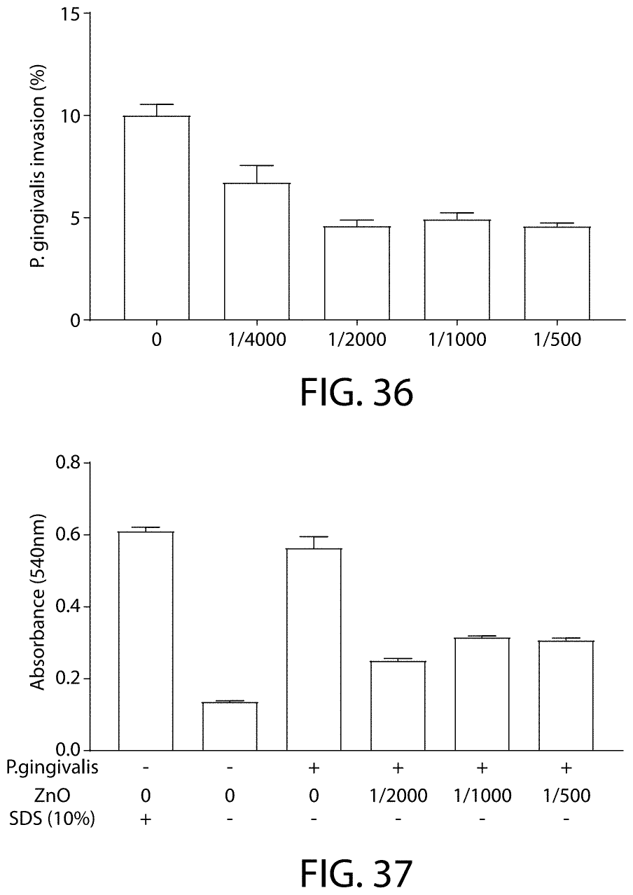

Zinc Oxide Attenuates P. gingivalis Pathogenic Properties. The capability of zinc oxide to inhibit P. gingivalis collagenase activity and to inhibit proteolytic enzymes that destroy gum tissue was evaluated. The capability of zinc oxide to inhibit translocation was evaluated by measuring the effect of zinc oxide on invasion of cell monolayer by P. gingivalis. In addition, assays to hemolytic properties of P. gingivalis and the capability of zinc oxide to inhibit hemolytic activity by P. gingivalis were evaluated. Data is shown in FIGS. 35-37. FIG. 35 shows that zinc oxide inhibits collagenase activity of P. gingivalis in a dose response manner. Experiments testing inhibition of P. gingivalis collagenase activity used a negative control (no zinc oxide formulation), a positive control (Leupeptin, a naturally occurring protease inhibitor that can inhibit P. gingivalis collagenase activity) and three dilutions (1/500, 1/1000 and 1/2000) of zinc oxide formulations. The y-axis refers to Relative fluorescence measured which corresponds to the amount of a labeled collagen substrate processed by collagenase from P. gingivalis. The x-axis refers to time point when measurements were taken over a 5-hour period. FIG. 35 shows that the negative control demonstrated the highest level of P. gingivalis collagenase activity—i.e. no inhibition— and positive control Leupeptin inhibited P. gingivalis collagenase activity. The data show that inhibition of P. gingivalis collagenase activity by the three dilutions (1/500, 1/1000 and 1/2000) of zinc oxide formulations was time- and dose-dependent. Higher concentrations of zinc oxide inhibited P. gingivalis collagenase activity at a higher level. FIG. 36 shows data demonstrating the effect of zinc oxide on invasion of an epithelial cell monolayer by P. gingivalis. The invasion of an epithelial cell monolayer by P. gingivalis was evaluated for a negative control and four dilutions (1/4000, 1/2000, 1/1000 and 1/500) of zinc oxide formulation. The y axis shows the level of P. gingivalis invasion of an epithelial cell monolayer. The x axis sets out the negative control and four dilutions. The results demonstrate that zinc oxide inhibits invasion of an epithelial cell monolayer. FIG. 37 shows data from experiments demonstrating that zinc oxide inhibits the hemolytic activity of P. gingivalis. A first control (no P. gingivalis, no zinc oxide, but with SDS), a second control (no P. gingivalis, no zinc oxide, no SDS), a third control (P. gingivalis but no zinc oxide and no SDS), and three dilutions (1/2000, 1/1000 and 1/500) of zinc oxide together with P. gingivalis but no SDS were tested to measure effect of zinc oxide on hemolytic activity of P. gingivalis on red blood cells from sheep. The data show that under non-denaturing conditions, P. gingivalis lysed red blood cells and that zinc oxide inhibited P. gingivalis hemolysis.

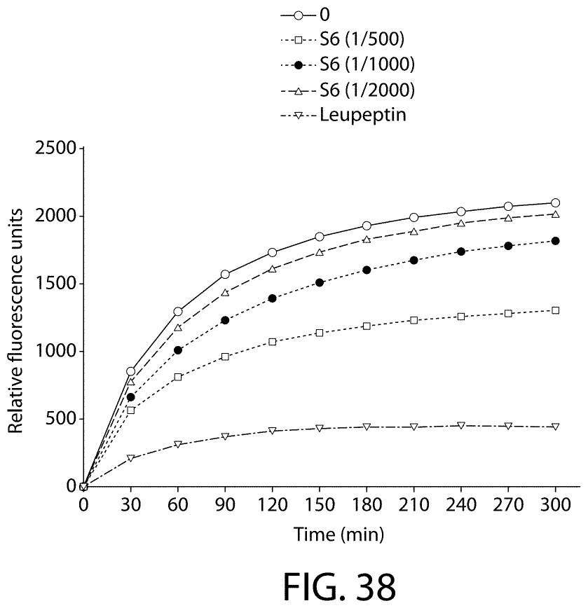

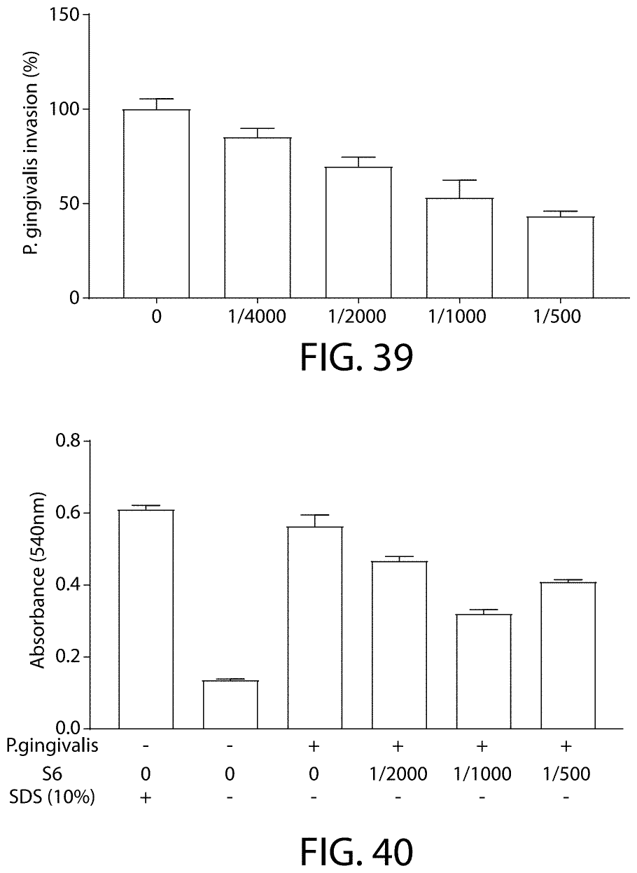

Zinc Citrate Inhibits Periopathogenic Properties of P. gingivalis. The capability of zinc citrate to inhibit P. gingivalis collagenase activity and to inhibit proteolytic enzymes that destroy gum tissue was evaluated. The capability of zinc citrate to inhibit translocation was evaluated by measuring the effect of zinc citrate on invasion of cell monolayer by P. gingivalis. In addition, assays to hemolytic properties of P. gingivalis and the capability of zinc citrate to inhibit hemolytic activity by P. gingivalis were evaluated. Data is shown in FIGS. 38-40. FIG. 38 shows that zinc citrate inhibits collagenase activity of P. gingivalis in a dose response manner. Experiments testing inhibition of P. gingivalis collagenase activity used a negative control (no zinc citrate formulation), a positive control (Leupeptin, a naturally occurring protease inhibitor that can inhibit P. gingivalis collagenase activity) and three dilutions (1/500, 1/1000 and 1/2000) of zinc citrate formulations. The y-axis refers to Relative fluorescence measured which corresponds to the amount of a labeled collagen substrate processed by collagenase from P. gingivalis. The x-axis refers to time point when measurements were taken over a 5-hour period. FIG. 38 shows that the negative control demonstrated the highest level of P. gingivalis collagenase activity—i.e. no inhibition— and positive control Leupeptin inhibited P. gingivalis collagenase activity. The data show that inhibition of P. gingivalis collagenase activity by the three dilutions (1/500, 1/1000 and 1/2000) of zinc citrate formulations was time- and dose-dependent. Higher concentrations of zinc citrate inhibited P. gingivalis collagenase activity at a higher level. FIG. 39 shows data demonstrating the effect of zinc citrate on invasion of an epithelial cell monolayer by P. gingivalis. The invasion of an epithelial cell monolayer by P. gingivalis was evaluated for a negative control and four dilutions (1/4000, 1/2000, 1/1000 and 1/500) of zinc citrate formulation. The y axis shows the level of P. gingivalis invasion of an epithelial cell monolayer. The x axis sets out the negative control and four dilutions. The results demonstrate that zinc oxide inhibits invasion of an epithelial cell monolayer. FIG. 40 shows data from experiments demonstrating that zinc citrate inhibits the hemolytic activity of P. gingivalis. A first control (no P. gingivalis, no zinc oxide, but with SDS), a second control (no P. gingivalis, no citrate oxide, no SDS), a third control (P. gingivalis but no zinc oxide and no SDS), and three dilutions (1/2000, 1/1000 and 1/500) of zinc citrate together with P. gingivalis but no SDS were tested to measure effect of zinc citrate on hemolytic activity of P. gingivalis on red blood cells from sheep. The data show that under non-denaturing conditions, P. gingivalis lysed red blood cells and that zinc citrate inhibited P. gingivalis hemolysis.

A combination of zinc oxide, zinc citrate and arginine attenuates periopathogenic properties of P. gingivalis.

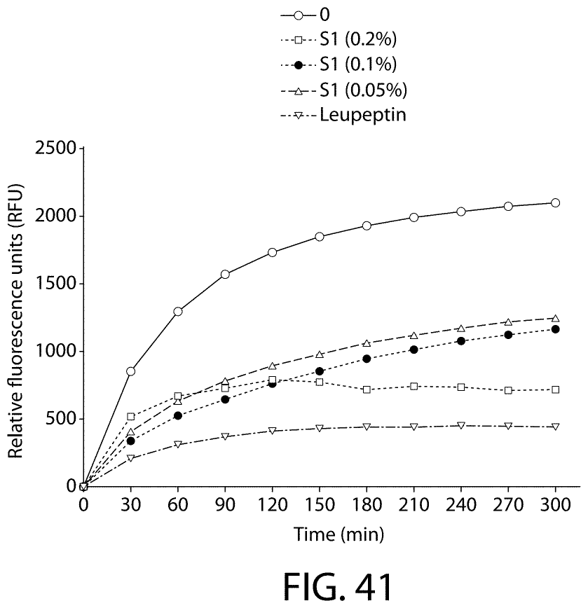

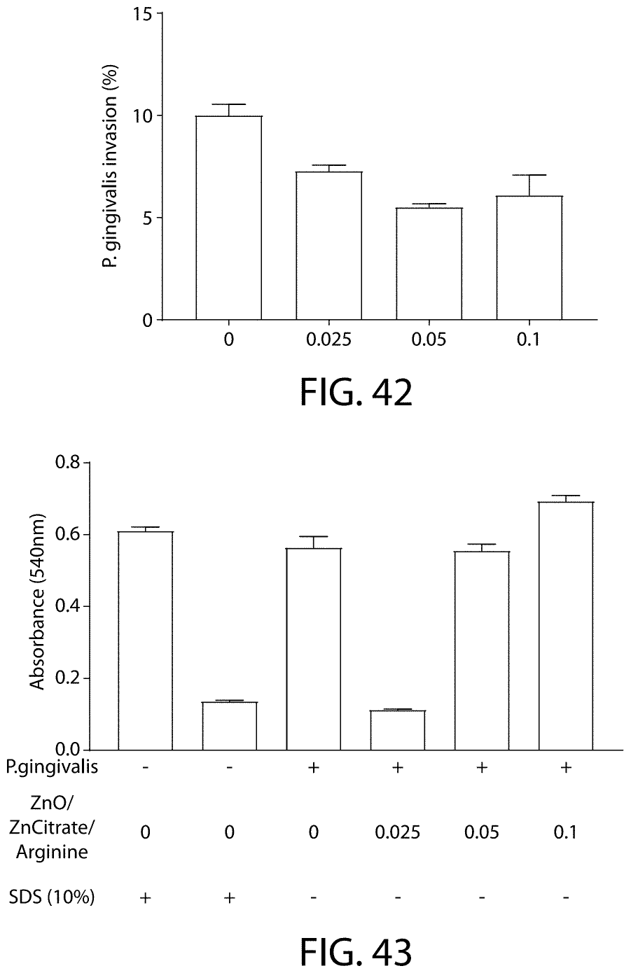

The capability of a combination of zinc oxide, zinc citrate and arginine to inhibit P. gingivalis collagenase activity and to inhibit proteolytic enzymes that destroy gum tissue was evaluated. The capability of the combination of zinc oxide, zinc citrate and arginine to inhibit translocation was evaluated by measuring the effect of zinc citrate on invasion of cell monolayer by P. gingivalis. In addition, assays to hemolytic properties of P. gingivalis and the capability of the combination of zinc oxide, zinc citrate and arginine to inhibit hemolytic activity by P. gingivalis were evaluated. Data is shown in FIGS. 41-43. FIG. 41 shows that the combination of zinc oxide, zinc citrate and arginine inhibits collagenase activity of P. gingivalis in a dose response manner. Experiments testing inhibition of P. gingivalis collagenase activity used a negative control (no zinc oxide, zinc citrate and arginine formulation), a positive control (Leupeptin, a naturally occurring protease inhibitor that can inhibit P. gingivalis collagenase activity) and samples with three different percentages (0.2%, 0.1%, 0.05%) of the combination of zinc oxide, zinc citrate and arginine of zinc citrate formulations. The y-axis refers to Relative fluorescence measured which corresponds to the amount of a labeled collagen substrate processed by collagenase from P. gingivalis. The x-axis refers to time point when measurements were taken over a 5-hour period. FIG. 41 shows that the negative control demonstrated the highest level of P. gingivalis collagenase activity—i.e. no inhibition— and positive control Leupeptin inhibited P. gingivalis collagenase activity. The data show that inhibition of P. gingivalis collagenase activity by the three samples with three different percentages (0.2%, 0.1%, 0.05%) of the combination of zinc oxide, zinc citrate and arginine of zinc citrate formulations was time- and dose-dependent. Higher concentrations of the combination of zinc oxide, zinc citrate and arginine inhibited P. gingivalis collagenase activity at a higher level. FIG. 42 shows data demonstrating the effect of the combination of zinc oxide, zinc citrate and arginine of zinc citrate formulations on invasion of an epithelial cell monolayer by P. gingivalis. The invasion of an epithelial cell monolayer by P. gingivalis was evaluated for a negative control and three samples with three different percentages (0.025%, 0.05%, 0.1%) of the combination of zinc oxide, zinc citrate and arginine of zinc citrate formulations. The y axis shows the level of P. gingivalis invasion of an epithelial cell monolayer. The x axis sets out the negative control and three different percentages (0.025%, 0.05%, 0.1%) of the combination of zinc oxide, zinc citrate and arginine formulations. The results demonstrate that combination of zinc oxide, zinc citrate and arginine inhibits invasion of an epithelial cell monolayer. FIG. 43 shows data from experiments demonstrating that combination of zinc oxide, zinc citrate and arginine inhibits the hemolytic activity of P. gingivalis. A first control (no P. gingivalis, no combination of zinc oxide, zinc citrate and arginine, but with SDS), a second control (no P. gingivalis, no combination of zinc oxide, zinc citrate and arginine, no SDS), a third control (P. gingivalis but no combination of zinc oxide, zinc citrate and arginine and no SDS), and three samples with three different percentages (0.025%, 0.05%, 0.1%) of the combination of zinc oxide, zinc citrate and arginine dilutions together with P. gingivalis but no SDS were tested to measure effect of the combination of zinc oxide, zinc citrate and arginine on hemolytic activity of P. gingivalis on red blood cells from sheep. The data show that under non-denaturing conditions, P. gingivalis lysed red blood cells and that the combination of zinc oxide, zinc citrate and arginine inhibited P. gingivalis hemolysis.

### Example 7

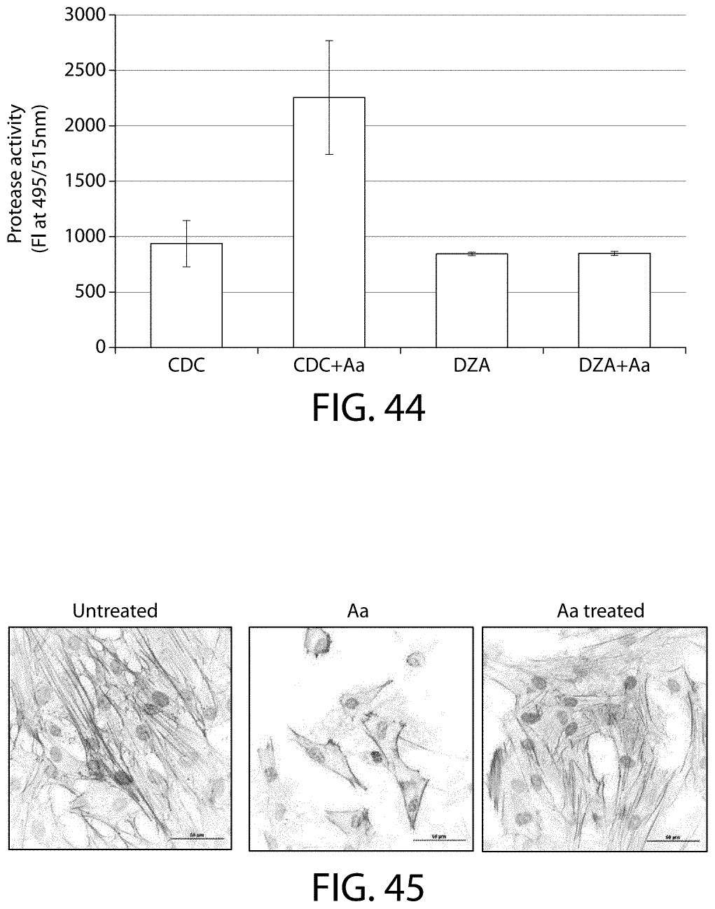

A combination of zinc oxide, zinc citrate and arginine protected oral tissue from damage caused by periopathogenic bacteria. Proteases such as MMPs are the critical virulent factor to degrade gum tissue in perioetiology by inhibiting Aggregatibacter actinomycetemcomitans (Aa) induced protease activity. When human cells are co-incubated with Aa, a periopathogenic bacteria, Gelatinase/collagenase (MMPs) activity significantly increased. When human cells are co-incubated with Aa in presence of a combination of zinc oxide, zinc citrate and arginine, the protease activity was reduced. Data is shown in FIG. 44. Images shown in FIG. 45 show the protective effect of the combination of zinc oxide, zinc citrate and arginine.

Procedure

Grow HEPM (human embryonic palatal mesenchyme cells) in 10 ml of DMEM containing 5% of FBS in a 48 well plate. Grow Aa in TSB. Wash HEPM cell once with PBS. Add DMEM with 0.5% FBS incubate for 1 hr. Make a combination of zinc oxide, zinc citrate and arginine), and a control slurry at 1:10 ratio of toothpaste:water. Read OD of Aa at 610 nm and resuspend Aa at OD 0.3 in DMEM (12 ml). Add as follows; Tube 1 to 3 ml of bacteria add 20 ul of zinc oxide, zinc citrate and arginine slurry. Tube 2 to 3 ml of DMEM add 20 ul of zinc oxide, zinc citrate and arginine slurry/Tube 3 to 3 ml of bacteria add 20 ul of control slurry. Tube 4 to 3 ml of DMEM. Add 20 ul of control slurry. Tube 5 to 3 ml of bacteria add 20 ul of water. Tube 6 to 3 ml of DMEM add 20 ul of water. Add 200 ul of each solution above to each designated well of 48 well plate with HEPM cells. 37c incubate for 60 mins in incubator. Aspirate media. Add 200 ul of each solution without Aa to each 48 well plate. 37c incubate for overnight in incubator. Transfer supernatant into a fresh plate for protease assay. Supernatant samples were used to conduct Protease assay (EnZChek gelatinase-collagenase assay kit cat#: E-12055, Molecular Probes at ThermoFisher) follow the manufacture's instruction. Previously, we had shown co incubate cells with Aa the proteolytic activity is high. But it is not in the same run. (IR 8083)

Procedure for Toothpaste Treatment—HEPM Cell

Culture Aa overnight. Grow HEPM on petri dishes overnight. Spin down Aa and resuspend in DMEM at about OD610=0.2. Make 1:10 diluted toothpaste slurry in water.

Wash HEPM cells with PBS once. Add 1 ml bacteria re-suspension to each HEPM cell petri dish except the one untreated control. To the untreated control, add 1 ml of DMEM media without bacteria. For treated with combination of zinc oxide, zinc citrate and arginine, add 5 ul of toothpaste slurry into each HEPM cell petri dish, incubate at 37° C. for 7-8 hs. Aspirate out media. Fix by add 1 ml of IC fixation (1:1 with PBS) at 4° C. overnight. Permeable with 0.25% triton-100 for 20 mins. Wash once with 1 ml of PBS. Block in 10% BSA at for 1 h at RT.

Add 200 ul PBS containing DAPI and Phalloidin 37° C. 30 mins to stain the nuclei and actin. Untreated control (no Aa)—cells are more flat and intact, actin (in green) well organized, extended to support the cell structure. Cells with Aa—cells are more shrunken, actin shortened. Cells with Aa in presence of combination of zinc oxide, zinc citrate and arginine—cells are more like intact.

FIG. 44 shows data from experiments testing inhibition of protease activity induced by Aggregatibacter actinomycetemcomitans (Aa) by a composition comprising a combination of zinc oxide-zinc citrate-arginine (a DZA composition). Human cell samples treated with a fluoride toothpaste composition, a fluoride toothpaste composition together with Aa, a composition comprising a DZA composition, and a DZA composition together with Aa and protease activity was measured. FIG. 45 shows photographic data from experiments described in Example 7 comparing the effect of a DZA composition on cells contacted with Aa. Nuclei and actin in cells were stained with DAPI and Phalloidin, respectively following treatment with Aa or Aa plus a DZA composition. Untreated control cells were also stained. In color photos, nuclei stain blue and actin stains green.

### Example 8

Porphyromonas gingivalis, a late colonizer of the periodontal biofilm, has been strongly associated with the chronic form of periodontitis. This Gram-negative bacterium produces a broad array of virulence factors that contribute to host tissue invasion and destruction. The aim was to investigate the antibacterial activity of a combination of zinc oxide, zinc citrate plus arginine in an aqueous solution and in a dentifrice against P. gingivalis and ex vivo periodontal plaque samples. The effects of each formulation on the pathogenic properties of P. gingivalis and the barrier function of an in vitro gingival epithelium model were also assessed. The zinc oxide, zinc citrate plus arginine in an aqueous solution and in a dentifrice each showed antibacterial activity against both P. gingivalis and ex vivo periodontal plaque samples. Moreover, each inhibited the hemolytic and proteolytic activities of P. gingivalis. The zinc oxide, zinc citrate plus arginine in an aqueous solution and in a dentifrice each enhanced the barrier function of an in vitro gingival epithelium model as determined by a time-dependent increase in transepithelial electrical resistance and paracellular permeability. This was associated with an increase in the immunolabelling of two important tight junction proteins: zonula occludens-1 and occludin. The deleterious effects of P. gingivalis on keratinocyte barrier function as well as the ability of the bacterium to translocate through a gingival epithelium model were abolished in the presence of zinc oxide, zinc citrate plus arginine in an aqueous solution as well as in the presence of zinc oxide, zinc citrate plus arginine in a dentifrice. In general, all the above beneficial properties were more marked when a fluoride dentifrice that included zinc oxide, zinc citrate plus arginine was used to compared to the beneficial effects observed when the zinc oxide, zinc citrate plus arginine in an aqueous solution was used. In conclusion, the zinc oxide, zinc citrate plus arginine formulation may offer benefits for patients affected by periodontal disease through its antibacterial activity as well as its ability to attenuate the pathogenic properties of P. gingivalis and promote epithelial barrier function.

Introduction

Various sites in the oral cavity are colonized by a wide range of microbial species, mainly bacteria, which interact with each other and with host cells, contributing to physiological and pathological conditions. Dental biofilm is initially formed by Gram-positive facultative anaerobic cocci and rods, including Streptococcus and Actinomyces species. As the dental biofilm matures, colonization shifts toward strictly anaerobic Gram-negative bacterial species that contribute to the subgingival biofilm that initiates periodontal disease (gingivitis, periodontitis). Disease progression and severity is modulated by a limited group of bacteria that challenge mucosal and immune cells, leading to the establishment of a chronic inflammatory condition. More specifically, periodontitis is characterized by irreversible and progressive destruction of the supporting tissues surrounding the teeth, including the alveolar bone.

Although periodontal disease is considered a multifactorial polymicrobial infection, Porphyromonas gingivalis is suspected to be one of the most important causative agents of the chronic form of this disease. This keystone bacterial species has been suggested to induce the transition from a symbiotic microbial community to a dysbiotic microbiota. P. gingivalis contributes to the pathogenesis of periodontitis through the expression of a wide range of virulence factors, including cysteine proteinases or gingipains that degrade tissue proteins, perturb host defense mechanisms, and modulate the host inflammatory response.

The oral epithelium creates a physical protective barrier between the underlying connective tissue and invasive periodontal pathogens and their toxic products in the oral environment, and thus plays an active role in the maintenance of periodontal health. The intercellular tight junction, which is composed of specialized transmembrane proteins that regulate transepithelial permeability, is the primary cellular determinant of epithelial barrier integrity and function. P. gingivalis has developed different strategies to compromise the structural and functional integrity of the oral epithelium. Experiments using specific gingipain inhibitors and gingipain-deficient mutants of P. gingivalis show that these proteolytic enzymes are involved in the degradation of cell-to-cell junctions and the disruption of the epithelial barrier. Once the integrity of the oral epithelium is disrupted, P. gingivalis, along with other periodontal pathogens, can reach deeper connective tissues and trigger a marked pro-inflammatory response that modulates tissue destruction. Bacteria and their toxins can also enter the bloodstream, migrate to extra-oral sites, and cause systemic complications.

A dentifrice formulation containing zinc (zinc oxide, zinc citrate) and arginine, known as Dual Zinc plus Arginine significantly decreased oral bacterial counts as well as plaque and gingivitis parameters compared to a regular fluoride dentifrice. The antibacterial activity of the Dual Zinc plus Arginine formulation as an aqueous solution and in a fluoride dentifrice was evaluated against P. gingivalis and ex vivo periodontal plaque samples. The effects of the Dual Zinc plus Arginine aqueous solution and dentifrice on the pathogenic properties of P. gingivalis and on the barrier function of an in vitro gingival epithelium model were also assessed.

Materials and Methods

Formulation

Zinc oxide and zinc citrate trihydrate were obtained from U.S. Zinc (Houston, Tex., USA) and Jost Chemical (St. Louis, Mo., USA), respectively. L-arginine was purchased from Ajinomoto (Tokyo, Japan). A mixture containing 0.96% zinc (zinc oxide, zinc citrate) and 1.5% arginine was freshly prepared in sterile distilled water and is referred to as the Dual Zinc plus Arginine aqueous solution. Unless indicated otherwise, the Dual Zinc plus Arginine aqueous solution was used at dilutions of 1/500, 1/1000, and 1/2000 (v/v). A dentifrice containing 0.96% zinc (zinc oxide, zinc citrate), 1.5% arginine, and 1450 ppm fluoride as sodium fluoride in a silica base toothpaste formula. A zinc and arginine-free control fluoride dentifrice was also tested. Unless indicated otherwise, the Dual Zinc plus Arginine dentifrice and control fluoride dentifrice were used at dilutions of 1/500, 1/1000, and 1/2000 (w/v). At the dilutions used, the amounts of zinc and arginine in the Dual Zinc plus Arginine aqueous solution and the Dual Zinc plus Arginine dentifrice were comparable. When the Dual Zinc plus Arginine aqueous solution and dentifrice were inoculated onto Todd-Hewitt agar plates (THA; Becton, Dickinson and Company, Sparks, MD, USA), no microbial contamination was observed (data not shown).

Bacteria and Growth Conditions

P. gingivalis ATCC 33277 was grown in an anaerobic chamber (80% N₂, 10% CO₂, 10% H₂) for 24 h at 37° C. in Todd-Hewitt broth (Becton, Dickinson and Company) supplemented with 0.001% (w/v) hemin and 0.0001% (w/v) vitamin K (THB-HK).

Growth Inhibitory Assay

P. gingivalis: An overnight bacterial culture was diluted in THB-HK to obtain an optical density at 660 nm)(OD₆₆₀) of 0.2. Aliquots (100 μL) were added to the wells of a flat-bottomed 96-well microplate containing two-fold serial dilutions in culture medium (100 μL) of the Dual Zinc plus Arginine aqueous solution or dentifrice (from dilution 1/15.625 to 1/2000). Wells with no bacteria or no compounds were used as controls. The microplate was incubated for 48 h at 37° C. in the anaerobic chamber prior to monitoring bacterial growth by recording the OD₆₆₀ using a microplate reader (Synergy 2; BioTek Instruments, Winooski, Vt., USA). The minimum inhibitory concentration (MIC) was defined as the highest dilution of compounds that completely inhibits bacterial growth. To determine the minimum bactericidal concentration (MBC), aliquots (5 μL) from wells showing no growth were plated on sheep blood-supplemented (5% [v/v]) THB-HK agar plates, which were incubated for 5 days at 37° C. The MBC was defined as the highest dilution of compounds at which no colony formation occurred. All assays were performed in triplicate to ensure reproducibility.

Ex vivo periodontal plaque samples: Subgingival plaque samples were collected with a sterile curette from the deepest periodontal pocket (probing depth≥6 mm) of five patients who had received a diagnosis of moderate to severe periodontitis. Each sample was inoculated in complete periodontal culture medium (CPCM; 10 mL), whose composition is described in Table 1. Following growth for 24 h at 37° C. under anaerobic conditions, the culture was diluted in CPCM to obtain an OD₆₆₀ of 0.2. The MIC and MBC of the Dual Zinc plus Arginine aqueous solution and dentifrice were determined using a broth microdilution assay as described above for P. gingivalis.

Hemolytic Assay

Fresh sheep red blood cells (Nutri-B act, Terrebonne, QC, Canada) were harvested from heparinized whole blood by centrifugation (600×g for 5 min), washed three times in phosphate-buffered saline (PBS; pH 7.0), and suspended in PBS to a concentration of 2% (v/v). Equal volumes (1 mL) of red blood cells, P. gingivalis cells (OD₆₆₀=1.0 in PBS), and two-fold serial dilutions of the Dual Zinc plus Arginine aqueous solution or dentifrice were mixed together. PBS replaced the bacteria in the negative control. Following an incubation at 37° C. for 4 h, the mixtures were incubated at 4° C. for 1 h and were then centrifuged (10 000×g for 5 min) prior to recording the absorbance of the supernatants at 540 nm (A₅₄₀).

Proteolytic Assay

To determine the effects of the Dual Zinc plus Arginine aqueous solution and dentifrice on the proteinase activity of P. gingivalis, a 48-h culture was centrifuged at 10 000×g for 10 min. Assay mixtures containing equal volumes of P. gingivalis culture supernatant, the fluorescent substrate collagen DQ™ (100 μg/mL; Molecular Probes, Eugene, Oreg., USA), and the Dual Zinc plus Arginine aqueous solution or dentifrice were prepared and incubated for 2 h at 37° C. The fluorescence corresponding to collagen degradation was monitored using a Synergy 2 microplate reader, with the excitation and emission wavelengths set at 495 nm and 525 nm, respectively. Test compounds or the fluorescent substrate alone were used as controls. Leupeptin (1 μM) was used as a positive inhibitory control.

Human Gingival Keratinocyte Culture

B11 immortalized human gingival keratinocyte cell line was used to investigate the effects of the Dual Zinc plus Arginine aqueous solution and dentifrice on keratinocyte barrier integrity. Keratinocytes were cultivated in keratinocyte serum-free medium (K-SFM; Life Technologies Inc., Burlington, ON, Canada) supplemented with growth factors (50 μg/mL of bovine pituitary extract and 5 ng/mL of human epidermal growth factor) and 100 μg/mL of penicillin G-streptomycin at 37° C. in a 5% CO₂atmosphere.

Transepithelial Electrical Resistance Assay

The tight junction integrity of the B11 gingival keratinocytes was assessed by determining the transepithelial electrical resistance (TER). Briefly, B11 keratinocytes were seeded onto Costar™ Transwell™ clear polyester membrane inserts (6.5-mm diameter; 0.4-μm pore size; Corning Co., Cambridge, Mass., USA) at 3×10⁵ cells per insert. The basolateral and apical compartments were filled with 0.6 mL and 0.1 mL of complete K-SFM, respectively, and the cultures were incubated for 3 days at 37° C. in a 5% CO₂ atmosphere. The conditioned medium was then replaced with fresh antibiotic-free K-SFM. Following a further incubation (16 h), the TER values were measured using an Ohm/voltmeter (EVOM2; World Precision Instruments, Sarasota, Fla., USA) at 0, 2, 6, 24, and 48 h. Resistance values were calculated in Ohms (Ω)/cm² by multiplying the resistance values by the filter surface area. Results were expressed as a percentage of the basal control values measured at time 0 h (100% values) for each condition. To investigate the effects of the Dual Zinc plus Arginine aqueous solution and dentifrice on tight junction integrity, the medium in the apical compartment was supplemented with the test compounds. The effect of these treatments on cell viability was assessed using an MTT (3-[4,5-diethylthiazol-2-yl]-2,5diphenyltetrazolim bromide) colorimetric assay, according to the manufacturer's instructions (Roche Diagnostics, Laval, QC, Canada).

The effect of P. gingivalis on the tight junction integrity of the gingival keratinocyte model was evaluated by monitoring TER at 0, 6, 24, and 48 h. P. gingivalis cells in antibiotic-free K-SFM were added to the apical compartment at a multiplicity of infection (MOI) of 10⁴. The protective effects of adding the Dual Zinc plus Arginine aqueous solution or dentifrice were evaluated by adding them to the apical compartment at the same time as the P. gingivalis cells.

Paracellular Permeability Assay

The ability of the test compounds to enhance or protect gingival keratinocyte barrier integrity was further assessed by monitoring the paracellular transport of fluorescein isothiocyanate (FITC)-conjugated 4.4-kDa dextran (FD-4; Sigma-Aldrich Canada Co., Oakville, ON, Canada) across the keratinocyte layer using the protocol described by Khan et al. (2015). Briefly, B11 cells were cultured on Transwell™ filters, and FD-4 (1 mg/mL in culture medium) was added to the apical compartment in the presence of the test compounds. The presence of FD-4 in the basolateral compartment was determined at 0, 2, 6, 24, and 48 h by measuring the fluorescence (RUF; excitation wavelength 495 nm; emission wavelength 525 nm) using a Synergy 2 microplate reader. The effects of P. gingivalis (MOI=10⁴) on paracellular permeability and the protective effects of the Dual Zinc plus Arginine aqueous solution and dentifrice were assessed under the conditions described above.

Immunofluorescent Staining of Zonula Occludens-1 and Occludin

Gingival keratinocytes treated for 48 h as described above (test compounds±P. gingivalis) were immunostained for two tight junction proteins (zonula occludens-1 and occludin). The localization of the tight junction proteins in B11 cells was visualized using an Olympus FSX100 fluorescence microscope and FSX-BSW imaging software (Olympus, Tokyo, Japan).

P. gingivalis Translocation Assay

B11 gingival keratinocytes cultured as described above were seeded at 2.25×10⁵ cells per insert in high-throughput screening (HTS) 96-well Costar™ Transwell™ plates (8-μm pore size; Corning Co.), which were placed in Costar™ black receiver plates (Corning Co.). The basolateral and apical compartments were filled with 0.235 mL and 0.075 mL of K-SFM, respectively. Following a 48-h incubation, the conditioned medium was replaced with antibiotic-free K-SFM. To determine the ability of P. gingivalis to penetrate the keratinocyte layer, FITC-labeled bacteria suspended in antibiotic-free K-SFM were added to the apical compartment of the double-chamber system at an MOI of 10⁴. Bacteria from an overnight culture were labeled with FITC as described previously (Marquis et al. 2012). To evaluate the effect of the Dual Zinc plus Arginine aqueous solution and dentifrice on the invasive capacity of P. gingivalis, the keratinocyte layer was co-incubated with them and the bacteria. The translocation of FITC-labeled bacteria through the keratinocyte barrier was monitored using a Synergy 2 microplate reader by measuring the fluorescence (RUF; excitation wavelength 495 nm; emission wavelength 525 nm) in the medium recovered from the lower chamber following a 24-h incubation in an anaerobic chamber at 37° C.

Statistical Analysis

Unless indicated otherwise, all experiments were performed in triplicate in three independent experiments. The data are expressed as means±standard deviations (SD). Statistical analyses were performed using a one-way analysis of variance with a post hoc Bonferroni multiple comparison test (GraphPad Software Inc., La Jolla, Calif., USA). All results were considered statistically significant at p<0.01 or p<0.001.

Results

The MIC and MBC of the Dual Zinc plus Arginine aqueous solution against P. gingivalis corresponded to a 1/125 dilution of the initial stock solution containing 0.96% zinc and 1.5% arginine (Table 2 below). The MIC and MBC values of the Dual Zinc plus Arginine dentifrice and the control fluoride dentifrice against P. gingivalis corresponded to a 1/1000 dilution. Ex vivo periodontal plaque samples from patients with moderate to severe periodontitis were used to further investigate the antibacterial activity of the Dual Zinc plus Arginine formulation. With MIC and MBC in the range of dilutions 1/62.5 to 1/125, the antibacterial activity of the Dual Zinc plus Arginine dentifrice against five periodontal plaque samples was more important than that of the Dual Zinc plus Arginine aqueous solution. As seen with P. gingivalis, the control regular fluoride dentifrice also exhibited some antibacterial activity against the ex vivo periodontal plaque samples.

P. gingivalis caused marked hemolysis of sheep red blood cells in a hemolytic assay (Table 3 below). Both the Dual Zinc plus Arginine aqueous solution and dentifrice inhibited hemolysis. At the lowest dilution tested (1/500), hemolysis was inhibited by 47.5% and 37.6%, respectively. The control fluoride dentifrice did not inhibit the hemolysis caused by P. gingivalis.

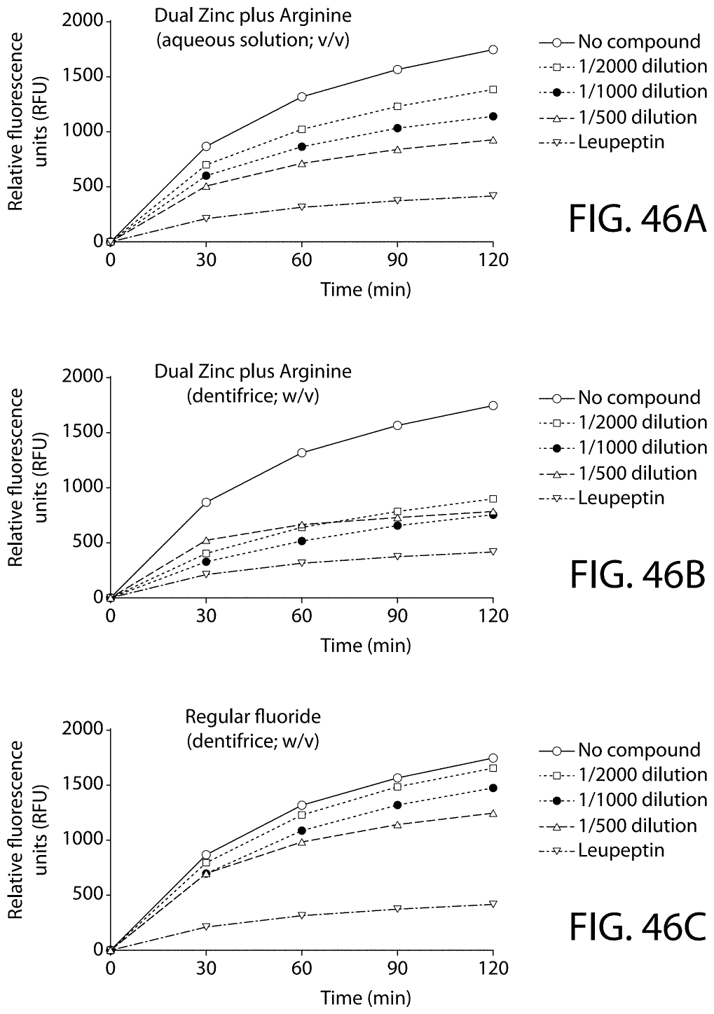

The ability of the Dual Zinc plus Arginine formulation (aqueous solution and dentifrice) to inhibit the degradation of type I collagen by proteinases in a culture supernatant of P. gingivalis was investigated. Significant time- and dose-dependent inhibition was observed with both the Dual Zinc plus Arginine aqueous solution and dentifrice (FIGS. 46A, 46B and 46C). More specifically, at the lowest dilution tested (1/500) and after a 2-h incubation, the Dual Zinc plus Arginine aqueous solution (FIG. 46A) and the Dual Zinc plus Arginine dentifrice (FIG. 46B) resulted in a 47.2% and 54.8% inhibition of type I collagen degradation, respectively. The control fluoride dentifrice reduced collagen degradation by 28.2% (FIG. 46C).

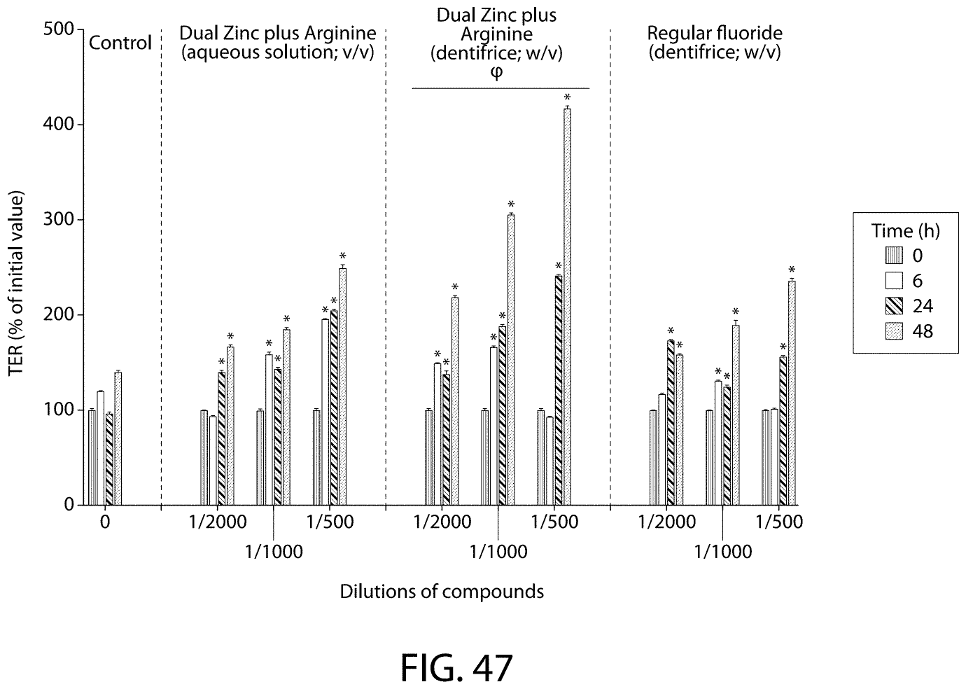

After investigating the effects of the Dual Zinc plus Arginine formulation (aqueous solution and dentifrice) on P. gingivalis, its ability to promote gingival epithelial barrier integrity was assessed. Preliminary assays showed that, at the concentrations used, the Dual Zinc plus Arginine aqueous solution and dentifrice did not significantly affect the viability of gingival keratinocytes as determined using a colorimetric MTT assay (data not shown). The ability of the Dual Zinc plus Arginine formulation to modulate the integrity of the gingival keratinocyte tight junction was determined by monitoring TER values over a period of 48 h. As shown in FIG. 47, the Dual Zinc plus Arginine aqueous solution and dentifrice induced a significant time-dependent increase in TER. A 24-h treatment of the keratinocytes with the 1/500 and 1/1000 dilutions of the Dual Zinc plus Arginine aqueous solution caused a 2.11- and 1.48-fold increase in TER, respectively, compared to untreated cells. A similar treatment with the Dual Zinc plus Arginine dentifrice caused a 2.49- and 1.93-fold increase in TER, respectively. Under the same conditions, the 1/500 and 1/1000 dilutions of the control fluoride dentifrice caused a 1.62- and 1.29-fold increase in TER, respectively.

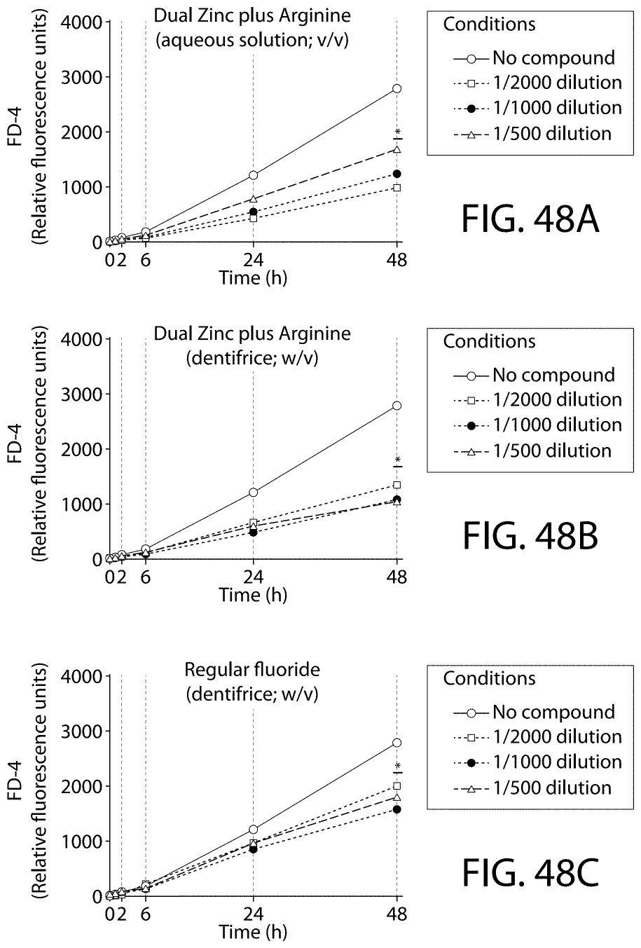

To confirm that the Dual Zinc plus Arginine aqueous solution and dentifrice enhanced the function of the keratinocyte barrier, their effect on paracellular permeability was investigated by measuring the apical-to-basolateral transport of FITC-dextran. As shown in FIGS. 48A, 48B and 48C, the paracellular transport of FITC-dextran time-dependently increased in the control (no compounds). However, in the presence of the Dual Zinc plus Arginine aqueous solution (FIG. 48A) or dentifrice (FIG. 48B), the increase in FITC-dextran transport through the gingival keratinocyte barrier was significantly attenuated. More specifically, following a 24-h treatment, the aqueous solution (FIG. 48A) and dentifrice (FIG. 48B) at the lowest dilution tested (1/500) reduced FITC-dextran transport by 36.4% and 49.0%, respectively, while the control fluoride dentifrice (FIG. 48C) only reduced FITC-dextran transport through the barrier model by 15.8%.

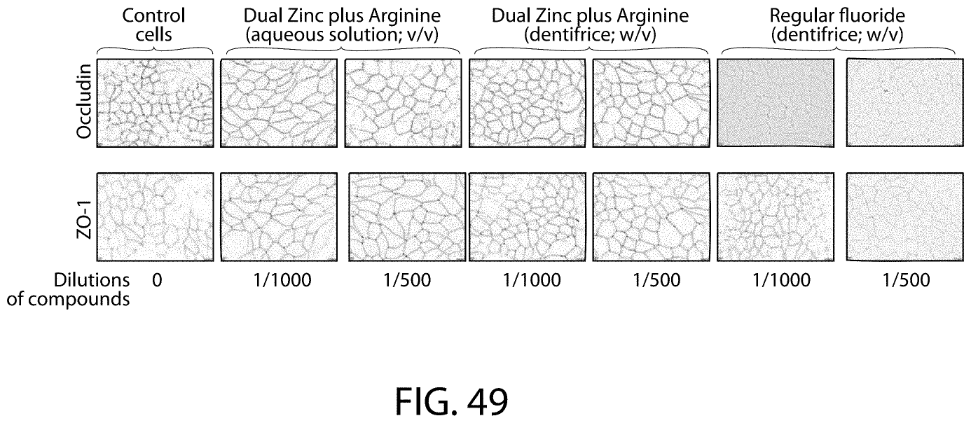

The effect of the 1/500 and 1/1000 dilutions of the Dual Zinc plus Arginine aqueous solution and dentifrice on the distribution of two junction proteins (Z0-1 and occludin) by immunofluorescence was examined. Both the aqueous solution and dentifrice increased the immunolabeling of ZO-1 and occludin in the areas of cell-cell contact (FIG. 49), while the regular fluoride dentifrice had no effect on the immunolabeling of ZO-1 and occludin.

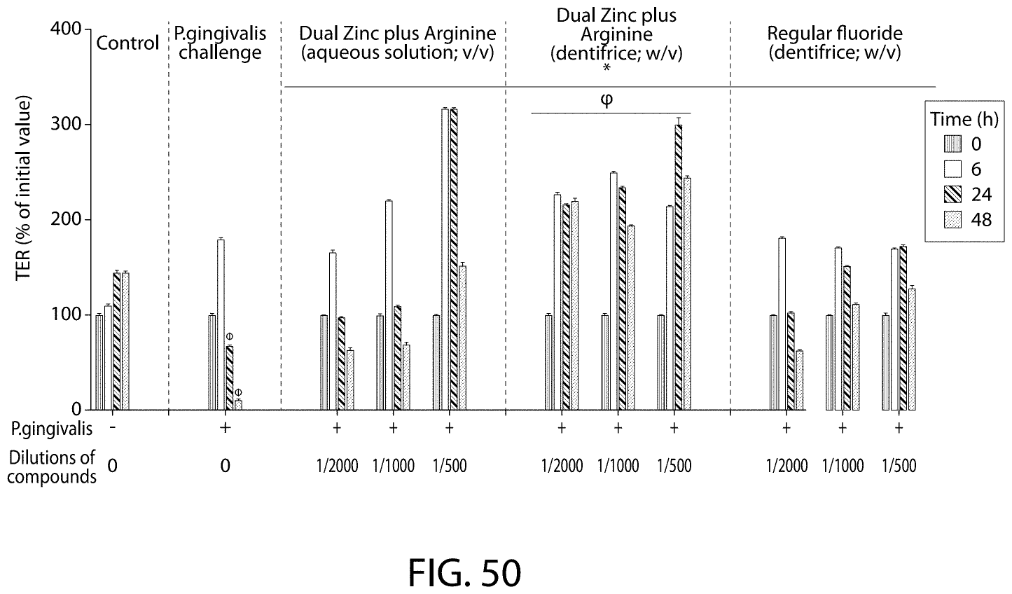

Since P. gingivalis may have a deleterious effect on keratinocyte barrier integrity, we investigated whether the Dual Zinc plus Arginine aqueous solution and dentifrice protect gingival keratinocytes from damage. Treating the keratinocytes with P. gingivalis at an MOI of 10⁴ significantly decreased TER. After 24- and 48-h incubations, P. gingivalis decreased TER by 53.2% and 92.1%, respectively (FIG. 50). Despite the effect of P. gingivalis on barrier integrity, it should be noted that no significant loss of cell viability was observed using an MTT assay that determines cell metabolic activity (data not shown). We then examined the protective effect of the Dual Zinc plus Arginine aqueous solution and dentifrice on TER when the keratinocytes were challenged with P. gingivalis. As shown in FIG. 50, both compounds attenuated the P. gingivalis-mediated loss of keratinocyte barrier integrity. More specifically, following a 48-h incubation, a 1/500 dilution of the Dual Zinc plus Arginine aqueous solution and dentifrice reduced the ability of P. gingivalis to decrease TER 13.4-fold and 21.4-fold, respectively. A 1/500 dilution of the regular fluoride dentifrice also provided a protective effect, reducing P. gingivalis-induced damage by 11.24-fold.

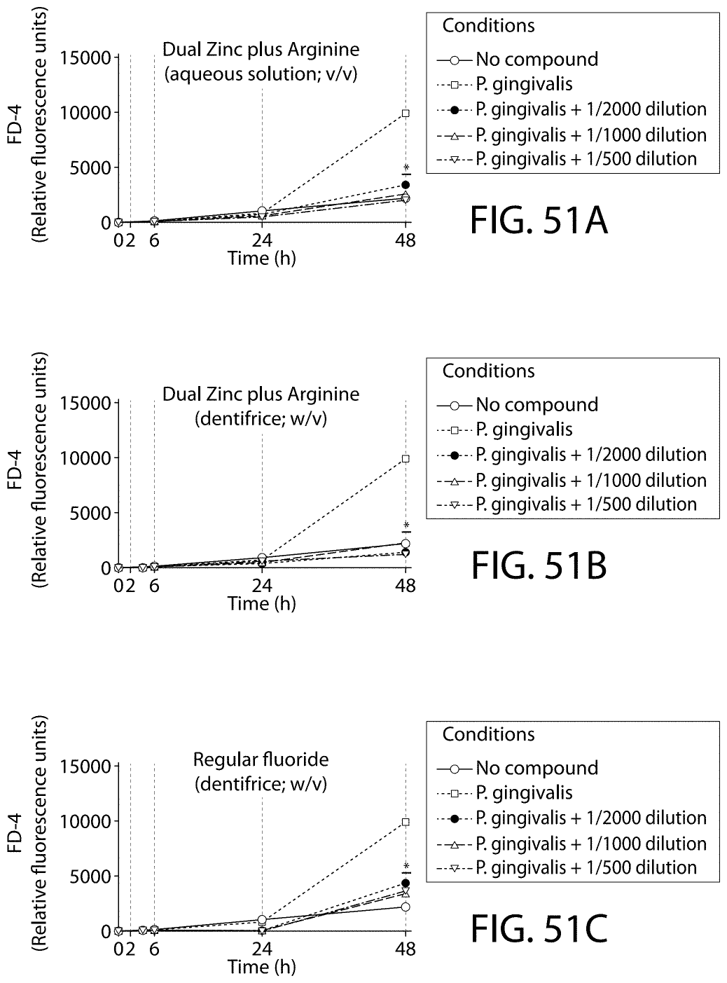

To confirm this protective effect, the effect of the Dual Zinc plus Arginine aqueous solution and dentifrice on P. gingivalis-induced paracellular flux of FD-4 through the keratinocyte barrier was investigated (FIGS. 51A, 51B and 51C). A 48-h treatment with a 1/500 dilution of the Dual Zinc plus Arginine aqueous solution (FIG. 51A) and dentifrice (FIG. 51B) caused a 4.92-fold and 7.62-fold decrease in FD-4 transport, respectively. The regular fluoride dentifrice caused a 2.67-fold decrease in FD-4 transport (FIG. 51C).

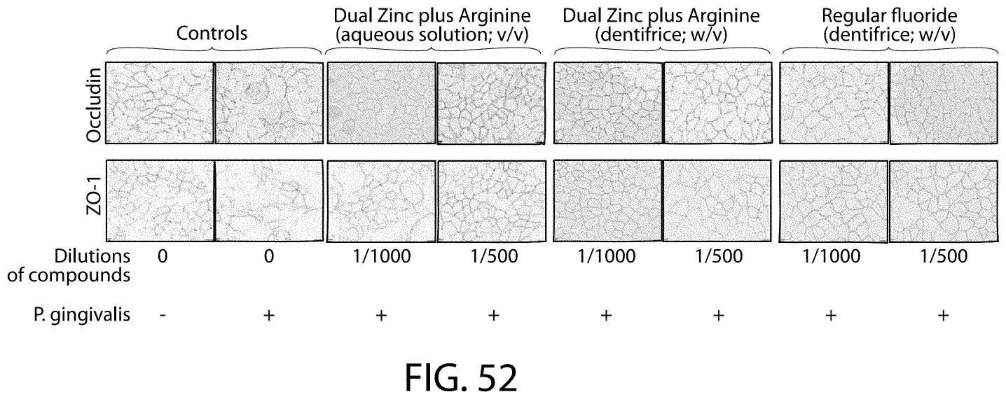

ZO-1 and occludin immunostaining was performed to determine whether P. gingivalis affects the keratinocyte barrier through the disruption of these two tight junction proteins. As shown in FIG. 52, a 48-h treatment of the keratinocytes with P. gingivalis (MOI of 10⁴) was associated with a marked decrease in ZO-1 and occludin immunolabeling. ZO-1 and occludin immunolabeling appeared to be less intense and more discontinuous in the cell-cell contacts after a treatment with P. gingivalis compared to control cells. However, both the Dual Zinc plus Arginine aqueous solution and dentifrice prevented the discontinuous and less intense immunolabeling of ZO-1 and occludin.

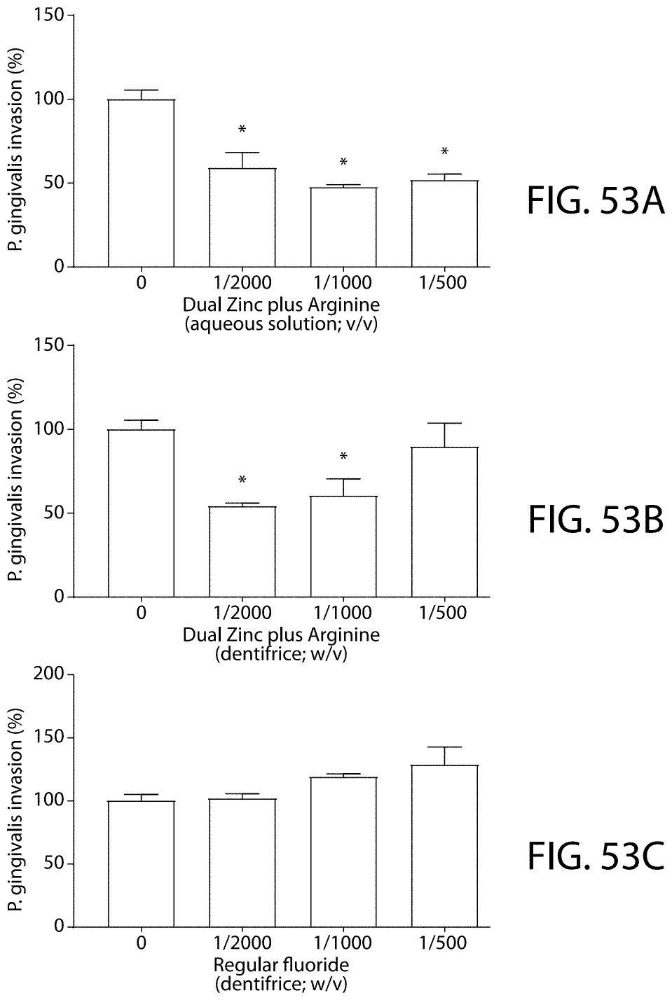

The effect of the Dual Zinc plus Arginine aqueous solution and dentifrice on the translocation of P. gingivalis through the gingival keratinocyte barrier model was determined. The FITC-labeled P. gingivalis cells crossed the keratinocyte barrier in a double-chamber system. As shown in FIGS. 53A, 53B and 53C, a 1/1000 dilution of the Dual Zinc plus Arginine aqueous solution (FIG. 53A) and dentifrice (FIG. 53B) significantly reduced the migration of the FITC-labeled P. gingivalis cells through the barrier by 53.0% and 39.1%, respectively. The regular fluoride dentifrice caused no significant decrease in the migration of FITC-labeled P. gingivalis cells (FIG. 53C).

### Discussion

Two strategies can be used to promote periodontal health: (1) eliminate/neutralize periodontal pathogens, and (2) improve innate immunity by reinforcing epithelial barrier function. In the present study, the effects of a Dual Zinc plus Arginine aqueous solution and dentifrice on the growth and pathogenic properties of P. gingivalis as well as on the barrier function of an in vitro gingival epithelium model was investigated.

The Dual Zinc plus Arginine formulation showed antibacterial activity against P. gingivalis. Moreover, both the aqueous solution and the dentifrice inhibited the growth of ex vivo periodontal plaque samples. The antibacterial activity of the Dual Zinc plus Arginine formulation likely relies on the presence of zinc (oxide and citrate). On the one hand, water-soluble zinc salts such as zinc citrate act rapidly on bacteria by inactivating glycolytic and respiratory chain enzymes and by increasing the permeability of bacterial membranes. On the other hand, zinc oxide is poorly soluble and may serve as a reservoir and exert its antibacterial activity over time. Although arginine is not known to possess antibacterial properties, it has been shown to enhance the bioavailability of the Dual Zinc system, thus facilitating the deposition, penetration, and retention of zinc ions in oral biofilms in in vitro models al. With regard to dental caries, arginine has been reported to be metabolized by the arginine deiminase pathway of specific bacterial species, resulting in ammonia production that counteracts cariogenic biofilm acidification.

In the present study, Dual Zinc plus Arginine formulation was shown to reduce the hemolytic activity of P. gingivalis. The ability of P. gingivalis to lyse erythrocytes and release hemoglobin is considered a virulence determinant since it provides an iron source to P. gingivalis and other periodontal pathogens that promotes their proliferation in subgingival sites. Moreover, hemoglobin has been reported to synergize with P. gingivalis lipopolysaccharides to amplify the inflammatory response of human macrophages. As such, the inhibition of hemolysis by the Dual Zinc plus Arginine formulation may contribute to reducing the levels of pro-inflammatory mediators in periodontal sites in addition to attenuate growth of P. gingivalis.

Type I collagen makes up approximately 60% of the tissue volume of periodontal tissues. The collagenolytic activity of P. gingivalis has been attributed to the action of its gingipains, which are both secreted and cell-bound. Dual Zinc plus Arginine formulation dose-dependently inhibits collagen degradation by P. gingivalis, suggesting that it may contribute to reducing the tissue destructive process mediated by this periodontal pathogen. Both L-arginine and zinc, both of which are found in the Dual Zinc plus Arginine formulation, are highly effective in this regard.

The first line of host defense against both opportunistic and pathogenic microorganisms colonizing the oral cavity is the oral epithelium. The physical epithelial barrier is composed of closely opposed cells that connect neighboring cells to each other by specialized intercellular tight junctions. These tight junctions seal the paracellular space, blocking the pathway to bacteria and toxins while allowing the flux of water and nutrients. Given the crucial protective role played by the oral epithelial barrier, compounds endowed with a capacity to enhance or protect tissue barrier function are of great interest as potential oral care products. Plant polyphenols, including green tea catechins, black tea theaflavins and blueberry proanthocyanidins improve tight junction integrity in an in vitro gingival epithelium model. Dual Zinc plus Arginine aqueous solution and dentifrice significantly enhance the barrier function of a gingival keratinocyte model, as determined by a time-dependent increase in transepithelial electrical resistance and decrease in paracellular permeability. Moreover, the Dual Zinc plus Arginine formulation also increased the immunolabelling of ZO-1 and occludin. Dual Zinc plus Arginine aqueous solution and dentifrice did not upregulate gene expression of these two major tight junction proteins (data not shown). The ability of the Dual Zinc plus Arginine formulation to promote gingival keratinocyte barrier function may be rather associated with the presence of zinc.

In addition to being a physical barrier against the invasion of the underlying connective tissue by periodontopathogenic bacteria, keratinocytes provide an immunological barrier by secreting antimicrobial β-defensin peptides. Green tea catechins can increase the innate immunity of oral keratinocytes by inducing human β-defensin secretion.

P. gingivalis has developed various strategies to invade the gingival epithelium and overcome its protective functions. P. gingivalis can compromise epithelial barrier function by inducing the disorganization of cell-cell interactions as shown by the decrease in TER and increase in FD-4 transport. P. gingivalis also affected the distribution of two major tight junction proteins (zonula occludens-1 and occludin). Western blotting was used to show that P. gingivalis (cells and supernatant) can cleave purified occludin. These effects may allow bacteria to reach and damage the underlying connective tissue. The Dual Zinc plus Arginine formulation protected the gingival keratinocyte barrier against P. gingivalis-mediated damage. This protective effect may rely on the ability of the zinc to enhance the gingival epithelium barrier function observed in the present study. It may also, at least in part, result from the ability of the Dual Zinc plus Arginine formulation to inhibit P. gingivalis gingipain activity. These proteolytic enzymes may be involved in the degradation of cell-to-cell junctions and the disruption of the epithelial barrier. In vivo (mouse) and in vitro models demonstrate that zinc can protect the intestinal epithelial barrier from damage induced by the pathogen Shigella flexneri. This protective effect was associated with a redistribution of two tight junction proteins (claudin-2 and -4) to the plasma membrane.

The intercellular spaces of the stratified oral epithelium offer a pathway for P. gingivalis to invade tissues during periodontitis. The effect of the Dual Zinc plus Arginine formulation on the translocation of P. gingivalis through an in vitro model of the gingival epithelium was investigated. FITC-labeled bacteria show that the formulation reduced the migration of P. gingivalis through the gingival keratinocyte barrier in a double-chamber system.

In conclusion, under the assay conditions of in vitro models, data indicates that Dual Zinc plus Arginine formulation, in an aqueous solution or in a dentifrice, may offer benefits for patients affected by periodontal disease through its ability to exert antibacterial activity, attenuate the pathogenic properties of P. gingivalis, and enhance epithelial barrier function.

TABLE 1
Composition of complete periodontal culture medium (CPCM).

| Ingredients | Amount | |
|---|---|---|
| Todd-Hewitt Broth | 20 | g |
| Brain Heart Infusion Broth | 12 | g |
| Trypticase Soy Broth | 10 | g |
| Yeast extract | 10 | g |
| Cysteine hydrochloride | 1 | g |
| Sodium thioglycolate | 0.5 | g |
| L-asparagine | 0.25 | g |
| Hemin | 10 | mg |
| Vitamin K | 1 | mg |
| Autoclave and add filter-sterilized solutions of: | | |
| Glucose 20% | 5 | mL |
| Sodium bicarbonate 10% | 10 | mL |
| Thiamine pyrophosphate 0.2% | 1.5 | mL |
| N-acetyl muramic acid 1% | 1 | mL |
| Volatile fatty acids* | 1 | mL |
| Fetal bovine serum (heat-inactivated) | 5 | mL |
| Horse serum (heat-inactivated) | 10 | mL |

*The stock solution of volatile fatty acids contains 0.5 mL each of isobutyric, DL-2-methylbutyric, isovaleric, and valeric acids in 100 mL of 0.1N KOH.

TABLE 2
Minimum inhibitory concentrations (MIC) and minimum
bactericidal concentrations (MBC) of the Dual Zinc plus Arginine
aqueous solution, Dual Zinc plus Arginine dentifrice,
and control fluoride dentifrice against
P. gingivalis and ex vivo periodontal plaque samples.

| | Compounds | | | | | |
|---|---|---|---|---|---|---|
| | Dual Zinc plus | | Dual Zinc plus | | Control | |
| | Arginine aqueous | | Arginine | | fluoride | |
| | solution | | dentifrice | | dentifrice | |
| | MIC | MBC | MIC | MBC | MIC | MBC |
| P. gingivalis | 1/125 | 1/125 | 1\1000 | 1/1000 | 1/1000 | 1/1000 |
| Ex vivo | <1/15.625 | <1/15.625 | 1/62.5 | 1/62.5 | 1/31.25 | 1/31.25 |
| plaque | | | | | | |
| sample #1 | | | | | | |
| Ex vivo | 1/15.625 | <1/15.625 | 1/62.5 | 1/62.5 | 1/62.5 | 1/62.5 |
| plaque | | | | | | |
| sample #2 | | | | | | |
| Ex vivo | <1/15.625 | <1/15.625 | 1/62.5 | 1/62.5 | 1/62.5 | 1/62.5 |
| plaque | | | | | | |
| sample #3 | | | | | | |
| Ex vivo | <1/15.625 | <1/15.625 | 1/125 | 1/62.5 | 1/62.5 | 1/62.5 |
| plaque | | | | | | |
| sample #4 | | | | | | |
| Ex vivo | <1/15.625 | <1/15.625 | 1/125 | 1/62.5 | 1/125 | 1/31.25 |
| plaque | | | | | | |
| sample #5 | | | | | | |

TABLE 3
Effects of the Dual Zinc plus Arginine aqueous solution, the Dual
Zinc plus Arginine dentifrice, and the regular fluoride dentifrice
on the hemolytic activity of P. gingivalis. A value
of 100% was assigned to the hemolysis induced by P. gingivalis
in the absence of compounds. Results are expressed as
the means ± SD of triplicate assays. *, significant inhibition
(p <0.01) compared to control (no compounds).

| Compounds | Dilution | Relative hemolysis |
|---|---|---|
| None | | 100% |
| Dual Zinc plus Arginine | 1/500 | 52.5 ± 1.7%* |
| aqueous solution | 1/1000 | 57.2 ± 0.6%* |
| | 1/2000 | 54.3 ± 2.9%* |
| | 1/4000 | 61.1 ± 0.5%* |
| | 1/8000 | 87.5 ± 2.2%* |
| | 1/16000 | 104.6 ± 0.9% |
| Dual Zinc plus Arginine dentifrice | 1/500 | 62.4 ± 1.2%* |
| | 1/1000 | 61.1 ± 0.5%* |
| | 1/2000 | 69.2 ± 0.1%* |
| | 1/4000 | 74.0 ± 1.2%* |
| | 1/8000 | 85.6 ± 3.3%* |
| | 1/16000 | 105.7 ± 1.2% |
| Control fluoride dentifrice | 1/500 | 114.7 ± 2.3% |
| | 1/1000 | 94.4 ± 1.9% |
| | 1/2000 | 106.7 ± 4.3% |
| | 1/4000 | 106.4 ± 0.8% |
| | 1/8000 | 98.6 ± 4.9% |
| | 1/16000 | 92.4 ± 1.6% |

### Example 8

Oral compositions that comprise arginine are disclosed in WO 2015/094849, which corresponds to US 2016/0338921, which are both incorporated herein by reference. In some embodiments the oral care composition comprises: arginine, in free or salt form; and zinc oxide and zinc citrate. In some embodiments, the arginine is present in an amount of 0.5 weight % to 3 weight %, such as 1 weight % to 2.85 weight %, such as 1.17 weight % to 2.25 weight %, such as 1.4 weight % to 1.6 weight %, such as about 1.5 weight %, based on the total weight of the composition. In some embodiments set out above, the total concentration of zinc salts in the composition is 0.2 weight % to 5 weight %, based on the total weight of the composition. In some embodiments set out above, the molar ratio of arginine to total zinc salts is 0.05:1 to 10:1. In some embodiments set out above, the composition comprises zinc oxide in an amount of 0.5 weight % to 1.5 weight %, such as 1 weight %, and zinc citrate in an amount of 0.25 weight % to 0.75 weight %, such as 0.5 weight %, based on the total weight of the composition. In some embodiments set out above, the weight ratio of zinc oxide to zinc citrate is 1.5:1 to 4.5:1, optionally 1.5:1 to 4:1, 1.7:1 to 2.3:1, 1.9:1 to 2.1:1, or about 2:1.

### Example 9

Oral compositions that comprise arginine are disclosed in WO 2017/003844, which corresponds to US 2018/0021234, which are both incorporated herein by reference. In some embodiments, the oral care composition comprises: arginine, zinc oxide and zinc citrate and a fluoride source. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly salt form. In some embodiments set out above, the ratio of the amount of zinc oxide (by wt %) to zinc citrate (by wt %) is 2:1, 2.5:1, 3:1, 3.5:1 or 4:1, wherein the ratio is by wt. of the overall composition. In some embodiments, the zinc citrate is in an amount of from 0.25 to 1.0 wt % and zinc oxide may be present in an amount of from 0.75 to 1.25 wt % or the zinc citrate is in an amount of about 0.5 wt % and zinc oxide is present in an amount of about 1.0%, based on the total weight of the composition. In some embodiments set out above, the fluoride source is sodium fluoride or sodium monofluorophosphate. In some such embodiments, the sodium fluoride or sodium monofluorophosphate is from 0.1 wt. %-2 wt. % based on the total weight of the composition. In some embodiments, the sodium fluoride or sodium monofluorophosphate is a soluble fluoride salt which provides soluble fluoride in amount of 50 to 25,000 ppm fluoride, such as in an amount of about 1000 ppm-1500 ppm, for example in an amount of about 1450 ppm. In some embodiments the fluoride source is sodium fluoride in an amount about 0.32% by wt, based on the total weight of the composition. In some embodiments, the fluoride source is stannous fluoride. Some embodiments set out above further comprise a preservative selected from: benzyl alcohol, Methylisothizolinone (“MIT”), Sodium bicarbonate, sodium methyl cocoyl taurate (tauranol), lauryl alcohol, and polyphosphate. Some embodiments set out above further comprise benzyl alcohol in an amount of from 0.1-0.8% wt %, or from 0.3-0.5% wt %, or about 0.4 wt % based on the total weight of the composition. In some embodiments, the oral care composition comprises about 1.0% zinc oxide, about 0.5% zinc citrate, about 1.5% L-arginine, about 1450 ppm sodium fluoride, and optionally about benzyl alcohol 0.1 wt. % and/or about 5% small particle silica (e.g., AC43), based on the total weight of the composition. In some embodiments, the oral care composition comprises about 1.0% zinc oxide, about 0.5% zinc citrate, about 5% L-arginine, about 1450 ppm sodium fluoride, and optionally about benzyl alcohol 0.1 wt. % and/or about 5% small particle silica (e.g., AC43), based on the total weight of the composition. In some embodiments set out above, the oral care composition may comprise about 1.0% zinc oxide, about 0.5% zinc citrate, about 1.5% L-arginine, about 0.22%-0.32% sodium fluoride, about 0.5% tetrasodium pyrophosphate, and optionally about benzyl alcohol 0.1 wt. %, based on the total weight of the composition. In some embodiments set out above, the oral care composition may be any of the following oral care compositions selected from the group consisting of: a toothpaste or a dentifrice, a mouthwash or a mouth rinse, a topical oral gel, and a denture cleanser.

### Example 10

Oral compositions that comprise arginine are disclosed in WO 2017/223169, which is incorporated herein by reference. In some embodiments, the oral care composition comprises: arginine in free or salt form, zinc oxide and zinc citrate and a fluoride source comprising stannous fluoride. In some embodiments, the oral care compositions comprise zingerone, zinc oxide, zinc citrate; and a stannous fluoride. In some embodiments, the zingerone is present in an amount of from 0.01% to 1%, based on the total weight of the composition. In some embodiments, the ratio of the amount of zinc oxide (by wt %) to zinc citrate (by wt %) is 2:1, 2.5:1, 3:1, 3.5:1 or 4:1, based on the total weight of the composition. In some embodiments, the zinc citrate is present in an amount of from 0.25 to 1.0 wt % and zinc oxide is present in an amount of from 0.75 to 1.25 wt %, based on the total weight of the composition. In some embodiments, the zinc citrate is present in an amount of about 0.5 wt % and zinc is present in an amount of about 1.0% based on the total weight of the composition. In some embodiments, the stannous fluoride is present in an amount of 0.1 wt, % to 2 wt. %, based on the total weight of the composition. Some embodiments further comprise synthetic amorphous precipitated abrasive silica in an amount of from 1%-25% by wt, based on the total weight of the composition and/or a high cleaning silica in an amount of from 1 wt %—15 wt %, based on the total weight of the composition. Some embodiments further comprise an effective amount of one or more alkali phosphate salts, for example sodium tripolyphosphate in an amount of from 1-5 wt %, based on the total weight of the composition. Some embodiments further comprise citric acid in an amount of from 0.1-3 wt. %, and citrate ion, for example trisodium citrate dihydrate, in an amount of from 0.1-5 wt. %, based on the total weight of the composition. Some embodiments further comprise carboxymethyl cellulose in an amount of from 0.1 wt, %-1.5 wt. %, based on the total weight of the composition. Some embodiments further comprise an anionic surfactant, e.g., sodium lauryl sulfate, in an amount of from 0.5-5% by weight, based on the total weight of the composition. Some embodiments further comprise an amphoteric surfactant in an amount of from 0.5-5%, based on the total weight of the composition. Some embodiments further comprise a PVM/MA copolymer, such as for example a Gantrez polymer, in an amount of from 0.1-5 wt. %, based on the total weight of the composition. Some embodiments further comprise microcrystalline cellulose/sodium carboxymethylcellulose. Some embodiments further comprise one or both of polyethylene glycol in an amount of from 1-6%; and propylene glycol in an amount of from 1-6%, based on the total weight of the composition. Some embodiments further comprise polyvinylpyrrolidone (PVP) in an amount of from 0.5-3 wt. %, based on the total weight of the composition. Some embodiments further comprise from 5%-40% free water by weight, based on the total weight of the composition. Some embodiments further comprise one or more thickening agents, e.g. sodium carboxymethyl cellulose and sodium carboxy methyl hydroxyethyl cellulose. In some embodiments, the oral care composition comprises: about 0.1-0.3% zingerone; about 1.0% zinc oxide; about 0.5% zinc citrate, and about 0.4%-0.5% stannous fluoride. In some embodiments, the oral care composition comprises: about 0.1-0.3% zingerone; about 1.0% zinc oxide; about 0.5% zinc citrate, about 0.4%-0.5% stannous fluoride; and about 1.2% abrasive silica and may, in some such embodiments, further comprise about 7% wt % high cleaning silica, based on the total weight of the composition, and/or a surfactant system comprising one or both of an anionic surfactant in an amount of from 0.5-5%, by weight; and/or an amphoteric surfactant in an amount of from 0.5-5% by weight, based on the total weight of the composition. Some embodiments further comprise sodium tripolyphosphate in an amount of from 1-5 wt %, based on the total weight of the composition and/or sodium phosphate in an amount of from 0.5 wt %-5 wt %, based on the total weight of the composition. Examples of the oral composition include a toothpaste or a dentifrice, a mouthwash or a mouth rinse, a topical oral gel, a chewing gum, or a denture cleanser.

### Example 11

Test dentifrices comprising arginine, zinc oxide, zinc citrate and a source of fluoride were prepared as shown in Formulation Tables A-E:

Formulation Table A

| Ingredient | Composition 1 |
|---|---|
| Humectants | 20.0-25.0 |
| Non-ionic surfactant | 1.0-2.0 |
| Amphoteric surfactant | 3.0-4.0 |
| Flavoring/fragrance/coloring agent | 2.0-3.0 |
| Polymers | 10.0-15.0 |
| pH adjusting agents | 1.5-3.0 |
| Precipitated Calcium Carbonate | 35 |
| Zinc citrate trihydrate | 0.5 |
| Zinc oxide | 1.0 |
| Sodium Fluoride-USP, EP | 0.32 |
| Arginine Bicarbonate | 13.86 |
| Demineralized water | QS |

Formulation Table B

| | Com- | Com- | Com- | Com- |
|---|---|---|---|---|
| Ingredient | position A | pound B | position C | position D |
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/ | 2.5-4.0 | 2.5-4.0 | 2.5-4.0 | 2.5-4.0 |
| coloring agent | | | | |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous | 16.00 | 21.37 | 17.92 | 7.81 |
| Precipitated | | | | |
| Silica | | | | |
| Alumina | 0.02 | 0.01 | 0.01 | 0.01 |
| Silica | — | — | — | 15.0 |
| Lauryl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium Fluoride-USP, EP | 0.32 | 0.32 | 0.32 | 0.32 |
| L-Arginine Bicarbonate | 5.0 | 5.0 | 5.0 | 5.0 |
| Demineralized water | QS | QS | QS | QS |

Formulation Table C

| | Com- | Com- | Com- |
|---|---|---|---|
| Ingredient | position E | position F | position G |
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-1.0 | 0.1-1.0 |
| Flavoring/fragrance/ | 4.0-6.0 | 4.0-6.0 | 4.0-6.0 |
| coloring agent | | | |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7.0 |
| Alumina | 0.1 | 0.1 | 0.1 |
| Synthetic Amorphous | 17.6 | 8.8 | 22.4 |
| Precipitated | | | |
| Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.5 | 0.5 |
| Zinc oxide | 1.0 | 1.0 | 1.0 |
| Sodium Fluoride-USP, EP | 0.32 | 0.32 | 0.32 |
| L-Arginine Bicarbonate | 1.5 | 1.5 | 1.5 |
| Demineralized water | QS | QS | QS |

Formulation Table D

| Ingredient | Composition H | Composition I |
|---|---|---|
| Humectants | 45.0-55.0 | 35.0-45.0 |
| Abrasives | 14.0-16.0 | 9.0-11.0 |
| Anionic surfactant | 1.0-3.0 | 1.0-3.0 |
| Non-ionic surfactant | 0.1-1.0 | — |
| Amphoteric surfactant | 1.0-2.0 | — |
| Flavoring/fragrance/coloring agent | 1.0-3.0 | 2.0-4.0 |
| Polymers | 0.1-2.0 | 3.0-8.0 |
| pH adjusting agents | 0.1-2.0 | 4.0-8.0 |
| Silica Thickener | 5.0 | 5.0-10.0 |
| Benzyl alcohol | 0.1 | — |
| Zinc citrate trihydrate | 0.5 | 0.5 |
| Zinc oxide | 1.0 | 1.0 |
| Sodium Fluoride-USP, EP | 0.32 | 0.32 |
| L-Arginine | 1.5 | 5.0 |
| Demineralized water | QS | QS |

Formulation Table E

| | Com- | Com- | Com- |
|---|---|---|---|
| Ingredient | position J | position K | position L |
| Humectants | 20.0-50.0 | 20.0-50.0 | 20.0-50.0 |
| Abrasives | 5.0-20.0 | 5.0-20.0 | 5.0-20.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-2.0 | 0.1-2.0 | 0.1-2.0 |
| Flavoring/fragrance/ | 1.0-5.0 | 1.0-5.0 | 1.0-5.0 |
| coloring agent | | | |
| Polymers | 0.1-2.0 | 0.1-2.0 | 0.1-2.0 |
| pH adjusting agents | 0.1-2.0 | 0.1-2.0 | 0.1-2.0 |
| Thickener | 6.0 | 6.5 | 7.0 |
| Dental type silica | — | — | 15.0 |
| High cleaning silica | — | 15.0 | — |
| Synthetic Abrasives | 10.0 | — | — |
| Synthetic Amorphous Silica | 5.0 | 5.0 | 5.0 |
| Benzyl alcohol | 0.4 | 0.4 | 0.4 |
| Zinc citrate trihydrate | 0.5 | 0.5 | 0.5 |
| Zinc oxide | 1.0 | 1.0 | 1.0 |
| Sodium Fluoride-USP, EP | 0.32 | 0.32 | 0.32 |
| L-Arginine | 1.5 | 1.5 | 1.5 |
| Demineralized water | QS | QS | QS |

### Example 12

Test dentifrices comprising arginine, zinc oxide, zinc citrate and stannous fluoride were prepared as shown in Formulation Table F:

Formulation Table F

| Ingredient | Composition M | Composition N | Coinposition O |
|---|---|---|---|
| Humectants | 20.0-60.0 | 20.0-50.0 | 20.0-50.0 |
| Abrasives | 10.0-40.0 | 5.0-20.0 | 5.0-20.0 |
| Anionic surfactant | 1.0-3.0 | 1.0-3.0 | 1.0-3.0 |
| Amphoteric surfactant | 0.5-1.5 | 0.1-2.0 | 0.1-2.0 |
| Flavoring/fragrance/ | 0.5-5.0 | 1.0-5.0 | 1.0-5.0 |
| coloring agent | | | |
| Polymers | 1.0-10.0 | 0.1-2.0 | 0.1-2.0 |
| pH adjusting agents | 1.0-10.0 | 0.1-2.0 | 0.1-2.0 |
| Zinc citrate | 0.25-1.0 | 0.5 | 0.5 |
| Zinc oxide | 0.75-1.25 | 1.0 | 1.0 |
| Stannous Fluoride | 0.1-1.0 | 0.32 | 0.32 |
| L-Arginine | 0.1-10.0 | 1.5 | 1.5 |
| Demineralized water | QS | QS | QS |

### Example 13

Test dentifrices may be prepared comprising ingredients as shown in Formulation Tables G-P.

Formulation Table G

| | Com- | Com- | Com- | Com- |
|---|---|---|---|---|
| Ingredient | position P | position Q | position R | position S |
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/ | 2.5-4.0 | 2.5-4.0 | 2.5-4.0 | 2.5-4.0 |
| coloring agent | | | | |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous | 16.00 | 21.37 | 17.92 | 7.81 |
| Precipitated Silica | | | | |
| Alumina | 0.02 | 0.01 | 0.01 | 0.01 |
| Silica | — | — | — | 15.0 |
| Laulryl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium Fluoride-USP, EP | 0.32 | 0.32 | 0.32 | 0.32 |
| Glutamine | 0.5 | 0.5 | 0.5 | 0.5 |
| Demineralized water | QS | QS | QS | QS |

Formulation Table H

| | Com- | Com- | Com- | Com- |
|---|---|---|---|---|
| Ingredient | position P′ | position Q′ | position R′ | position S′ |
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/ | 2.5-4.0 | 2.5-4.0 | 2.5-4.0 | 2.5-4.0 |
| coloring agent | | | | |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous | 16.00 | 21.37 | 17.92 | 7.81 |
| Precipitated | | | | |
| Silica | | | | |
| Alumina | 0.02 | 0.01 | 0.01 | 0.01 |
| Silica | — | — | — | 15.0 |
| Lauryl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium | 0.32 | 0.32 | 0.32 | 0.32 |
| Fluoride-USP, EP | | | | |
| Glycine | 0.5 | 0.5 | 0.5 | 0.5 |
| Demineralized water | QS | QS | QS | QS |

Formulation Table I

| | Com- | Com- | Com- | Com- |
|---|---|---|---|---|
| Ingtedient | position P″ | position Q″ | position R″ | position S″ |
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/ | 2.5-4.0 | 2.5-4.0 | 2.5-4.0 | 2.5-4.0 |
| coloring agent | | | | |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous | 16.00 | 21.37 | 17.92 | 7.81 |
| Precipitated | | | | |
| Silica | | | | |
| Alumina | 0.02 | 0.01 | 0.01 | 0.01 |
| Silica | — | — | — | 15.0 |
| Lauryl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium | 0.32 | 0.32 | 0.32 | 0.32 |
| Fluoride-USP, EP | | | | |
| Asparagine | 0.5 | 0.5 | 0.5 | 0.5 |
| Demineralized water | QS | QS | QS | QS |

Formulation Table J

| | Com- | Com- | Com- |
|---|---|---|---|
| Ingredient | position T | position U | position V |
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-1.0 | 0.1-1.0 |
| Flavoring/fragrance/ | 4.0-6.0 | 4.0-6.0 | 4.0-6.0 |
| coloring agent | | | |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7.0 |
| Alumina | 0.1 | 0.1 | 0.1 |
| Synthetic Amorphous | 17.6 | 8.8 | 22.4 |
| Precipitated Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.5 | 0.5 |
| Zinc oxide | 1.0 | 1.0 | 1.0 |
| Sodium Fluoride-USP, EP | 0.32 | 0.32 | 0.32 |
| Glutamine | 1.5 | 1.5 | 1.5 |
| Demineralized water | QS | QS | QS |

Formulation Table K

| | Com- | Com- | Com- |
|---|---|---|---|
| Ingredient | position T′ | position U′ | position V′ |
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-1.0 | 0.1-1.0 |
| Flavoring/fragrance/ | 4.0-6.0 | 4.0-6.0 | 4.0-6.0 |
| coloring agent | | | |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7.0 |
| Alumina | 0.1 | 0.1 | 0.1 |
| Synthetic Amorphous | 17.6 | 8.8 | 22.41 |
| Precipitated Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.5 | 0.5 |
| Zinc oxide | 1.0 | 1.0 | 1.0 |
| Sodium Fluoride-USP, EP | 0.32 | 0.32 | 0.32 |
| Glutamine | 1.5 | 1.5 | 1.5 |
| Demineralized water | QS | QS | QS |

Formulation Table L

| | Com- | Com- | Com- |
|---|---|---|---|
| Ingredient | position T″ | position U″ | position V″ |
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-1.0 | 0.1-1.0 |
| Flavoring/fragrance/ | 4.0-6.0 | 4.0-6.0 | 4.0-6.0 |
| coloring agent | | | |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7.0 |
| Alumina | 0.1 | 0.1 | 0.1 |
| Synthetic Amorphous | 17.6 | 8.8 | 22.4 |
| Precipitated Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.5 | 0.5 |
| Zinc oxide | 1.0 | 1.0 | 1.0 |
| Sodium Fluoride-USP, EP | 0.32 | 0.32 | 0.32 |
| Asparagine | 1.5 | 1.5 | 1.5 |
| Demineralized water | QS | QS | QS |

Formulation Table M

| Ingredient | Composition W | Composition X | Composition Y | Composition Z |
|---|---|---|---|---|
| Humectants | 45.0-55.0 | 35.0-45.0 | 45.0-55.0 | 35.0-45.0 |
| Abrasives | 14.0-16.0 | 9.0-11.0 | 14.0-16.0 | 9.0-11.0 |
| Anionic surfactant | 1.0-3.0 | 1.0-3.0 | 1.0-3.0 | 1.0-3.0 |
| Non-ionic surfactant | 0.1-1.0 | — | 0.1-1.0 | — |
| Amphoteric surfactant | 1.0-2.0 | — | 1.0-2.0 | — |
| Flavoring/fragrance/coloring agent | 1.0-3.0 | 2.0-4.0 | 1.0-3.0 | 2.0-4.0 |
| Polymers | 0.1-2.0 | 3.0-8.0 | 0.1-2.0 | 3.0-8.0 |
| pH adjusting agents | 0.1-2.0 | 4.0-8.0 | 0.1-2.0 | 4.0-8.0 |
| Silica Thickener | 5.0 | 5.0-10.0 | 5.0 | 5.0-10.0 |
| Benzyl alcohol | 0.1 | — | 0.1 | — |
| Zinc citrate trihydrate | 0.5 | 0.5 | 0.5 | 0.5 |
| Zinc oxide | 1.0 | 1.0 | 1.0 | 1.0 |
| Sodium Fluoride-USP, EP | 0.32 | 0.32 | 0.32 | 0.32 |
| Glutamine | 0.3 | 0.3 | 0.3 | 0.3 |
| L-Arginine | — | — | 1.5 | 5.0 |
| Demineralized water | QS | QS | QS | QS |

Formulation Table N

| Ingredient | Composition W′ | Composition X′ | Composition Y′ | Composition Z′ |
|---|---|---|---|---|
| Humectants | 45.0-55.0 | 35.0-45.0 | 45.0-55.0 | 35.0-45.0 |
| Abrasives | 14.0-16.0 | 9.0-11.0 | 14.0-16.0 | 9.0-11.0 |
| Anionic surfactant | 1.0-3.0 | 1.0-3.0 | 1.0-3.0 | 1.0-3.0 |
| Non-ionic surfactant | 0.1-1.0 | — | 0.1-1.0 | — |
| Amphoteric surfactant | 1.0-2.0 | — | 1.0-2.0 | — |
| Flavoring/fragrance/coloring agent | 1.0-3.0 | 2.0-4.0 | 1.0-3.0 | 2.0-4.0 |
| Polymers | 0.1-2.0 | 3.0-8.0 | 0.1-2.0 | 3.0-8.0 |
| pH adjusting agents | 0.1-2.0 | 4.0-8.0 | 0.1-2.0 | 4.0-8.0 |
| Silica Thickener | 5.0 | 5.0-10.0 | 5.0 | 5.0-10.0 |
| Benzyl alcohol | 0.1 | — | 0.1 | — |
| Zinc citrate trihydrate | 0.5 | 0.5 | 0.5 | 0.5 |
| Zinc oxide | 1.0 | 1.0 | 1.0 | 1.0 |
| Sodium Fluoride-USP, EP | 0.32 | 0.32 | 0.32 | 0.32 |
| Glycine | 0.3 | 0.3 | 0.3 | 0.3 |
| L-Arginine | — | — | 1.5 | 5.0 |
| Demineralized water | QS | QS | QS | QS |

Formulation Table O

| Ingredient | Composition W″ | Composition X″ | Composition Y″ | Composition Z″ |
|---|---|---|---|---|
| Humectants | 45.0-55.0 | 35.0-45.0 | 45.0-55.0 | 35.0-45.0 |
| Abrasives | 14.0-16.0 | 9.0-11.0 | 14.0-16.0 | 9.0-11.0 |
| Anionic surfactant | 1.0-3.0 | 1.0-3.0 | 1.0-3.0 | 1.0-3.0 |
| Non-ionic surfactant | 0.1-1.0 | — | 0.1-1.0 | — |
| Amphoteric surfactant | 1.0-2.0 | — | 1.0-2.0 | — |
| Flavoring/fragrance/coloring agent | 1.0-3.0 | 2.0-4.0 | 1.0-3.0 | 2.0-4.0 |
| Polymers | 0.1-2.0 | 3.0-8.0 | 0.1-2.0 | 3.0-8.0 |
| pH adjusting agents | 0.1-2.0 | 4.0-8.0 | 0.1-2.0 | 4.0-8.0 |
| Silica Thickener | 5.0 | 5.0-10.0 | 5.0 | 5.0-10.0 |
| Benzyl alcohol | 0.1 | — | 0.1 | — |
| Zinc citrate trihydrate | 0.5 | 0.5 | 0.5 | 0.5 |
| Zinc oxide | 1.0 | 1.0 | 1.0 | 1.0 |
| Sodium Fluoride-USP, EP | 0.32 | 0.32 | 0.32 | 0.32 |
| Asparagine | 0.3 | 0.3 | 0.3 | 0.3 |
| L-Arginine | — | — | 1.5 | 5.0 |
| Demineralized water | QS | QS | QS | QS |

FORMULATION TABLE P

| Ingredient | P-1 | P-7 | P-3 | P-4 | P-5 | P-6 | P-7 | P-8 | P-9 |
|---|---|---|---|---|---|---|---|---|---|
| Humectants | 20.0- | 20.0- | 20.0- | 20.0- | 20.0- | 20.0- | 20.0- | 20.0- | 20.0- |
| | 50.0 | 50.0 | 50.0 | 50.0 | 50.0 | 50.0 | 50.0 | 50.0 | 50.0 |
| Abrasives | 5.0- | 5.0- | 5.0- | 5.0- | 5.0- | 5.0- | 5.0- | 5.0- | 5.0- |
| | 20.0 | 20.0 | 20.0 | 20.0 | 20.0 | 20.0 | 20.0 | 20.0 | 20.0 |
| Anionic surfactant | 1.0-3.0 | 1.0-3.0 | 1.0-3.0 | 1.0-3.0 | 1.0-3.0 | 1.0-3.0 | 1.0-3.0 | 1.0-3.0 | 1.0-3.0 |
| Non-ionic surfactant | 0.1-1.0 | 0.1-1.0 | 0.1-1.0 | 0.1-1.0 | 0.1-1.0 | 0.1-1.0 | 0.1-1.0 | 0.1-1.0 | 0.1-1.0 |
| Amphoteric surfactant | 0.1-2.0 | 0.1-2.0 | 0.1-2.0 | 0.1-2.0 | 0.1-2.0 | 0.1-2.0 | 0.1-2.0 | 0.1-2.0 | 0.1-2.0 |
| Flavoring/fragrance/ | 1.0-5.0 | 1.0-5.0 | 1.0-5.0 | 1.0-5.0 | 1.0-5.0 | 1.0-5.0 | 1.0-5.0 | 1.0-5.0 | 1.0-5.0 |
| coloring agent | | | | | | | | | |
| Polymers | 0.1-2.0 | 0.1-2.0 | 0.1-2.0 | 0.1-2.0 | 0.1-2.0 | 0.1-2.0 | 0.1-2.0 | 0.1-2.0 | 0.1-2.0 |
| pH adjusting agents | 0.1-2.0 | 0.1-2.0 | 0.1-2.0 | 0.1-2.0 | 0.1-2.0 | 0.1-2.0 | 0.1-2.0 | 0.1-2.0 | 0.1-2.0 |
| Thickener | 6.0 | 6.5 | 7.0 | 6.0 | 6.5 | 7.0 | 6.0 | 6.5 | 7.0 |
| Dental type silica | — | — | 15.0 | — | — | 15.0 | — | — | 15.0 |
| High cleaning silica | — | 15.0 | — | — | 15.0 | — | — | 15.0 | — |
| Synthetic Abrasives | 10.0 | — | — | 10.0 | — | — | 10.0 | — | — |
| Synthetic Amorphous | 5.0 | 5.0 | 5.0 | 5.0 | 5.0 | 5.0 | 5.0 | 5.0 | 5.0 |
| Silica | | | | | | | | | |
| Benzyl alcohol | 0.4 | 0.4 | 0.4 | 0.4 | 0.4 | 0.4 | 0.4 | 0.4 | 0.4 |
| Zinc citrate trihydrate | 0.5 | 0.5 | 0.5 | 0.5 | 0.5 | 0.5 | 0.5 | 0.5 | 0.5 |
| Zinc oxide | 1.0 | 1.0 | 1.0 | 1.0 | 1.0 | 1.0 | 1.0 | 1.0 | 1.0 |
| Sodium Fluoride- | 0.32 | 0.32 | 0.32 | 0.32 | 0.32 | 0.32 | 0.32 | 0.32 | 0.32 |
| USP, EP | | | | | | | | | |
| Glutamine | 0.5 | 0.5 | 0.5 | — | — | — | — | — | — |
| Glycine | — | — | — | 0.5 | 0.5 | 0.5 | — | — | — |
| Asparagine | — | — | — | — | — | — | 0.5 | 0.5 | 0.5 |
| Demineralized water | QS | QS | QS | QS | QS | QS | QS | QS | QS |

### Example 14

The gingival epithelium acts as a mechanical barrier between the external environment containing oral pathogens and their toxins and the underlying connective tissue. It thus plays a key functional role in maintaining oral health. An in vitro gingival keratinocyte model to investigate the effects of formulations (aqueous solution and dentifrice) comprising zinc oxide, zinc citrate and arginine formulations on TNF-α-induced barrier dysfunction as well as on cell proliferation and migration.

Gingival keratinocytes were seeded onto the membrane of a double-chamber system in the absence and presence of recombinant human TNF-α and the formulations under investigation. The barrier function was assessed by determination of transepithelial electrical resistance (TER) and paracellular transport of FITC-dextran. The distribution of zonula occludens-1 (ZO-1) and occludin, which were chosen as markers of the tight junction, was visualized by immunofluorescence microscopy. The effects of the zinc oxide, zinc citrate and arginine formulations on keratinocyte cell proliferation were determined using a fluorescent cell tracker dye, while a migration assay kit was used to investigate their effects on cell migration.

Under conditions where TNF-α induces loss of keratinocyte barrier integrity, two different formulations which each comprise zinc oxide, zinc citrate and arginine (aqueous solution and dentifrice) protected the keratinocyte tight junction against the damages since they prevented the TNF-α-induced drop in TER values and increase in FITC-dextran paracellular flux in the Transwell™ based in vitro model of keratinocyte barrier. The treatment of keratinocytes with the formulations markedly mitigated the altered distribution of ZO-1 and occludin compared to cells exposed only to TNF-α. Both of the formulations (aqueous solution and dentifrice) increased the cell proliferation of gingival keratinocytes and alleviated the negative impact caused by TNF-α. Lastly, the formulations increased the migration capacity of gingival keratinocytes. Zinc oxide, zinc citrate and arginine formulations (aqueous solution and dentifrice) protected the barrier integrity of gingival keratinocytes from TNF-α-induced damage and promoted their proliferation and migration.

### Materials and Methods

### Formulations

Zinc oxide and zinc citrate trihydrate were obtained from U.S. Zinc (Houston, Tex., USA) and Jost Chemical (St. Louis, Mo., USA), respectively. L-arginine was purchased from Ajinomoto (Tokyo, Japan). A mixture containing 0.96% zinc (zinc oxide, zinc citrate) and 1.5% arginine, referred to as the aqueous solution, was freshly prepared in sterile distilled water. Unless indicated otherwise, the aqueous solution was used at dilutions of 1/500, 1/1000, and 1/2000 (v/v). A dentifrice containing 0.96% zinc (zinc oxide, zinc citrate), 1.5% arginine, and 1450 ppm fluoride as sodium fluoride in a silica base, was also investigated. A zinc- and arginine-free fluoride dentifrice was included in the study as a control. Unless indicated otherwise, the dentifrice and control fluoride dentifrice were used at dilutions of 1/500, 1/1000, and 1/2000 (w/v). At the dilutions used, the amounts of zinc and arginine in the aqueous solution and the dentifrice were comparable.

### Human Gingival Keratinocyte Culture

To investigate the protective effect of the aqueous solution and dentifrice on TNF-α-induced keratinocyte barrier dysfunction, the B11 immortalized human gingival keratinocyte cell line was used. Cells were cultivated in keratinocyte serum-free medium (K-SFM; Life Technologies Inc., Burlington, ON, Canada) containing growth factors (50 μg/ml of bovine pituitary extract and 5 ng/ml of human epidermal growth factor) and 100 μg/ml of penicillin G-streptomycin. Cultures were incubated in a 5% CO₂ atmosphere at 37° C.

Determination of transepithelial electrical resistance

Keratinocyte barrier integrity was determined by monitoring transepithelial electrical resistance (TER). Briefly, keratinocytes (3×10⁵ cells per insert; 0.1 ml) were seeded in the apical compartment of Costar™ Transwell™ clear polyester membrane inserts (6.5 mm in diameter, 0.4 μm pore size; Corning Co., Cambridge, Mass., USA), while the basolateral compartment was filled with 0.6 ml of culture medium. After 72-h of incubation, the conditioned culture medium was replaced with fresh antibiotic-free K-SFM, and the cells were further incubated for 16 h prior treating the cells. Recombinant human TNF-α (100 ng/ml; AnaSpec, Fremont, USA) and the formulations (final dilutions of 1/500, 1/1000, and 1/2000 (v/v for the aqueous solution; w/v for the dentifrice) under investigation were added to the apical compartment. The TER values were monitored using an ohm/voltmeter (EVOM²; World Precision Instruments, Sarasota, Fla., USA) after 0, 6, 24, and 48 h of incubation. The TER values in Ohms (Ω)/cm² were determined by multiplying the resistance values by the surface area of the membrane filter. Results are expressed as the percentage of the basal control value measured at time 0-h (100% value).

### Determination of Fluorescein Isothiocyanate-Conjugated Dextran Transport

Gingival keratinocytes were cultured in the apical chamber of Transwell™ inserts as described above. Immediately after adding fluorescein isothiocyanate-conjugated dextran of 4.4 kDa (FD-4; 1 mg/ml in culture medium) to the apical compartment, the formulations under investigation were added in presence of recombinant TNF-α (100 ng/ml). Fluorescence in the basolateral compartment was recorded after 0, 2, 6, 24, and 48 h of incubation using a Synergy 2 microplate reader (BioTek Instruments; Winooski, Vt., USA).

### Distribution of Occludin and Zonula Occludens-1 by Immunofluorescence Analysis

For the immunofluorescence analysis, keratinocytes were treated for 24 h as described above and immunostained for two tight junction proteins, occludin and zonula occludens-1 (ZO-1) according to the protocol described by Ben Lagha, A., & Grenier, D. (2019). Tea polyphenols protect gingival keratinocytes against TNF-α-induced tight junction barrier dysfunction and attenuate the inflammatory response of monocytes/macrophages. Cytokine, 115, 64-75. The distribution of occludin and ZO-1 in the keratinocyte layer was visualized using an inverted Olympus FSX100 fluorescent microscope (Olympus, Tokyo, Japan).

### Determination of Keratinocyte Proliferation

Gingival keratinocytes were seeded into wells (20,000 cells per well) of a 96-well microplate (0.1 ml/well) and incubated overnight at 37° C. in a 5% CO₂ atmosphere to allow cell adhesion. The culture medium was then removed, and the cells were treated or not with TNF-α (100 ng/ml) with or without the formulations under investigation for 72 h at 37° C. in a 5% CO₂ atmosphere. Cell proliferation was measured using the fluorescent CellTracker™ Green CMFDA (5-chloromethylfluorescein diacetate) Dye according to the manufacturer's instructions (Thermo Fisher Scientific). Untreated control cells were assigned a value of 100%. Briefly, after 24 h of stimulation, the supernatant was removed and the cells were washed with PBS. To examine the effect of the tested formulations (1/128,000, 1/64,000 and 1/32,000 dilutions) on cell proliferation, the CellTracker reagent (10 mM) was added directly to each well (0.1 ml/well). After 30 min of staining, the CellTracker working solution was removed, the cells were washed with PBS and the fluorescence was monitored using a Synergy 2 microplate reader (485 nm/528 nm; excitation/emission wavelengths). Fluorescent keratinocytes were also visualized using an inverted Olympus FS X100 fluorescent microscope.

### Determination of Keratinocyte Migration

The ability of the formulations (aqueous solution and dentifrice) to promote keratinocyte migration was assessed using the Oris™ Pro Cell Migration Assay kit (Platypus Technologies, Madison, Wis., USA) according to the manufacturer's protocol. Briefly, 1×10⁶ gingival keratinocytes (200 μl) were seeded into wells of a 96-well microplate. The type I collagen-coated wells were equipped with in-place stoppers to create a migration area on the bottom of each well. The stoppers were removed after an overnight incubation, the medium was aspirated, and fresh medium containing the formulations (1/8000 and 1/4000 dilutions) under investigation was added. After an incubation of 24 h, the cells were washed twice with sterile PBS containing both Mg²⁺ and Ca′ and stained with CellTracker™ Green CMFDA Dye according to the manufacturer's instructions (Thermo Fisher Scientific). Cell migration was quantified using a microplate reader (485 nm/528 nm; excitation/emission wavelengths) with a black bottom mask provided by the manufacturer.

Statistical Analysis

Unless indicated otherwise, all experiments were performed in triplicate in three independent experiments. The data are expressed as means±standard deviations (SD). Statistical analyses were performed using a one-way analysis of variance with a post hoc Bonferroni multiple comparison test (GraphPad Software Inc., La Jolla, Calif., USA). All results were considered statistically significant at p<0.01.

Results

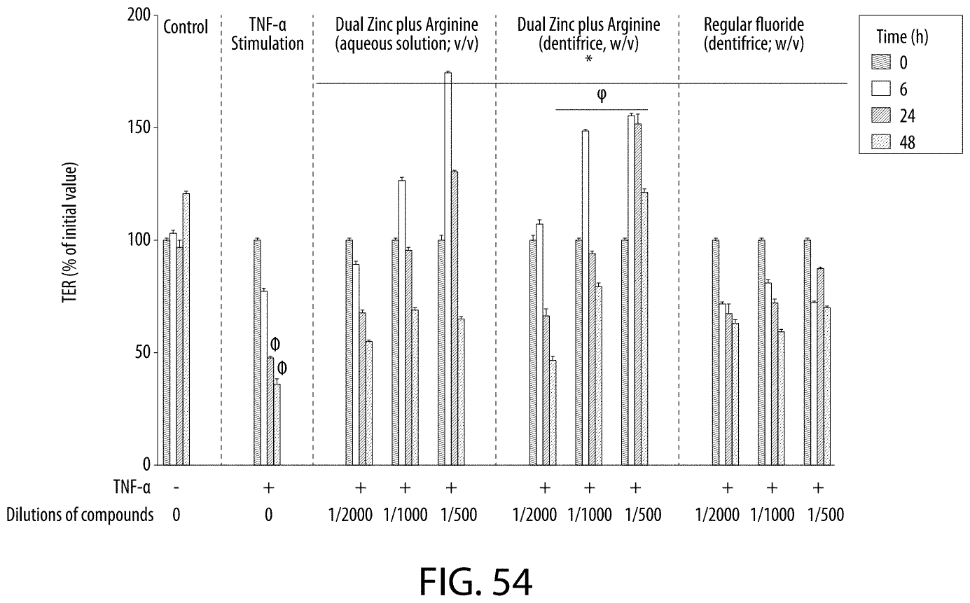

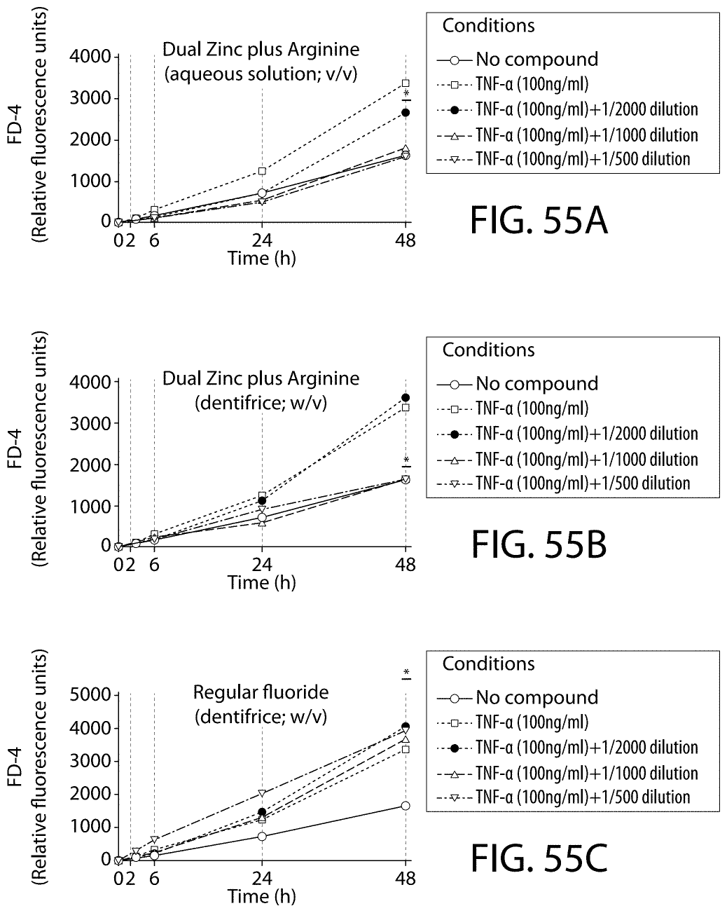

Using a Transwell™ based in vitro model with the human keratinocyte cell line B11, whether the formulations (aqueous solution and dentifrice) could prevent the negative impact of TNF-α on the keratinocyte barrier integrity was investigated first. As reported in FIG. 54, a treatment of the gingival keratinocytes with TNF-α at 100 ng/ml significantly and time-dependently decreased the TER values. More specifically, TER was decreased by 25.8, 49.3, and 85.1% after 6, 24, and 48 h of exposure of cells to TNF-α, respectively. Correspondingly, a significant increase in paracellular transport of FD-4 across the gingival keratinocyte layer, from the apical to the basolateral chamber was observed; following a 48-h exposure, the transport of FD-4 was increased by 50.8% (FIGS. 55A, 55B and 55C). Despite these deleterious effects of TNF-α on the keratinocyte barrier integrity, no significant loss of cell viability was observed using an MTT (3-[4,5-diethylthiazol-2-yl]-2,5diphenyltetrazolim bromide) colorimetric assay that determines cell metabolic activity (data not shown).

Both formulations (aqueous solution and dentifrice) dose-dependently prevented the TNF-α-induced decrease of TER values. Moreover, at dilutions 1/500 and 1/1000, the formulations significantly increased the TER above the control values associated with keratinocytes not treated with TNF-α. Following a 24-h treatment of gingival keratinocytes with TNF-α, the presence of the aqueous solution and dentifrice at dilution 1/500 increased the TER values by 83.0 and 104.3% when compared to cells treated with TNF-α alone. To a much lesser extent, the control dentifrice also prevented the TNF-α-induced decrease in TER.

The ability of the formulations (aqueous solution and dentifrice) to attenuate the deleterious effects of TNF-α on the keratinocyte barrier function was confirmed by measuring the paracellular transport of FITC-dextran. Adding the formulations at dilutions 1/500 and 1/1000 prevented the TNF-α-mediated increase in FITC-dextran paracellular transport. More specifically, a 48-h treatment with a 1/500 dilution of the Dual Zinc plus Arginine aqueous solution and dentifrice caused 2.1- and 2.0-fold decrease in FITC-dextran transport associated to the presence of TNF-α, respectively. Such effect was not observed with the control fluoride dentifrice.

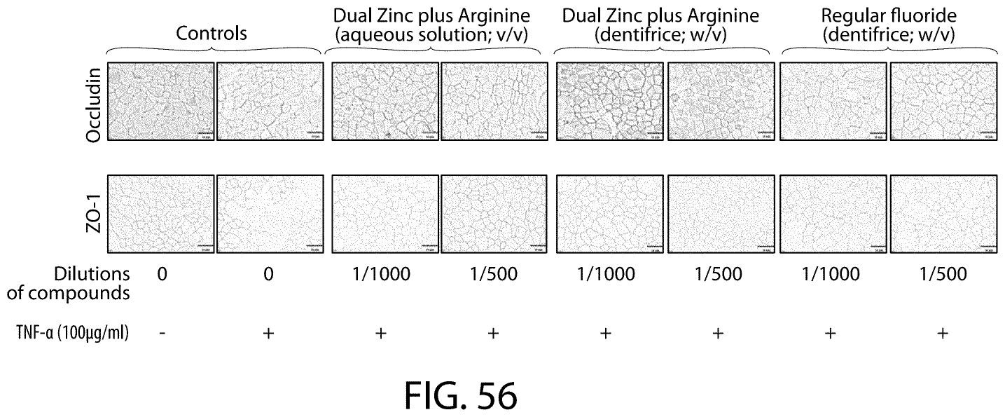

Immunofluorescence staining of occludin and ZO-1 was performed to assess the effect of the formulations (aqueous solution and dentifrice) on the distribution of these two tight junction proteins in TNF-α-treated keratinocytes. As shown in FIG. 56, in control cells, occludin and ZO-1 were located at the cell membrane and formed continuous structures. The TNF-α treatment induced a disturbed and irregular cellular distribution of the two proteins in keratinocytes. However, the presence of the formulations markedly mitigated the altered distribution of occludin and ZO-1 compared to cells treated with TNF-α only. These effects were much less evident with the control fluoride dentifrice.

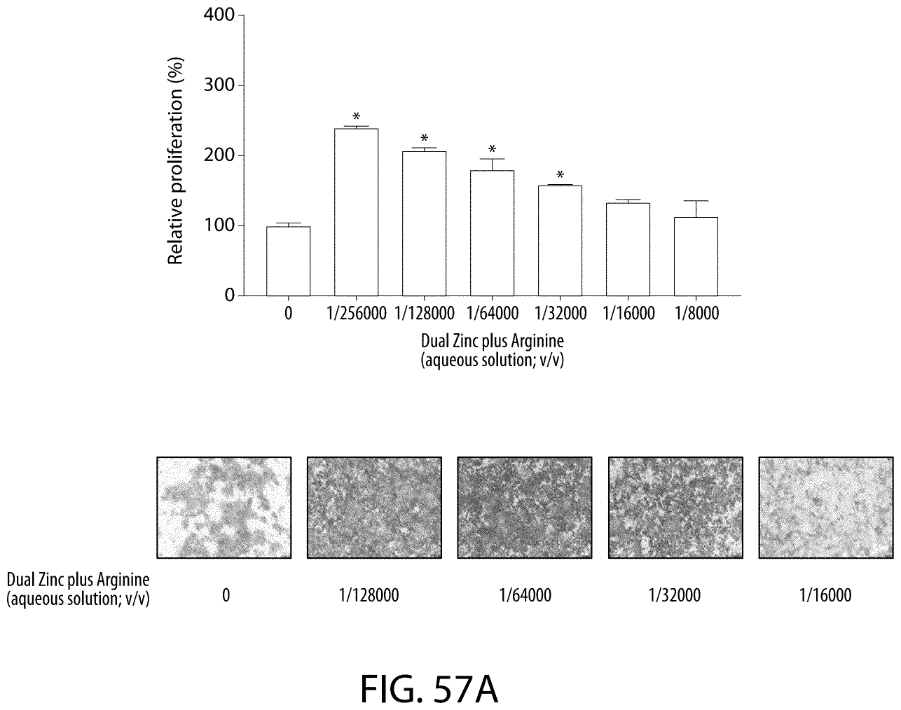

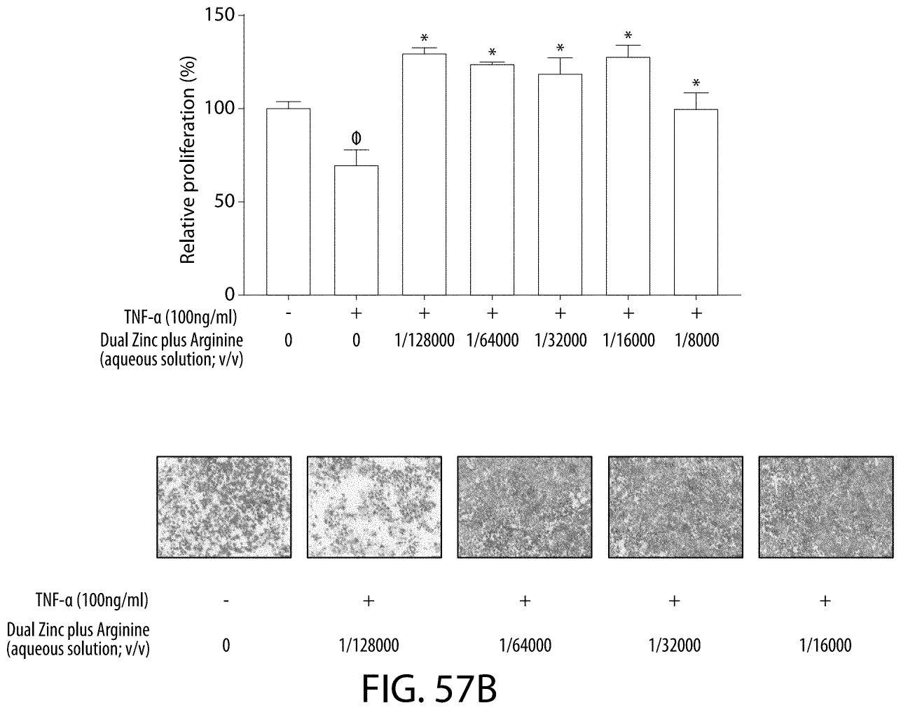

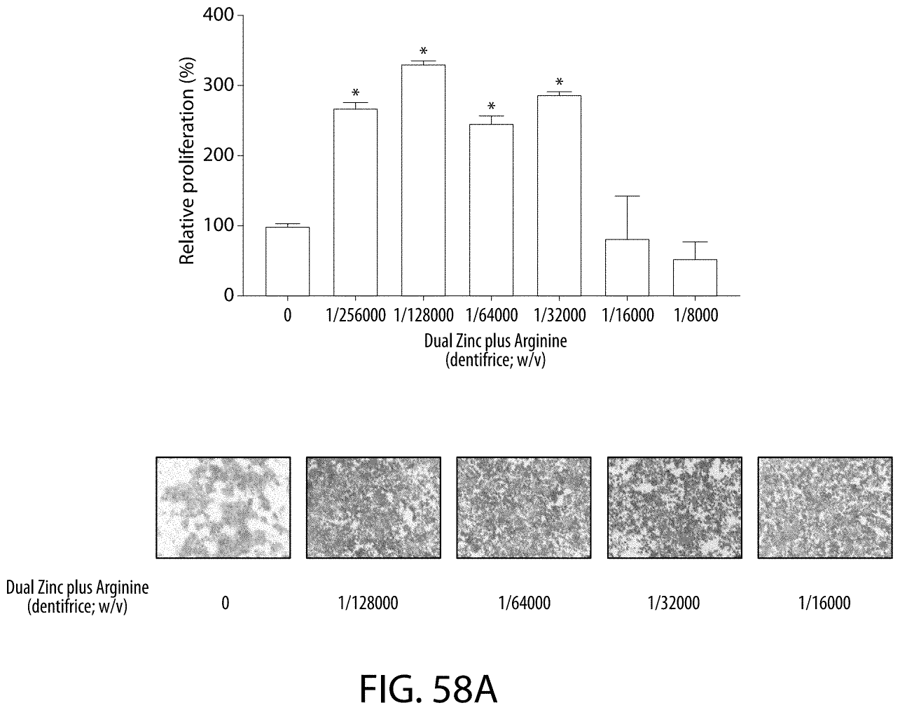

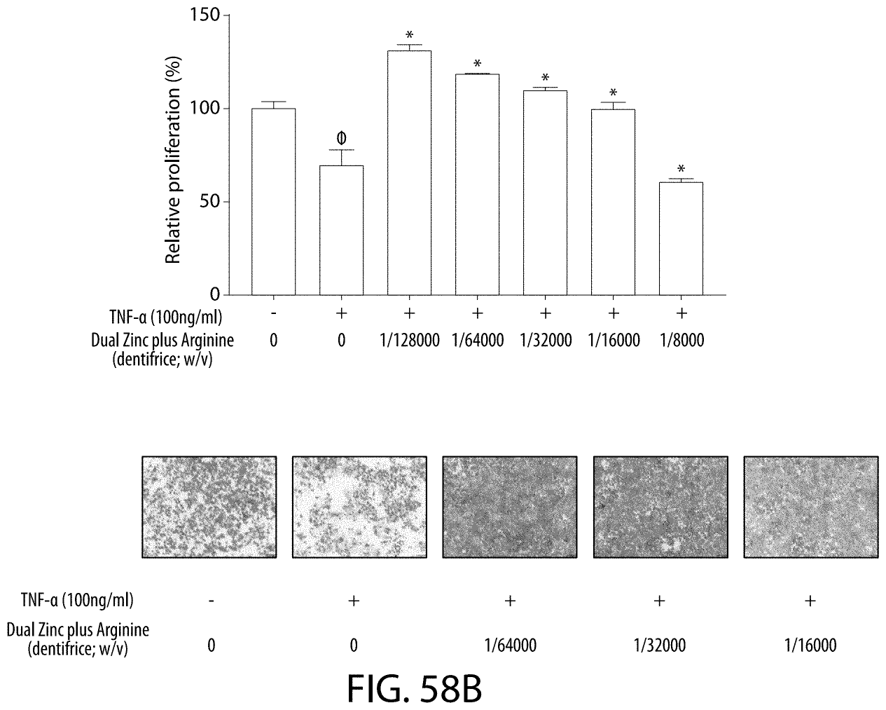

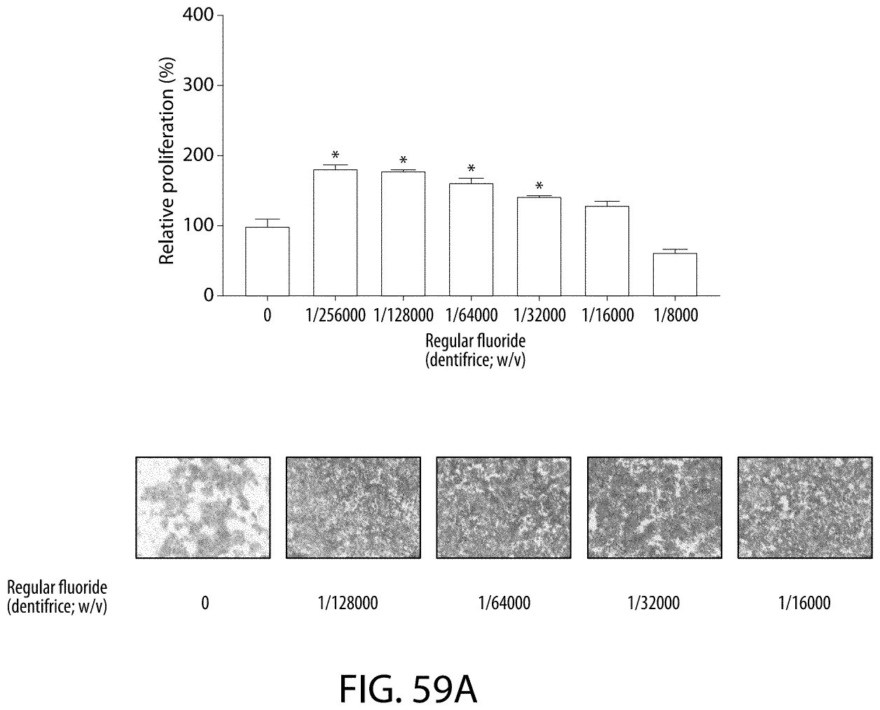

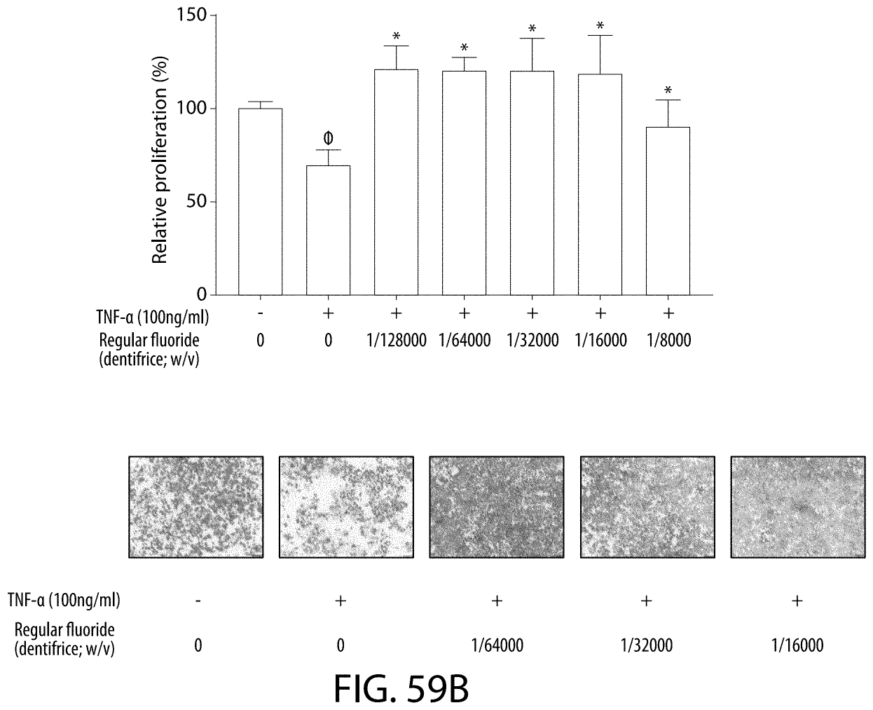

The effects of the formulations (aqueous solution and dentifrice) on the proliferation of gingival keratinocytes using a fluorescent dye were investigated next. The proliferative effects were more pronounced when higher dilutions of the formulations were used. At a 1/256,000 dilution, the aqueous solution and dentifrice increased the cell proliferation by 140.1 and 168.1%, respectively (FIGS. 57A and 58A). To a lesser extent, the control fluoride dentifrice used at the same dilution also promoted the proliferation of keratinocytes (81.0% increase) (FIG. 59A). Treating the gingival keratinocytes with TNF-α reduced the cell proliferation by 30.2% (FIG. 57B). Both formulations as well as the control fluoride dentifrice prevented this TNF-α-mediated reduction in cell proliferation (FIGS. 57B, 58B and 59B).

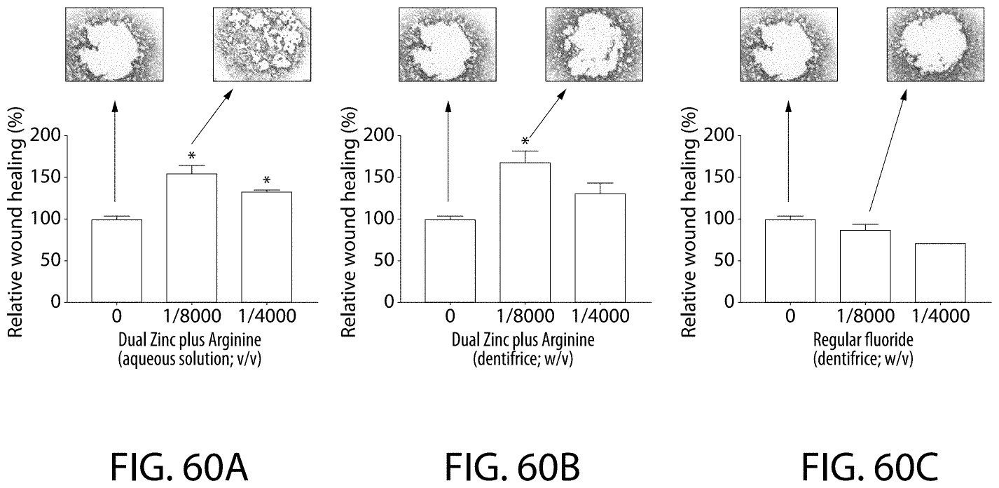

Lastly, the effects of the formulations (aqueous solution and dentifrice) on the gingival keratinocyte migration were investigated. FIGS. 60A, 60B and 60C shows that both formulations increased cell migration. More specifically, at a 1/8000 dilution, the aqueous solution (FIG. 60A), and dentifrice (FIG. 60B) increased the cell migration by 54.6 and 68.0%, respectively. Such effect was not observed with the control fluoride dentifrice (FIG. 60C).

### DISCUSSION

Periodontal disease is a biofilm-induced inflammatory condition of the tooth-supporting tissue that involves complex interactions between periodonto-pathogenic bacteria and mucosal and immune host cells. The inflammatory cytokine network is responsible for the activation of several cell signaling pathways leading to periodontal soft tissue and bone destruction. More specifically, the pro-inflammatory cytokine TNF-α is thought to be a central component involved in the pathogenesis of periodontal disease. The extremely pleiotropic nature of TNF-α action could be ascribed to the presence of TNF receptors on a large array of cell types leading to an activation of multiple signal transduction pathways, kinases and transcription factors. In this regard, evidence has been obtained that TNF-α antagonists may potentially reduce tissue destruction associated with periodontal disease.

The oral epithelial tight junction barrier prevents the translocation of bacteria and their toxins in the underlying periodontal connective tissues. Using in vitro models, TNF-α has been shown to induce dysfunction of the gingival keratinocyte barrier through modulation of the distribution and expression of tight junction proteins. Given that the regulation of the keratinocyte barrier may represent a strategy for preventing the onset of periodontal disease, periodontal inflammation, and translocation of bacteria to non-oral sites, the effects of a zinc oxide, zinc citrate and arginine aqueous solution and a zinc oxide, zinc citrate and arginine dentifrice on the TNF-α-mediated disruption of the barrier function of an in vitro gingival keratinocyte model were investigated. The impact of these formulations on keratinocyte proliferation and migration was also determined.

The Transwell™ based in vitro model with oral keratinocytes was used to show that TNF-α caused a reduction of TER that coincided with the increase of FITC-dextran paracellular transport, two complementary indicators of barrier dysfunction. Although the molecular mechanisms associated with this TNF-α-induced permeability of the keratinocyte barrier have not been investigated, several studies related to this aspect with regard to the human intestinal epithelial barrier have been published. These studies brought evidence that TNF-α affects intestinal permeability by modulating different signaling pathways, particularly NF-κB, affecting the functionality and structure of selected tight junction proteins.

The zinc oxide, zinc citrate and arginine formulations protected the keratinocyte barrier against this TNF-α-mediated disruption. Indeed, the formulations prevented the TNF-α-induced drop in TER values and increase in FD-4 paracellular flux in the Transwell™ based in vitro keratinocyte barrier model.

An immunofluorescence analysis of tight junction proteins showed that TNF-α alters the distribution of occludin and ZO-1 in gingival keratinocytes. In contrast, the treatment with the zinc oxide, zinc citrate and arginine aqueous solution and the zinc oxide, zinc citrate and arginine dentifrice largely prevented this TNF-α induced change in the morphological distribution of these two proteins used as markers of tight junction. On the one hand, ZO-1 plays a key role in tight junction functions by connecting transmembrane proteins such as occludin, to other cytoplasmic tight junction proteins and to the cytoskeleton. On the other hand, occludin is a membrane protein with two extracellular loops that interconnect with ZO-1. These two proteins are essential for cell-to-cell interactions and in maintaining the epithelial barrier function.

The ability of the formulations to promote gingival the keratinocyte barrier function may be zinc-related. Using an in vitro oral mucosa model, Rybakovsky 2017 (Rybakovsky E, et al. 2017. Improvement of human-oral-epithelial-barrier function and of tight junctions by micronutrients. J. Agric Food Chem 65:10950-10958) investigated the effects of selected micronutrients on transepithelial electrical resistance and reported the ability of zinc to improve epithelial barrier function. Moreover, using in vivo (mouse) and in vitro models, Sarkar 2019 (Sarkar P, et al. 2019. Zinc ameliorates intestinal dysfunctions in shigellosis by reinstating claudin-2 and -4 on the membranes. Am J Physiol Gastrointest Liver Physiol 316:G229-G246.) demonstrated that the presence of zinc allows protection of the intestinal epithelial barrier from damage mediated by the pathogen Shigella flexneri. This protective effect was associated with the ability of zinc to inhibit the S. flexneri-phosphorylation of extracellular signal-regulated kinase (ERK)1/2, which causes disengagement of claudin-2 and -4 and consequently barrier dysfunction. L-arginine, a component of the formulations tested, has been reported to improve the intestinal mucosal barrier functions through activation of AMP kinase signaling that results in an enhanced expression of tight junction proteins, including ZO-1 and claudin-1.

The ability of compounds to protect the gingival keratinocyte barrier against the damages induced by TNF-α has been previously reported. Ben Lagha A & Grenier D. 2019 supra showed that tea polyphenols, including epigallocatechin-3-gallate and theaflavins, prevented epithelial barrier disruption by reducing TNF-α-induced alterations on TER and FITC-dextran paracellular transport. Similarly, irsogladine maleate, an anti-gastric agent known to enhance gap junctional intercellular interactions, also reversed the TNF-α-induced disruption of the gingival epithelial barrier by regulating E-cadherin and claudin-1 expression.

Among the many steps of periodontal repair, the process of re-epithelialization is critical for the recovery of intact gingival barrier function. The formulations (aqueous solution and dentifrice) tested significantly increased the proliferation and migration of gingival keratinocytes. A chronic non-healing wound that would otherwise offer an opportunity for pathogens to adhere, colonize, invade, and infect surrounding healthy tissue may be prevented.

The ability of the formulations (aqueous solution and dentifrice) to attenuate the virulence properties of P. gingivalis and protect the keratinocyte barrier function against the damage mediated by bacterial proteases has been shown. The formulations can protect the barrier integrity of gingival keratinocytes from TNF-α-induced damage in addition to promoting their proliferation and migration. All together, these properties are consistent with the ability of the formulations comprising zinc oxide, zinc citrate and arginine (aqueous solution and dentifrice) to offer benefits for preventing periodontal disease and to attenuate the virulence properties of P. gingivalis and protect the keratinocyte barrier integrity.

1. A method of improving tissue integrity of tissue that functions as a barrier comprising contacting the tissue with an effective amount of a composition comprising one or more sources of zinc ions, and optionally further comprising one or more amino acids selected from the group consisting of: arginine, alanine, asparagine, cysteine, glutamine, glycine, isoleucine, leucine, methionine, phenylalanine, proline, serine, threonine, tryptophan, tyrosine, valine, and amino acids which have an isoelectric point in range of pH 5.0 to 7.0.

2. The method of claim 1 wherein the one or more sources of zinc ions is selected from the group consisting of: zinc chloride, zinc acetate, zinc gluconate, zinc sulphate, zinc fluoride, zinc citrate, zinc lactate, zinc oxide, zinc monoglycerolate, zinc tartrate, zinc pyrophosphate, zinc phosphate, zinc maleate, zinc malate, zinc carbonate, zinc ascorbate, zinc lysine hydrochloride and zinc chloride hydroxide monohydrate (TBZC).

3. The method of claim 1 comprising contacting the tissue with an effective amount of a composition comprising one or more sources of zinc ions selected from the group consisting of zinc oxide and zinc citrate, and optionally further comprising arginine.

4. A method of repairing damage to tissue that functions as a barrier in an individual that has damage to tissue that functions as a barrier comprising contacting the tissue with an effective amount of a composition comprising one or more sources of zinc ions, and optionally further comprising one or more amino acids selected from the group consisting of: arginine, alanine, asparagine, cysteine, glutamine, glycine, isoleucine, leucine, methionine, phenylalanine, proline, serine, threonine, tryptophan, tyrosine, valine, and amino acids which have an isoelectric point in range of pH 5.0 to 7.0.

5. The method of claim 4 wherein the one or more sources of zinc ions is selected from the group consisting of: zinc chloride, zinc acetate, zinc gluconate, zinc sulphate, zinc fluoride, zinc citrate, zinc lactate, zinc oxide, zinc monoglycerolate, zinc tartrate, zinc pyrophosphate, zinc phosphate, zinc maleate, zinc malate, zinc carbonate, zinc ascorbate, zinc lysine hydrochloride and zinc chloride hydroxide monohydrate (TBZC).

6. The method of claim 4 comprising contacting the tissue with an effective amount of a composition comprising one or more sources of zinc ions selected from the group consisting of zinc oxide and zinc citrate, and optionally further comprising arginine.

7. The method of claim 4 wherein the damage has been caused by the presence of proinflammatory cytokines.

8. The method of claim 4 wherein the damage has been induced by TNF-α.

9. The method of claim 4 wherein the damage has been caused by pathogenic bacteria.

10. The method of claim 4 wherein the damage has been caused by collagenase activity.

11. The method of claim 4 wherein the tissue damage has been caused by hemolytic activity.

12. The method of claim 4 wherein the damage has been caused by induction of proteases in host cells.

13. The method of claim 4 wherein the tissue is contacted with an amount of the composition sufficient to promote keratinocyte proliferation and keratinocyte migration.

14. A method of protecting and maintaining tissue integrity of tissue that functions as a barrier comprising contacting the tissue with an effective amount of a composition comprising one or more sources of zinc ions, and optionally further comprising one or more amino acids selected from the group consisting of: arginine, alanine, asparagine, cysteine, glutamine, glycine, isoleucine, leucine, methionine, phenylalanine, proline, serine, threonine, tryptophan, tyrosine, valine, and amino acids which have an isoelectric point in range of pH 5.0 to 7.0.

15. The method of protecting and maintaining tissue integrity of tissue that functions as a barrier according to claim 14 wherein the one or more sources of zinc ions is selected from the group consisting of: zinc chloride, zinc acetate, zinc gluconate, zinc sulphate, zinc fluoride, zinc citrate, zinc lactate, zinc oxide, zinc monoglycerolate, zinc tartrate, zinc pyrophosphate, zinc phosphate, zinc maleate, zinc malate, zinc carbonate, zinc ascorbate, zinc lysine hydrochloride and zinc chloride hydroxide monohydrate (TBZC)

16. The method of protecting and maintaining tissue integrity of tissue that functions as a barrier according to claim 14 comprising contacting the tissue with an effective amount of a composition comprising one or more sources of zinc ions selected from the group consisting of zinc oxide and zinc citrate, and optionally further comprising arginine.

17. A method of improving oral immunity provided by a tissue that functions as a barrier comprising contacting the tissue with an effective amount of a composition comprising one or more sources of zinc ions, and optionally further comprising one or more amino acids selected from the group consisting of: arginine, alanine, asparagine, cysteine, glutamine, glycine, isoleucine, leucine, methionine, phenylalanine, proline, serine, threonine, tryptophan, tyrosine, valine, and amino acids which have an isoelectric point in range of pH 5.0 to 7.0.

18. The method of improving oral immunity provided by a tissue that functions as a barrier according to claim 17 wherein the one or more sources of zinc ions is selected from the group consisting of: zinc chloride, zinc acetate, zinc gluconate, zinc sulphate, zinc fluoride, zinc citrate, zinc lactate, zinc oxide, zinc monoglycerolate, zinc tartrate, zinc pyrophosphate, zinc phosphate, zinc maleate, zinc malate, zinc carbonate, zinc ascorbate, zinc lysine hydrochloride and zinc chloride hydroxide monohydrate (TBZC)

19. The method of improving oral immunity provided by a tissue that functions as a barrier according to claim 17 comprising contacting the tissue with an effective amount of a composition comprising one or more sources of zinc ions selected from the group consisting of zinc oxide and zinc citrate, and optionally further comprising arginine.

20. The method of claim 14 wherein the tissue is protected from tissue damage caused by pathogenic bacteria.

21. The method claim 14 wherein the tissue is protected from proinflammatory cytokine induced tissue damage.

22. The method of claim 14 wherein the tissue is protected from damage caused by collagenase activity.

23. The method of claim 14 wherein the tissue is protected from damage caused by hemolytic activity.

24. The method of claim 14 wherein the tissue is protected from damage caused by induction of proteases in host cells.

25. The method of claim 4 wherein the tissue is oral tissue.

26. The method of claim 25 wherein the tissue is oral epithelial barrier tissue.

27. The method of claim 25 wherein the tissue is gingival epithelial barrier tissue.

28. The method of claim 25 wherein the barrier is a keratinocyte tight junction barrier of oral epithelium.

29. The method of claim 4 wherein the method comprises contacted an individual's oral cavity with a composition comprising zinc oxide, or zinc citrate, or zinc oxide and zinc citrate and optionally arginine.

30. The method of claim 4 wherein the composition is an oral care composition.

31. The method of claim 4 wherein the composition is a toothpaste.

32. The method of claim 4 wherein:
the zinc oxide is present in an amount of from 0.75 to 1.25 wt %, or
the zinc citrate is present in an amount of from 0.25 to 1.0 wt %, or
the zinc oxide is present in an amount of from 0.75 to 1.25 wt % and the zinc citrate is present in an amount of from 0.25 to 1.0 wt %.

33. The method of claim 4 wherein the composition comprises arginine present in an amount of from 0.1% to 15%, based on the total weight of the composition, the weight of the amino acid being calculated as free form.

34. The method of claim 33 wherein the arginine is L-arginine.

35. The method of claim 33 wherein the arginine is in free form.

36. The method of claim 33 wherein the arginine is in salt form.

37. The method of claim 4, wherein composition comprises zinc oxide and zinc citrate and the zinc oxide is present in an amount of from 0.75 to 1.25 wt % and the zinc citrate is present in an amount of from 0.25 to 1.0 wt % and the ratio of the amount of zinc oxide (by wt %) to zinc citrate (by wt %) is 2:1, 2.5:1, 3:1, 3.5:1 or 4:1, based on the total weight of the composition.

38. The method of claim 37 wherein the ratio of the amount of zinc oxide (by wt %) to zinc citrate (by wt %) is 2:1, based on the total weight of the composition.

39. The method of claim 4 further comprising fluoride.

40. The method of claim 4 further comprising stannous fluoride.

41. The method of claim 4 further comprising the step of identifying the individual as having periodontal disease. gingivitis and in some embodiments, periodontitis.

42. The method of claim 4 further comprising the step of identifying the individual as having cardiovascular disease, respiratory diseases, type 2 diabetes periodontal disease, preterm birth/low birth weight, or colorectal disease.

43. The method of claim 4 further comprising the steps of identifying the individual as having Gram-negative anaerobic bacteria initiating disease in their oral cavity.

44. The method of claim 4 further comprising the steps of identifying the individual as experiencing chronic inflammation in their oral cavity.

45. The method of claim 4 further comprising the steps of identifying the individual as having damage to tissue that functions as a barrier.
